(19) United States
(12) Patent Application Publication
(10) Pub. No.: US 2018/0230127 A1
(43) Pub. Date: Aug. 16, 2018

(54) Bicyclic-Fused Heteroaryl Or Aryl Compounds

(71) Applicant: PFIZER INC., NEW YORK, NY (US)

(72) Inventors: DAVID RANDOLPH ANDERSON, Salem, CT (US); Kevin Joseph CURRAN, Somerset, NJ (US); Lori Krim GAVRIN, Villanova, PA (US); Joel Adam GOLDBERG, Cleveland Heights, OH (US); Arthur LEE, Redwood City, CA (US); Michael Dennis LOWE, Pleasantville, NY (US); Akshay Patny, Waltham, MA (US); Betsy Susan PIERCE, East Lyme, CT (US); Eddine SAIAH, Brookline, MA (US); John David TRZUPEK, Medford, MA (US)

(73) Assignee: PFIZER INC., NEW YORK, NY (US)

(21) Appl. No.: 15/751,310

(22) PCT Filed: Jul. 29, 2016
(86) PCT No.: PCT/IB2016/054571

§ 371 Date: Feb. 8, 2018

Related U.S. Application Data
(60) Provisional application No. 62/204,521, filed on Aug. 13, 2015.

(51) Int. Cl. C07D 401/12; C07D 211/94

(52) U.S. Cl. CPC C07D 401/12; A61K 31/437; A61K 2300/00; C07D 211/94

(57) ABSTRACT

Compounds, tautomers and pharmaceutically acceptable salts of the compounds of Formula (Ia) are disclosed which are inhibitors of Interleukin-1 receptor associated kinase (IRAK4). Methods of treatment, methods of synthesis, and intermediates are also disclosed as defined in the specification.

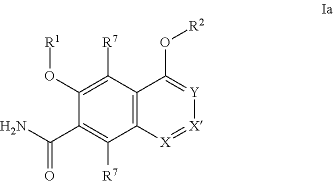

### FIELD OF THE INVENTION

This invention pertains to compounds useful for the treatment of autoimmune and inflammatory diseases associated with Interleukin-1 Receptor Associated Kinase (IRAK) and more particularly compounds that modulate the function of IRAK4.

### BACKGROUND OF THE INVENTION

Protein kinases are families of enzymes that catalyze the phosphorylation of specific residues in proteins, broadly classified in tyrosine and serine/threonine kinases. Inappropriate activity arising from dysregulation of certain kinases by a variety of mechanisms is believed to underlie the causes of many diseases, including but not limited to, cancer, cardiovascular diseases, allergies, asthma, respiratory diseases, autoimmune diseases, inflammatory diseases, bone diseases, metabolic disorders, and neurological and neurodegenerative diseases. As such, potent and selective inhibitors of kinases are sought as potential treatments for a variety of human diseases.

There is considerable interest in targeting the innate immune system in the treatment of autoimmune diseases and sterile inflammation. Receptors of the innate immune system provide the first line of defense against bacterial and viral insults. These receptors recognize bacterial and viral products as well as pro-inflammatory cytokines and thereby initiate a signaling cascade that ultimately results in the up-regulation of inflammatory cytokines such as TNFα, IL6, and interferons. Recently it has become apparent that self-generated ligands such as nucleic acids and products of inflammation such as high-mobility group protein B1 (HMGB1) and Advanced Glycated End-products (AGE) are ligands for Toll-like receptors (TLRs) which are key receptors of the innate immune system (O'Neill 2003, Kanzler et al., 2007, Wagner 2006). This demonstrates the role of TLRs in the initiation and perpetuation of inflammation due to autoimmunity.

Interleukin-1 receptor associated kinase 4 (IRAK4) is a ubiquitously expressed serine/threonine kinase involved in the regulation of innate immunity (Suzuki & Saito 2006). IRAK4 is responsible for initiating signaling from TLRs and members of the IL-1/18 receptor family. Kinase-inactive knock-ins and targeted deletions of IRAK4 in mice were reported to cause reductions in TLR and IL-1 induced pro-inflammatory cytokines (Kawagoe et al., 2007; Fraczek et al., 2008; Kim et al., 2007). IRAK4 kinase-dead knock-in mice have also been shown to be resistant to induced joint inflammation in the antigen-induced-arthritis (AIA) and serum transfer-induced (K/BxN) arthritis models (Koziczak-Holbro 2009). Likewise, humans deficient in IRAK4 also appear to display the inability to respond to challenge by Toll ligands and IL-1 (Hernandez & Bastian 2006). However, the immunodeficient phenotype of IRAK4-null individuals is narrowly restricted to challenge by gram positive bacteria, but not gram negative bacteria, viruses or fungi. This gram positive sensitivity also lessens with age, implying redundant or compensating mechanisms for innate immunity in the absence of IRAK4 (Lavine et al., 2007).

These data indicate that inhibitors of IRAK4 kinase activity should have therapeutic value in treating cytokine driven autoimmune diseases while having minimal immunosuppressive side effects. Additional recent studies suggest that targeting IRAK4 may be useful in other inflammatory pathologies such as atherosclerosis and diffuse large B-cell lymphoma (Rekhter et al., 2008; Ngo et al., 2011). Therefore, inhibitors of IRAK4 kinase activity are potential therapeutics for a wide variety of diseases including but not limited to autoimmunity, inflammation, cardiovascular diseases, cancer, and metabolic diseases. See the following references for additional information. N. Suzuki and T. Saito, Trends in Immunology, 2006, 27, 566. T. Kawagoe, S. Sato, A. Jung, M. Yamamoto, K. Matsui, H. Kato, S. Uematsu, O. Takeuchi and S. Akira, Journal of Experimental Medicine, 2007, 204, 1013. J. Fraczek, T. W. Kim, H. Xiao, J. Yao, Q. Wen, Y. Li, J.-L. Casanova, J. Pryjma and X. Li, Journal of Biological Chemistry, 2008, 283, 31697. T. W. Kim, K. Staschke, K. Bulek, J. Yao, K. Peters, K.-H. Oh, Y. Vandenburg, H. Xiao, W. Qian, T. Hamilton, B. Min, G. Sen, R. Gilmour and X. Li, Journal of Experimental Medicine, 2007, 204, 1025. M. Koziczak-Holbro, A. Littlewood-Evans, B. Pollinger, J. Kovarik, J. Dawson, G. Zenke, C. Burkhart, M. Muller and H. Gram, Arthritis & Rheumatism, 2009, 60, 1661. M. Hernandez and J. F. Bastian, Current Allergy and Asthma Reports, 2006, 6, 468. E. Lavine, R. Somech, J. Y. Zhang, A. Puel, X. Bossuyt, C. Picard, J. L. Casanova and C. M. Roifman, Journal of Allergy and Clinical Immunology, 2007, 120, 948. M. Rekhter, K. Staschke, T. Estridge, P. Rutherford, N. Jackson, D. Gifford-Moore, P. Foxworthy, C. Reidy, X.-d. Huang, M. Kalbfleisch, K. Hui, M.-S. Kuo, R. Gilmour and C. J. Vlahos, Biochemical and Biophysical Research Communications, 2008, 367, 642. O'Neill, L. A. (2003). “Therapeutic targeting of Toll-like receptors for inflammatory and infectious diseases.” Curr Opin Pharmacol 3(4): 396. Kanzler, H et al., (2007) “Therapeutic targeting of innate immunity with toll-like receptor agonists and antagonists.” Nature Medicine 13:552. Wagner, H. (2006) “Endogenous TLR ligands and autoimmunity” Advances in Immunol 91: 159. Ngo, V. N. et al. (2011) “Oncogenically active MyD88 mutations in human lymphoma” Nature 470: 115.

Co-pending U.S. patent application Ser. No. 14/678,114, filed by Pfizer Inc on Apr. 3, 2015, describes IRAK4 inhibitors and is hereby incorporated by reference in its entirety.

### SUMMARY OF THE INVENTION

The invention provides for compounds of the Formula Ia,

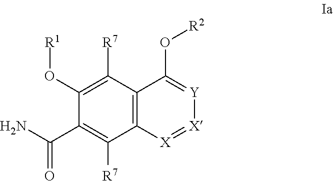

wherein

X and X′ are each independently CR⁶, N or —N⁺—O⁻; Y is independently N, —N⁺—O⁻ or CH; provided that at least one of X, X′ or Y is neither N nor —N⁺—O⁻ and that no more than one of X, X′ or Y is —N⁺—O⁻;

R¹ is C₁-C₆ alkyl or 3- to 7-membered cycloalkyl; wherein said alkyl or cycloalkyl is optionally substituted with one to five halogen, deutero, —OR⁵, —SR⁵, —NR^{11a}R^{11b}, cyano, C₁-C₆alkyl, C₃-C₆cycloalkyl or —C₁-C₆alkoxy;

R² is 3- to 10-membered cycloalkyl; 3- to 10-membered heterocycloalkyl having one to three heteroatoms; 5- to 10-membered heteroaryl having one to three heteroatoms; or C₆-C₁₂ aryl; wherein said cycloalkyl, heterocycloalkyl, heteroaryl or aryl is optionally substituted with one to five R³ and wherein, if the heteroatom on said heterocycloalkyl or heteroaryl is N, said N is optionally substituted with R⁴;

R³ for each occurrence is independently deuterium, halogen, C₁-C₆alkyl, C₃-C₆cycloalkyl, C₁-C₆alkoxy, oxo, —SR⁵, —NR^{11a}R^{11b}, cyano, or —OR⁵, wherein said alkyl, cycloalkyl or alkoxy is optionally and independently substituted with one to five deuterium, halogen, OR⁵, —SR⁵, —NR^{11a}R^{11b}, cyano, C₁-C₆alkyl, C₃-C₆cycloalkyl or C₁-C₆alkoxy; or two R³ taken together with the respective carbons to which each are bonded form a 3- to 6-membered cycloalkyl or a 4- to 6-membered heterocycloalkyl, wherein said cycloalkyl or heterocycloalkyl is optionally substituted with one to three halogen, deuterium, —OR⁵, —SR⁵, —NR^{11a}R^{11b}, cyano or C₁-C₆alkyl or C₁-C₆alkoxy, wherein the alkyl or alkoxy is optionally substituted with halogen, deuterium, —OR⁵, —SR⁵, —NR^{11a}R^{11b} or cyano; and wherein, if a heteroatom on said heterocycloalkyl is N, said N is optionally substituted with R⁴;

R⁴ is hydrogen, C₁-C₆ alkyl, —C(O)R¹⁰ or —S(O)₂R⁸, wherein the alkyl is optionally substituted with OH, halogen, deuterium, C₁-C₆alkyl, C₁-C₆alkoxy or cyano;

R⁵ is hydrogen or C₁-C₆alkyl, wherein said alkyl is optionally substituted with halogen, deuterium, C₁-C₆alkoxy, C₁-C₆alkylthiolyl, —NR^{11a}R^{11b}, cyano, C₁-C₆alkyl or C₃-C₆cycloalkyl;
each R⁶ is hydrogen, halogen, cyano, —OR⁵, —SR⁵, —NR^{11a}R^{11b}, C₁-C₆alkyl, C₃-C₆cycloalkyl, 3- to 7-membered heterocycloalkyl or 5- to 6-membered heteroaryl or aryl, wherein said alkyl, cycloalkyl, heterocycloalkyl, heteroaryl or aryl is optionally substituted with one to three halogen, —NR^{11a}R^{11b}, —OR⁵, —SR⁵, cyano, C₁-C₀₃ alkyl, —C(O)R¹⁰ or oxo;

R⁷ is independently hydrogen, methyl, cyano, OCF₃, OMe, CF₃ or halogen;

R⁸ is independently C₁-C₆alkyl, 3- to 6-membered cycloalkyl, 4- to 6-membered heterocycloalkyl, C₆-C₁₀aryl or 5- to 10-membered heteroaryl; wherein said alkyl, cycloalkyl, heterocycloalkyl, aryl or heteroaryl is optionally substituted with one to three deuterium, halogen, OH, C₁-C₆alkoxy, C₁-C₃alkyl (optionally substituted with NR^{11a}R^{11b} or C₁-C₆alkoxy), 3- to 6-membered cycloalkyl, NR^{11a}R^{11b} or cyano;

R¹⁰ is C₁-C₆alkyl, 3- to 6-membered cycloalkyl, 4- to 6-membered heterocycloalkyl, C₆-C₁₀aryl or 5- to 10-membered heteroaryl; wherein said alkyl, cycloalkyl, heterocycloalkyl, aryl or heteroaryl is optionally substituted with one to three deuterium, halogen, OH, C₁-C₆alkoxy, C₁-C₃alkyl (optionally substituted with NR^{11a}R^{11b} or C₁-C₆alkoxy), 3- to 6-membered cycloalkyl, NR^{11a}R^{11b} or cyano; and

R^{11a} and R^{11b} are each independently hydrogen, 3- to 6-membered cycloalkyl or C₁-C₆alkyl, wherein said cycloalkyl or alkyl is optionally substituted with deuterium, C₁-C₆alkoxy or cyano; and if said alkyl is C₂-C₆alkyl, said alkyl is optionally substituted with deuterium, C₁-C₆alkoxy, cyano, halogen or OH;

or a pharmaceutically acceptable salt of said compound or a tautomer of said compound or said salt.

The invention also provides for pharmaceutical compositions comprising the compounds, methods of using the compounds, combination therapies utilizing the compounds and other therapeutic agents and methods of preparing the compounds. The invention also provides for intermediates useful in the preparation of the compounds of the invention.

In particular, novel bicyclic kinase enzyme inhibitor compounds of Formula Ia of the present invention possess a therapeutic role of inhibiting IRAK4 useful in the area of diseases and/or disorders that include, but are not limited to, cancers, allergic diseases, autoimmune diseases, inflammatory diseases and/or disorders and/or conditions associated with inflammation and pain, proliferative diseases, hematopoietic disorders, hematological malignancies, bone disorders, fibrosis diseases and/or disorders, metabolic disorders, muscle diseases and/or disorders, respiratory diseases, pulmonary disorders, genetic development diseases, neurological and neurodegenerative diseases and/or disorders, chronic inflammatory demyelinating neuropathies, cardiovascular, vascular or heart diseases, ophthalmic/ocular diseases, wound repair, infection and viral diseases. Therefore, inhibition of IRAK4 would have the potential for multiple therapeutic indications over a wide range of unmet needs.

### DETAILED DESCRIPTION OF THE INVENTION

The present invention may be understood more readily by reference to the following detailed description of exemplary embodiments of the invention and the examples included therein. It is to be understood that this invention is not limited to specific methods of synthesis, which may of course vary. It is also to be understood that the terminology used herein is for the purpose of describing particular embodiments only and is not intended to be limiting.

All patents, patent applications and references referred to herein are hereby incorporated by reference in their entirety.

Other features and advantages of this invention will be apparent from this specification and the appendent claims which describe the invention. There are many features of this invention that are not necessarily fully captured by the claims. It is understood, however, that all such novel subject matter is part of the invention.

### Definitions

Unless otherwise defined herein, scientific and technical terms used in connection with the present invention have the meaning commonly understood by those of ordinary skill in the art. As used in the specification and the appended claims, the singular forms “a”, “an”, and “the” include plural referents unless the context clearly dictates otherwise.

The term “about” refers to a relative term denoting an approximation of plus or minus 10% of the nominal value it refers, in one embodiment, to plus or minus 5%, in another embodiment, to plus or minus 2%. For the field of this disclosure, this level of approximation is appropriate unless the value is specifically stated require a tighter range.

The term “alkyl” refers to a linear or branched saturated hydrocarbon moiety, consisting solely of carbon and hydrogen atoms. In one embodiment from one to six carbon atoms; and in another embodiment from one to four carbon atoms; and in another embodiment one to three carbon atoms. Non-limiting examples of such substituents include methyl, ethyl, propyl (including n-propyl and isopropyl), butyl (including n-butyl, isobutyl, sec-butyl and tert-butyl), pentyl, isoamyl, hexyl and the like. As appropriate, an alkyl may be optionally substituted at each carbon as defined in the claims. Typical substitution includes, but is not limited to, fluoro, chloro, OH, cyano, alkyl (optionally substituted), cycloalkyl and the like.

In some instances, the number of carbon atoms in a hydrocarbon substituent (i.e., alkyl, cycloalkyl, etc.) is indicated by the prefix “Cₓ-C_{y}-” or “C_{x-y}”, wherein x is the minimum and y is the maximum number of carbon atoms in the substituent. Thus, for example, “C₁-C₆-alkyl” or “C₁₋₆ alkyl” refers to an alkyl substituent containing from 1 to 6 carbon atoms. Illustrating further, C₃-C₆-cycloalkyl or C₃₋₆-cycloalkyl refers to saturated cycloalkyl containing from 3 to 6 carbon ring atoms.

Unless otherwise indicated, “alkylene,” by itself or as part of another term, refers to a saturated, branched or straight chain or cyclic hydrocarbon diradical of the stated number of carbon atoms, typically 1-6 carbon atoms, and having two monovalent radical centers derived by the removal of two hydrogen atoms from the same or two different carbon atoms of a parent alkane. Typical alkylene radicals include, but are not limited to methylene (—CH₂—), 1,2-ethylene (—CH₂CH₂—), 2,2-dimethylene, 1,3-propylene (—CH₂CH₂CH₂—), 2-methylpropylene, 1,4-butylene (—CH₂CH₂CH₂CH₂—), and the like; optionally substituted, as appropriate, by 1 to 5 suitable substituents as defined above such as fluoro, chloro, deutero, cyano, trifluoromethyl, (C₁-C₆)alkoxy, (C₆-C₁₀)aryloxy, trifluoromethoxy, difluoromethoxy or (C₁-C₆)alkyl. When the compounds of the invention contain a C₂₋₆alkenyl group, the compound may exist as the pure E (entgegen) form, the pure Z (zusammen) form, or any mixture thereof.

“Alkylidene” or “alkenyl” refers to a divalent group formed from an alkane by removal of two hydrogen atoms from the same carbon atom, the free valencies of which are part of a double bond, optionally substituted as described herein. The term alkylidene also includes “allenes” wherein on carbon atom has double bonds with each of its two adjacent carbon centers, such as, for example, propadiene. As appropriate, an alkenyl may be optionally substituted at each carbon as defined in the claims, optionally substituted, as appropriate, by 1 to 5 suitable substituents as defined above and herein such as fluoro, chloro, deutero, cyano, trifluoromethyl, (C₁-C₆)alkoxy, (C₆-C₁₀)aryloxy, trifluoromethoxy, difluoromethoxy or (C₁-C₆)alkyl.

“Alkynyl” refers to an aliphatic hydrocarbon having at least one carbon-carbon triple bond, including straight chain, branched chain or cyclic groups having at least one carbon-carbon triple bond, optionally substituted as described herein. Preferably, it is a lower alkynyl having 2 to 6 carbon atoms. For example, as used herein, the term “C₂₋₆alkynyl” is used herein to mean a straight or branched hydrocarbon chain alkynyl radical as defined above having 2 to 6 carbon atoms and one triple bond. As appropriate, an alkynyl may be optionally substituted at each carbon as defined in the claims. Typical substitution includes, but is not limited to, optionally substituted, as appropriate, by 1 to 5 suitable substituents as defined above and herein, such as fluoro, chloro, deutero, cyano, trifluoromethyl, (C₁-C₆)alkoxy, (C₆-C₁₀)aryloxy, trifluoromethoxy, difluoromethoxy or (C₁-C₆)alkyl.

The term “cycloalkyl” refers to a nonaromatic ring containing 3 to 10 carbons that is fully hydrogenated consisting of mono-, bi- or tricyclic rings. Accordingly, a cycloalkyl may be a single ring, which typically contains from 3 to 7 ring atoms. Examples include, but are not limited to, cyclopropyl, cyclobutyl, cyclopentyl, and cyclohexyl. Alternatively, 2 or 3 rings may be fused together, such as bicyclodecanyl and decalinyl. The term “cycloalkyl” also includes bridged bicycloalkyl systems such as, but not limited to, bicyclo[2.2.1]heptane and bicyclo[1.1.1]pentane. The cycloalkyl group may be optionally substituted as described herein, as appropriate, by 1 to 5 suitable substituents as defined above such as fluoro, chloro, deutero, cyano, trifluoromethyl, (C₁-C₆)alkoxy, (C₆-C₁₀)aryloxy, trifluoromethoxy, difluoromethoxy or (C₁-C₆)alkyl.

The term “heterocycloalkyl” means a monovalent saturated moiety, consisting of one to three rings, incorporating one, two, three or four heteroatoms (selected from N, O or S) and three to 10 carbon atoms. The heterocycloalkyl may be optionally substituted as defined herein. Examples of heterocycloalkyl moieties include, but are not limited to, optionally substituted piperidinyl, piperazinyl, homopiperazinyl, azepinyl, pyrrolidinyl, pyrazolidinyl, imidazolinyl, imidazolidinyl, pyridinyl, pyridazinyl, pyrimidinyl, oxazolidinyl, isoxazolidinyl, morpholinyl, thiazolidinyl, isothiazolidinyl, quinuclidinyl, quinolinyl, isoquinolinyl, benzimidazolyl, thiadiazolylidinyl, benzothiazolidinyl, benzoazolylidinyl, dihydrofuryl, tetrahydrofuryl, dihydropyranyl, tetrahydropyranyl, thiamorpholinyl, thiamorpholinylsulfoxide, thiamorphilinylsulfone, dihydroquinolinyl, tetrahydroquinolinyl, tetrahydrisoquinolinyl, and the like. Heterocycloalkyls may be optionally substituted, as appropriate, by 1 to 5 suitable substituents as defined herein such as fluoro, chloro, deutero, cyano, trifluoromethyl, (C₁-C₆)alkoxy, (C₆-C₁₀)aryloxy, trifluoromethoxy, difluoromethoxy or (C₁-C₆)alkyl.

Unless otherwise indicated, the term “heteroalkyl,” by itself or in combination with another term, means, unless otherwise stated, a saturated, straight or branched chain hydrocarbon radical consisting of the stated number of carbon atoms and from one to three heteroatoms selected from the group consisting of O, N and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom S may be placed at any position of the heteroalkyl group, including the position at which the alkyl group is attached to the remainder of the molecule. Up to two heteroatoms may be consecutive.

Unless otherwise indicated, the term “heteroalkylene” by itself or as part of another substituent means a divalent group derived from heteroalkyl (as defined above). For heteroalkylene groups, heteroatoms can also occupy either or both of the chain termini.

The term “alkoxy” and “alkyloxy”, which may be used interchangeably, refers to a moiety of the formula —OR, wherein R is a straight chain saturated alkyl or branched chain saturated alkyl moiety, as defined herein, bonded through an oxygen atom. The alkoxy group may be optionally substituted as defined herein. Non-limiting examples of such alkoxy groups are methoxy, ethoxy, propoxy, isopropoxy, butoxy, isobutoxy, tertiary butoxy, pentoxy and the like.

The term “aryl” means a carbocyclic aromatic system containing one or two rings wherein such rings may be fused. If the rings are fused, one of the rings must be fully unsaturated and the fused ring(s) may be fully saturated, partially unsaturated or fully unsaturated. The term “fused” means that a second ring is present (i.e., attached or formed) by having two adjacent atoms in common (i.e., shared) with the first ring. The term “fused” is equivalent to the term “condensed”. The aryl group may be optionally substituted as defined herein. The term “aryl” embraces aromatic radicals such as phenyl, naphthyl, tetrahydronaphthyl, indanyl, biphenyl, benzo[b][1,4]oxazin-3(4H)-onyl, 2,3-dihydro-1H indenyl and 1,2,3,4-tetrahydronaphthalenyl. Aryls may be optionally substituted, as appropriate, by 1 to 5 suitable substituents as defined above such as fluoro, chloro, deutero, cyano, trifluoromethyl, (C₁-C₆)alkoxy, (C₆-C₁₀)aryloxy, trifluoromethoxy, difluoromethoxy or (C₁-C₆)alkyl.

The term “heteroaryl” refers to an aromatic ring structure containing from 5 to 6 ring atoms in which at least one of the ring atoms is a heteroatom (i.e., oxygen, nitrogen, or sulfur), with the remaining ring atoms being independently selected from the group consisting of carbon, oxygen, nitrogen, and sulfur. Examples of heteroaryl substituents include 6-membered ring substituents such as pyridyl, pyrazyl, pyrimidinyl, and pyridazinyl; and 5-membered ring substituents such as triazolyl, imidazolyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl. In a group that has a heteroaryl substituent, the ring atom of the heteroaryl substituent that is bound to the group may be one of the heteroatoms, or it may be a ring carbon atom. Similarly, if the heteroaryl substituent is in turn substituted with a group or substituent, the group or substituent may be bound to one of the heteroatoms, or it may be bound to a ring carbon atom. The term “heteroaryl” also includes pyridyl N-oxides and groups containing a pyridine N-oxide ring.

Further examples include furyl, thienyl, oxazolyl, thiazolyl, imidazolyl, pyrazolyl, triazolyl, tetrazolyl, isoxazolyl, isothiazolyl, oxadiazolyl, thiadiazolyl, pyridinyl, pyridazinyl, pyrimidinyl, pyrazinyl, pyridin-2(1H)-onyl, pyridazin-2(1H)-onyl, pyrimidin-2(1H)-onyl, pyrazin-2(1H)-onyl, imidazo[1,2-a]pyridinyl, pyrazolo[1,5-a]pyridinyl, 5,6,7,8-tetrahydroisoquinolinyl, 5,6,7,8-tetrahydroquinolinyl, 6,7-dihydro-5H-cyclopenta[b]pyridinyl, 6,7-dihydro-5H-cyclopenta[c]pyridinyl, 1,4,5,6-tetrahydrocyclopenta[c]pyrazolyl, 2,4,5,6-tetrahydrocyclopenta[c]pyrazolyl, 5,6-dihydro-4H-pyrrolo[1,2-b]pyrazolyl, 6,7-dihydro-5H-pyrrolo[1,2-b][1,2,4]triazolyl, 5,6,7,8-tetrahydro-[1,2,4]triazolo[1,5-a]pyridinyl, 4,5,6,7-tetrahydropyrazolo[1,5-a]pyridinyl, 4,5,6,7-tetrahydro-1H-indazolyl and 4,5,6,7-tetrahydro-2H-indazolyl. The heteroaryl can be optionally substituted, as appropriate, by 1 to 5 suitable substituents as defined herein such as fluoro, chloro, deutero, cyano, trifluoromethyl, (C₁-C₆)alkoxy, (C₆-C₁₀)aryloxy, trifluoromethoxy, difluoromethoxy or (C₁-C₆)alkyl.

Examples of single-ring heteroaryls and heterocycloalkyls include furanyl, dihydrofuranyl, tetrahydrofuranyl, thiophenyl, dihydrothiophenyl, tetrahydrothiophenyl, pyrrolyl, isopyrrolyl, pyrrolinyl, pyrrolidinyl, imidazolyl, isoimidazolyl, imidazolinyl, imidazolidinyl, pyrazolyl, pyrazolinyl, pyrazolidinyl, triazolyl, tetrazolyl, dithiolyl, oxathiolyl, oxazolyl, isoxazolyl, thiazolyl, isothiazolyl, thiazolinyl, isothiazolinyl, thiazolidinyl, isothiazolidinyl, thiaoxadiazolyl, oxathiazolyl, oxadiazolyl (including oxadiazolyl, 1,2,4-oxadiazolyl, 1,2,5-oxadiazolyl, or 1,3,4-oxadiazolyl), pyranyl (including 1,2-pyranyl or 1,4-pyranyl), dihydropyranyl, pyridinyl, piperidinyl, diazinyl (including pyridazinyl, pyrimidinyl, piperazinyl), triazinyl (including s-triazinyl, as-triazinyl and v-triazinyl), oxazinyl (including 2H-1,2-oxazinyl, 6H-1,3-oxazinyl, or 2H-1,4-oxazinyl), isoxazinyl (including o-isoxazinyl or p-isoxazinyl), oxazolidinyl, isoxazolidinyl, oxathiazinyl (including 1,2,5-oxathiazinyl or 1,2,6-oxathiazinyl), oxadiazinyl (including 2H-1,2,4-oxadiazinyl or 2H-1,2,5-oxadiazinyl), and morpholinyl.

The term “heteroaryl” also includes fused ring systems having one or two rings wherein such rings may be fused, wherein fused is as defined above. It is to be understood that if a carbocyclic or heterocyclic moiety may be bonded or otherwise attached to a designated substrate through differing ring atoms without denoting a specific point of attachment, then all possible points are intended, whether through a carbon atom or, for example, a trivalent nitrogen atom. For example, the term “pyridyl” means 2-, 3- or 4-pyridyl, the term “thienyl” means 2- or 3-thienyl, and so forth.

In some instances, the number of atoms in a cyclic substituent containing one or more heteroatoms (i.e., heteroaryl or heterocycloalkyl) is indicated by the prefix “x- to y-membered”, wherein x is the minimum and y is the maximum number of atoms forming the cyclic moiety of the substituent. Thus, for example, “5- to 6-membered heteroaryl” refers to a heteroaryl containing from 5 to 6 atoms, including one or more heteroatoms, in the cyclic moiety of the heteroaryl. The heteroatoms for this invention are selected from nitrogen, oxygen and sulfur.

Compounds of the present invention may contain basic nitrogen atoms (e.g. alkyl amines or heterocycles such as pyridine etc.) which may be converted to N-oxides by treatment with an oxidizing agent (e.g. MCPBA and/or hydrogen peroxides) to afford other compounds of this invention. Thus, all nitrogen-containing compounds that may converted to N-oxide (N→O or —N+-O—) derivatives are part of the invention.

One skilled in the art would appreciate that metabolites may be formed as part of the natural biochemical process of degrading and eliminating the compounds. For example, some compounds of the invention may naturally form an N-oxide, as depicted below in the compound of Formula IIIa and IIIb or in other areas of the compound of Formula Ia. Metabolites such as these or others formed as part of the natural biochemical process are within the scope of the invention.

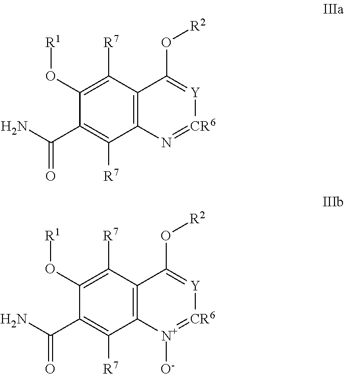

If substituents are described as “independently” having more than one variable, each instance of a substituent is selected independent of the other from the list of variables available. Each substituent therefore may be identical to or different from the other substituent(s).

“Patient” or “subject” refers to warm-blooded animals such as, for example, guinea pigs, mice, rats, gerbils, cats, rabbits, dogs, cattle, goats, sheep, horses, monkeys, chimpanzees, and humans.

The term “pharmaceutically acceptable” means the substance or composition must be compatible, chemically and/or toxicologically, with the other ingredients comprising a formulation, and/or the mammal being treated therewith.

The term “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “treating”, as used herein, unless otherwise indicated, means reversing, alleviating, inhibiting the progress of, delaying the progression of, delaying the onset of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject. For the avoidance of doubt, reference herein to “treatment” includes reference to curative, palliative and prophylactic treatment, and to the administration of a medicament for use in such treatment.

As used herein, the terms “Formula I”, “Formula Ia”, “Formula IIa-IIg”, “Formula IIIa” and “Formula IIIb” may be hereinafter referred to as a “compound(s) of the invention,” “the present invention,” and collectively the “compound of Formula I.” Accordingly, the term “compound of Formula I” includes the compounds of Formula Ia, IIa-IIg, IIIa and IIIb. Such terms are also defined to include all forms of the compound of Formula I, including hydrates, solvates, isomers, crystalline and non-crystalline forms, isomorphs, polymorphs, tautomers and metabolites thereof. For example, the compounds of the invention, or pharmaceutically acceptable salts thereof, may exist in unsolvated and solvated forms. When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

The compounds of the invention have asymmetric carbon atoms. The carbon-carbon bonds of the compounds of the invention may be depicted herein using a solid line (-), a solid wedge ( [symbol] ), or a dotted wedge ( [symbol] ). The use of a solid line to depict bonds to asymmetric carbon atoms is meant to indicate that all possible stereoisomers (e.g., specific enantiomers, racemic mixtures, etc.) at that carbon atom are included. The use of either a solid or dotted wedge to depict bonds to asymmetric carbon atoms is meant to indicate that only the stereoisomer shown is meant to be included. It is possible that compounds of Formula Ia may contain more than one asymmetric carbon atom. In those compounds, the use of a solid line to depict bonds to asymmetric carbon atoms is meant to indicate that all possible stereoisomers are meant to be included. For example, unless stated otherwise, it is intended that the compounds of Formula Ia can exist as enantiomers and diastereomers or as racemates and mixtures thereof. The use of a solid line to depict bonds to one or more asymmetric carbon atoms in a compound of Formula Ia and the use of a solid or dotted wedge to depict bonds to other asymmetric carbon atoms in the same compound is meant to indicate that a mixture of diastereomers is present.

Stereoisomers of Formula Ia include cis and trans isomers, optical isomers such as R and S enantiomers, diastereomers, geometric isomers, rotational isomers, conformational isomers, and tautomers of the compounds of the invention, including compounds exhibiting more than one type of isomerism; and mixtures thereof (such as racemates and diastereomeric pairs). Also included are acid addition or base addition salts wherein the counterion is optically active, for example, D-lactate or L-lysine, or racemic, for example, DL-tartrate or DL-arginine.

Some of the compounds of the invention, such as 23, 27 and 66, may exhibit the phenomenon of tautomerism. For example, the compound exemplified by 23 may exist in several tautomeric forms, including the pyrrolidin-2-one form, Example 23, and the 5-hydroxy-3,4-dihydro-2H-pyrrol form, Example 23a. All such tautomeric forms are included within the scope of the compounds of the Formula Ia and the scope of the invention. One of ordinary skill in the art would appreciate and recognize that many of the Examples described herein may exhibit tautomerism and are within the scope of the compound of Formula Ia, IIa-IIg, IIIa and IIIb, such terms being used interchangeably. Tautomers exist as mixtures of a tautomeric set in solution. In solid form, usually one tautomer predominates. Even though one tautomer may be described, the present invention includes all tautomers of the compounds of the invention and salts thereof. Examples of tautomers are described by Examples 23 and 23a.

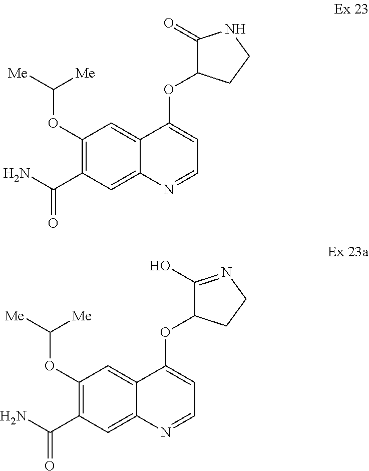 [SMILES: CC(C)Oc1cc2c(OC3CCNC3=O)ccnc2cc1C(N)=O ; CC(C)Oc1cc2c(OC3CCN=C3O)ccnc2cc1C(N)=O]

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

The compounds of this invention may be used in the form of salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also may be used as an aid in the isolation, purification, and/or resolution of the compound.

Where a salt is intended to be administered to a patient (as opposed to, for example, being used in an in vitro context), the salt preferably is pharmaceutically acceptable. The term “pharmaceutically acceptable salt” refers to a salt prepared by combining a compound of Formula Ia with an acid whose anion, or a base whose cation, is generally considered suitable for human consumption. Pharmaceutically acceptable salts are particularly useful as products of the methods of the present invention because of their greater aqueous solubility relative to the parent compound. For use in medicine, the salts of the compounds of this invention are non-toxic “pharmaceutically acceptable salts.” Salts encompassed within the term “pharmaceutically acceptable salts” refer to non-toxic salts of the compounds of this invention, which are generally prepared by reacting the free base with a suitable organic or inorganic acid.

Suitable pharmaceutically acceptable acid addition salts of the compounds of the present invention when possible include those derived from inorganic acids, such as hydrochloric, hydrobromic, hydrofluoric, boric, fluoroboric, phosphoric, metaphosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. Suitable organic acids generally include, for example, aliphatic, cycloaliphatic, aromatic, araliphatic, heterocyclic, carboxylic, and sulfonic classes of organic acids.

Specific examples of suitable organic acids include acetate, trifluoroacetate, formate, propionate, succinate, glycolate, gluconate, digluconate, lactate, malate, tartrate, citrate, ascorbate, glucuronate, maleate, fumarate, pyruvate, aspartate, glutamate, benzoate, anthranilate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sulfanilate, cyclohexylaminosulfonate, algenate, β-hydroxybutyrate, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, and undecanoate.

Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. In another embodiment, base salts are formed from bases which form non-toxic salts, including aluminum, arginine, benzathine, choline, diethylamine, diolamine, glycine, lysine, meglumine, olamine, tromethamine and zinc salts.

Organic salts may be made from secondary, tertiary or quaternary amine salts, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups may be quaternized with agents such as lower alkyl (C₁-C₆) halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl, and stearyl chlorides, bromides, and iodides), arylalkyl halides (e.g., benzyl and phenethyl bromides), and others.

In one embodiment, hemisalts of acids and bases may also be formed, for example, hemisulfate and hemicalcium salts.

Also within the scope of the present invention are so-called “prodrugs” of the compound of the invention. Thus, certain derivatives of the compound of the invention that may have little or no pharmacological activity themselves can, when administered into or onto the body, be converted into the compound of the invention having the desired activity, for example, by hydrolytic cleavage. Such derivatives are referred to as “prodrugs.” Further information on the use of prodrugs may be found in “Pro-drugs as Novel Delivery Systems, Vol. 14, ACS Symposium Series (T. Higuchi and V. Stella) and “Bioreversible Carriers in Drug Design,” Pergamon Press, 1987 (ed. E. B. Roche, American Pharmaceutical Association). Prodrugs in accordance with the invention can, for example, be produced by replacing appropriate functionalities present in the compounds of any of Formula Ia with certain moieties known to those skilled in the art as “pro-moieties” as described, for example, in “Design of Prodrugs” by H. Bundgaard (Elsevier, 1985).

The present invention also includes isotopically labeled compounds, which are identical to those recited in Formula Ia, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, sulfur, fluorine and chlorine, such as ²H, ³H, ¹³C, ¹¹C, ¹⁴C ¹⁵N, ¹⁸O, ¹⁷O, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Compounds of the invention as claimed in the claims may specifically define substitution with deutero or deuterium. The absence of the term deuteron, deuteron or deuterium, all of which are used interchangeably, in a substitution group shall not be implied to exclude deutero.

Isotopically labeled compounds of Formula Ia of this invention and prodrugs thereof can generally be prepared by carrying out the procedures disclosed in the Schemes and/or in the Examples and Preparations below, by substituting a readily available isotopically labeled reagent for a non-isotopically labeled reagent.

All patents and publications identified herein are incorporated herein by reference in their entirety and for all purposes.

### Compounds of the Invention

In one embodiment, as described above and more fully herein, the invention is directed to a compound of Formula Ia,

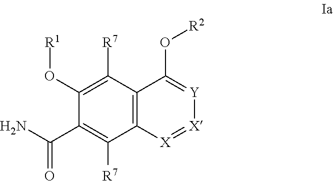

wherein

X and X′ are each independently CR⁶, N or —N⁺—O⁻; Y is independently N, —N⁺—O⁻ or CH; provided that at least one of X, X′ or Y is neither N nor —N⁺—O⁻ and that no more than one of X, X′ or Y is —N⁺—O⁻;

R¹ is C₁-C₆ alkyl or 3- to 7-membered cycloalkyl; wherein said alkyl or cycloalkyl is optionally substituted with one to five halogen, deutero, —OR⁵, —SR⁵, —NR^{11a}R^{11b}, cyano, C₁-C₆alkyl, C₃-C₆cycloalkyl or —C₁-C₆alkoxy;

R² is 3- to 10-membered cycloalkyl; 3- to 10-membered heterocycloalkyl, having one to three heteroatoms; 5- to 10-membered heteroaryl having one to three heteroatoms; or C₆-C₁₂ aryl; wherein said cycloalkyl, heterocycloalkyl, heteroaryl or aryl is optionally substituted with one to five R³ and wherein, if the heteroatom on said heterocycloalkyl or heteroaryl is N, said N is optionally substituted with R⁴;

R³ for each occurrence is independently deuterium, halogen, C₁-C₆alkyl, C₃-C₆cycloalkyl, C₁-C₆alkoxy, oxo, —SR⁵, —NR^{11a}R^{11b}, cyano, or —OR⁵, wherein said alkyl, cycloalkyl or alkoxy is optionally and independently substituted with one to five deuterium, halogen, OR⁵, —SR⁵, —NR^{11a}R^{11b}, cyano, C₁-C₆alkyl, C₃-C₆cycloalkyl or C₁-C₆alkoxy; or two R³ taken together with the respective carbons to which each are bonded form a 3- to 6-membered cycloalkyl or a 4- to 6-membered heterocycloalkyl, wherein said cycloalkyl or heterocycloalkyl is optionally substituted with one to three halogen, deuterium, —OR⁵, —SR⁵, —NR^{11a}R^{11b}, cyano or C₁-C₆alkyl or C₁-C₆alkoxy, wherein the alkyl or alkoxy is optionally substituted with halogen, deuterium, —OR⁵, —SR⁵, —NR^{11a}R^{11b} or cyano; and wherein, if a heteroatom on said heterocycloalkyl is N, said N is optionally substituted with R⁴;

R⁴ is hydrogen, C₁-C₆ alkyl, —C(O)R¹⁰ or —S(O)₂R⁸, wherein the alkyl is optionally substituted with OH, halogen, deuterium, C₁-C₆alkyl, C₁-C₆alkoxy or cyano;

R⁵ is hydrogen or C₁-C₆alkyl, wherein said alkyl is optionally substituted with halogen, deuterium, C₁-C₆alkoxy, C₁-C₆alkylthiolyl, —NR^{11a}R^{11b}, cyano, C₁-C₆alkyl or C₃-C₆cycloalkyl;

each R⁶ is hydrogen, halogen, cyano, —OR⁵, —SR⁵, —NR^{11a}R^{11b}, C₁-C₆alkyl, C₃-C₆cycloalkyl, 3- to 7-membered heterocycloalkyl or 5- to 6-membered heteroaryl or aryl, wherein said alkyl, cycloalkyl, heterocycloalkyl, heteroaryl or aryl is optionally substituted with one to three halogen, —NR^{11a}R^{11b}, —OR⁵, —SR⁵, cyano, C₁-C₃ alkyl, —C(O)R¹⁰ or oxo;

R⁷ is independently hydrogen, methyl, cyano, OCF₃, OMe, CF₃ or halogen;

R⁸ is independently C₁-C₆alkyl, 3- to 6-membered cycloalkyl, 4- to 6-membered heterocycloalkyl, C₆-C₁₀aryl or 5- to 10-membered heteroaryl; wherein said alkyl, cycloalkyl, heterocycloalkyl, aryl or heteroaryl is optionally substituted with one to three deuterium, halogen, OH, C₁-C₆alkoxy, C₁-C₃alkyl (optionally substituted with NR^{11a}R^{11b} or C₁-C₆alkoxy), 3- to 6-membered cycloalkyl, NR^{11a}R^{11b} or cyano;

R¹⁰ is C₁-C₆alkyl, 3- to 6-membered cycloalkyl, 4- to 6-membered heterocycloalkyl, C₆-C₁₀aryl or 5- to 10-membered heteroaryl; wherein said alkyl, cycloalkyl, heterocycloalkyl, aryl or heteroaryl is optionally substituted with one to three deuterium, halogen, OH, C₁-C₆alkoxy, C₁-C₃alkyl (optionally substituted with NR^{11a}R^{11b} or C₁-C₆alkoxy), 3- to 6-membered cycloalkyl, NR^{11a}R^{11b} or cyano; and

R^{11a} and R^{11b} are each independently hydrogen, 3- to 6-membered cycloalkyl or C₁-C₆alkyl, wherein said cycloalkyl or alkyl is optionally substituted with deuterium, C₁-C₆alkoxy or cyano; and if said alkyl is C₂-C₆alkyl, said alkyl is optionally substituted with deuterium, C₁-C₆alkoxy, cyano, halogen or OH;

or a pharmaceutically acceptable salt of said compound or a tautomer of said compound or said salt.

In one embodiment, X is N, X′ is CR⁶ and Y is CH; X is N, X′ is N and Y is CH; X is N, X′ is CR⁶ and Y is N; X is CR⁶, X′ and Y are N; X and X′ are CR⁶ and Y is N; X is CR⁶ and Y is CH and X′ is N; X and X′ are CR⁶ and Y is CH; or a pharmaceutically acceptable salt of said compound or a tautomer of said salt.

In another embodiment, the invention comprises a compound of Formula IIa, IIb, IIc, IId, IIe, IIf or IIg,

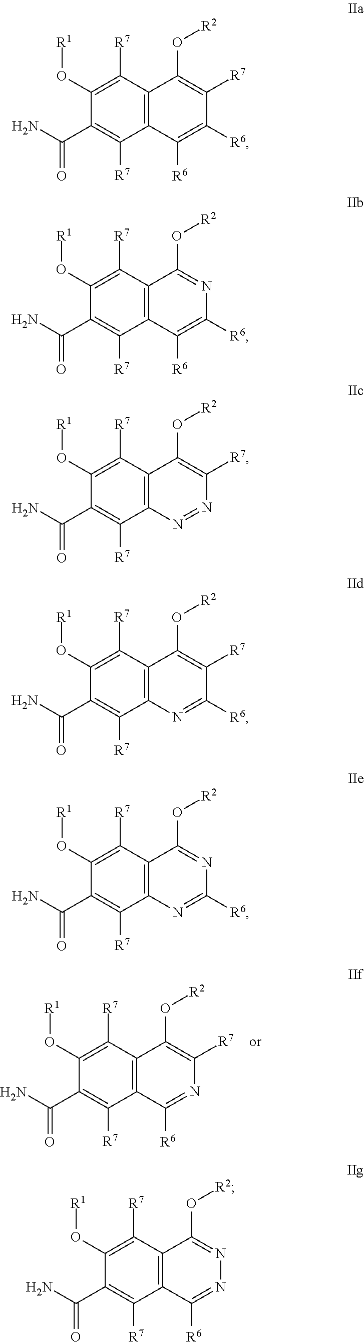

wherein

R¹ is C₁-C₆ alkyl or 3- to 7-membered cycloalkyl; wherein said alkyl or cycloalkyl is optionally substituted with one to five halogen, deutero, —OR⁵ or cyano;

R² is 3- to 7-membered cycloalkyl; 3- to 7-membered heterocycloalkyl, having one to three heteroatoms; 5- to 10 membered heteroaryl having one to three heteroatoms; or C₆-C₁₂ aryl; wherein said cycloalkyl, heterocycloalkyl, heteroaryl or aryl is optionally substituted with one to five R³ and wherein, if a heteroatom on said heterocycloalkyl or heteroaryl is N, said N is optionally substituted with R⁴;

R³ for each occurrence is independently deuterium, halogen, C₁-C₆alkyl, oxo, —OR⁵, wherein said alkyl, is optionally and independently substituted with one to five deuterium, halogen, OR⁵, —SR⁵, —NR^{11a}R^{11b}, cyano, C₁-C₆ alkyl, C₃-C₆ cycloalkyl or C₁-C₆ alkoxy; or two R³ taken together with the respective carbons to which each are bonded form a 3- to 6-membered cycloalkyl or a 4- to 6-membered heterocycloalkyl, wherein said cycloalkyl or heterocycloalkyl is optionally substituted with one to three halogen, deuterium, —OR⁵, —SR⁵, —NR^{11a}R^{11b}, cyano or C₁-C₆alkyl or C₁-C₆alkoxy, wherein the alkyl or alkoxy is optionally substituted with halogen, deuterium, —OR⁵, —SR⁵, —NR^{11a}R^{11b} or cyano; and wherein, if a heteroatom on said heterocycloalkyl is N, said N is optionally substituted with R⁴;

R⁴ is independently hydrogen, C₁-C₆ alkyl, —C(O)R¹⁰ or —S(O)₂R⁸, wherein the alkyl is optionally substituted with OH, halogen, deuterium, C₁-C₆alkyl, C₁-C₆alkoxy or cyano;

R⁵ is independently hydrogen or C₁-C₆alkyl, wherein said alkyl is optionally substituted with halogen, deuterium, C₁-C₆alkoxy, C₁-C₆alkylthiolyl, —NR^{11a}R^{11b}, cyano, C₁-C₆alkyl or C₃-C₆cycloalkyl;

each R⁶ is independently hydrogen, halogen, cyano, —OR⁵, —SR⁵, —NR^{11a}R^{11b}, C₁-C₆alkyl, C₃-C₆cycloalkyl, 3- to 10-membered heterocycloalkyl or 5- to 6-membered heteroaryl or aryl, wherein said alkyl, cycloalkyl, heterocycloalkyl, heteroaryl or aryl is optionally substituted with one to three halogen, —NR^{11a}R^{11b}, —OR⁵, —SR⁵, cyano, C₁-C₃ alkyl, —C(O)R¹⁰ or oxo;

R⁷ is independently hydrogen, methyl, CF₃ or halogen;

R⁸ is C₁-C₃alkyl, 3- to 6-membered cycloalkyl, 4- to 6-membered heteroaryl or C₆-C₁₀aryl, wherein said alkyl, cycloalkyl, heteroaryl or aryl are each optionally substituted with fluoro, 3-6-membered cycloalkyl or C₁-C₃alkyl;

R¹⁰ is C₁-C₆alkyl, 3- to 6-membered cycloalkyl, 4- to 6-membered heterocycloalkyl, C₆-C₁₀aryl or 5- to 6-membered heteroaryl; wherein said alkyl, cycloalkyl, heterocycloalkyl, aryl or heteroaryl is optionally substituted with one to three deuterium, halogen, OH, C₁-C₆alkoxy, C₁-C₃alkyl (optionally substituted with NR^{11a}R^{11b} or C₁-C₆alkoxy), 3- to 6-membered cycloalkyl, NR^{11a}R^{11b} or cyano;

R^{11a} and R^{11b} are each independently hydrogen, 3- to 6-membered cycloalkyl or C₁-C₆alkyl, wherein said cycloalkyl or alkyl is optionally substituted with deuterium, C₁-C₆alkoxy or cyano; and if said alkyl is C₂-C₆alkyl, said alkyl is optionally substituted with deuterium, C₁-C₆alkoxy, cyano, halogen or OH; or a pharmaceutically acceptable salt of said compound or a tautomer of said compound or said salt.

In another embodiment, R² is selected from pyrrolidinyl, pyrrolidin-2-onyl, piperidinyl, piperidin-2-onyl, octahydro-1H-pyrrolo[3,4-c]pyridinyl, oxazolidinyl, oxazolidin-2-onyl, 1,3-oxazinan-2-onyl, imidazolidinyl, imidazolidin-2-onyl, morpholinyl, morpholin-3-onyl, thiazyl, isothiazyl, isothiazolidine-1,1-dioxidyl, 1,2-thiazinane 1,1-dioxidyl, hexahydrocyclopenta[b]pyrrol-2(1H)-onyl, octahydrocyclopenta[c]pyrrolyl, azetidinyl, hexahydro-1H-indol-2(3H)-onyl, octahydro-1H-isoindolyl, azepanyl, tetrahydrofuranyl, 1,3-dioxolanyl, oxetanyl, cyclopropyl, cyclobutyl, cyclopentyl, cyclohexyl, 4-azepanyl, 1,4-oxazepanyl, tetrahydro-2H-pyranyl, 6,7-dihydro-5H-pyrrolo[1,2-a]imidazolyl, cyclohex-2-enyl, or 1,2,3,4-tetrahydroisoquinolinyl; wherein said alkyl, cycloalkyl or heterocycloalkyl is optionally substituted with one to four R³ and wherein, if the heteroatom on said heterocycloalkyl and heteroaryl is N, said N is optionally substituted with R⁴; or a pharmaceutically acceptable salt of said compound or a tautomer of said compound or said salt.

In a further embodiment, R³ for each occurrence is independently fluoro, chloro, C₁-C₃alkyl, oxo or —OR⁵, wherein said alkyl, is optionally and independently substituted with one to five halogen, —OR⁵, C₃-C₆ cycloalkyl or C₁-C₀₃ alkoxy; or two R³ taken together with the respective carbons to which each are bonded form a 3- to 6-membered cycloalkyl or a 4- to 6-membered heterocycloalkyl, wherein said cycloalkyl or heterocycloalkyl is optionally substituted with one to three halogen, —OR⁵, cyano or C₁-C₆alkyl or C₁-C₆alkoxy, wherein the alkyl or alkoxy is optionally substituted with halogen, —OR⁵; and R⁵ is hydrogen; or a pharmaceutically acceptable salt of said compound or a tautomer of said compound or said salt.

In a further embodiment, R⁴ is independently hydrogen, C₁-C₃alkyl, —C(O)R¹⁰ or —S(O)₂R⁸, wherein the alkyl is optionally substituted with OH, halogen, deuterium, C₁-C₆alkyl, C₁-C₆alkoxy or cyano; R⁸ is C₁-C₃alkyl; R¹⁰ is C₁-C₃alkyl, 3- to 6-membered cycloalkyl, 4- to 6-membered heterocycloalkyl or 5- to 6-membered heteroaryl; wherein said alkyl, cycloalkyl, heterocycloalkyl or heteroaryl is optionally substituted with one to three deuterium, fluoro, OH, C₁-C₃alkoxy, C₁-C₃alkyl (optionally substituted with NR^{11a}R^{11b} or C₁-C₆alkoxy), 3- to 6-membered cycloalkyl, NR^{11a}R^{11b} or cyano; or a pharmaceutically acceptable salt of said compound or a tautomer of said compound or said salt.

In yet another embodiment, R² is selected from

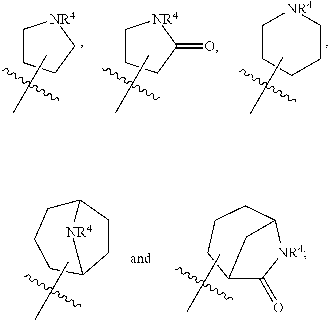

wherein said heterocycloalkyl is optionally substituted with one, two or three R³; or a pharmaceutically acceptable salt of said compound or a tautomer of said compound or said salt.

In one embodiment, R³ is independently selected from fluoro, chloro, hydroxyl and methyl; and R¹ is methyl, ethyl, propyl, isopropyl, butyl, t-butyl, cyclopropyl, cyclobutyl, wherein said R¹ is optionally substituted with one, two or three fluoro; or a pharmaceutically acceptable salt of said compound or a tautomer of said compound or said salt.

In a further embodiment, the invention is selected from the group consisting of:
4-{[(3R)-1-(cyanoacetyl)piperidin-3-yl]oxy}-6-(propan-2-yloxy)quinoline-7-carboxamide;
4-{[(3R)-1-(cyanoacetyl)piperidin-3-yl]oxy}-6-methoxyquinoline-7-carboxamide;
1-{[(3R,4S)-1-(cyanoacetyl)-4-methylpiperidin-3-yl]oxy}-7-(propan-2-yloxy)isoquinoline-6-carboxamide;
4-{[(3R)-1-(cyanoacetyl)piperidin-3-yl]oxy}-6-ethoxyquinoline-7-carboxamide;
1-{[(1S,5R,8R)-7-oxo-6-azabicyclo[3.2.1]oct-3-en-8-yl]oxy}-7-(propan-2-yloxy)isoquinoline-6-carboxamide;
4-chloro-1-{[(3R)-1-(cyanoacetyl)piperidin-3-yl]oxy}-7-(propan-2-yloxy)isoquinoline-6-carboxamide;
5-(piperidin-4-yloxy)-3-(propan-2-yloxy)naphthalene-2-carboxamide;
4-{[(3R,4S)-1-(cyanoacetyl)-4-methylpiperidin-3-yl]oxy}-6-(propan-2-yloxy)quinoline-7-carboxamide;
4-{[(3R)-1-(cyanoacetyl)pyrrolidin-3-yl]oxy}-6-(propan-2-yloxy)quinoline-7-carboxamide;
4-[(1-acetylpiperidin-3-yl)oxy]-6-(propan-2-yloxy)quinoline-7-carboxamide;
4-({(3R)-1-[(2S)-2-amino-3-cyanopropanoyl]piperidin-3-yl}oxy)-6-(propan-2-yloxy)quinoline-7-carboxamide;
4-{[(3R)-1-(cyanoacetyl)piperidin-3-yl]oxy}-6-(difluoromethoxy)quinoline-7-carboxamide;
5-[(3R)-piperidin-3-yloxy]-3-(propan-2-yloxy) naphthalene-2-carboxamide;
4-{[(3R)-1-(cyanoacetyl)piperidin-3-yl]oxy}-2-methyl-6-(propan-2-yloxy)quinoline-7-carboxamide;
4-{[(3S)-1-(cyanoacetyl)pyrrolidin-3-yl]oxy}-6-(propan-2-yloxy)quinoline-7-carboxamide;
4-cyano-1-{[(3R)-1-(cyanoacetyl)piperidin-3-yl]oxy}-7-(propan-2-yloxy)isoquinoline-6-carboxamide;
4-chloro-1-[(3R)-piperidin-3-yloxy]-7-(propan-2-yloxy)isoquinoline-6-carboxamide;
1-[(1-acetylpiperidin-4-yl)oxy]-7-(propan-2-yloxy)isoquinoline-6-carboxamide;
4-{[1-(methylsulfonyl)piperidin-3-yl]oxy}-6-(propan-2-yloxy)quinoline-7-carboxamide;
4-{[(3R)-1-(cyanoacetyl)piperidin-3-yl]oxy}-6-(cyclobutyloxy)quinoline-7-carboxamide;
4-methyl-1-[(3R)-piperidin-3-yloxy]-7-(propan-2-yloxy)isoquinoline-6-carboxamide;
4-({(3R)-1-[(2R)-2-amino-3-cyanopropanoyl]piperidin-3-yl}oxy)-6-(propan-2-yloxy)quinoline-7-carboxamide;
4-{[(3R)-1-(3-cyanopropanoyl)piperidin-3-yl]oxy}-6-(propan-2-yloxy)quinoline-7-carboxamide;
4-{[(3R)-1-(hydroxyacetyl)piperidin-3-yl]oxy}-2-methyl-6-(propan-2-yloxy)quinoline-7-carboxamide;
4-{[(3S,4S)-3-fluoropiperidin-4-yl]oxy}-6-(propan-2-yloxy)quinoline-7-carboxamide;
1-(piperidin-4-yloxy)-7-(propan-2-yloxy)isoquinoline-6-carboxamide;
4-{[(3R,4S)-3-hydroxypiperidin-4-yl]oxy}-6-(propan-2-yloxy)quinoline-7-carboxamide;
1-{[1-(cyanoacetyl)piperidin-4-yl]oxy}-7-(propan-2-yloxy)isoquinoline-6-carboxamide;
6-(difluoromethoxy)-4-[(3R)-piperidin-3-yloxy]quinoline-7-carboxamide;
1-{[1-(hydroxyacetyl)piperidin-4-yl]oxy}-7-(propan-2-yloxy)isoquinoline-6-carboxamide;
4-bromo-1-[(3R)-piperidin-3-yloxy]-7-(propan-2-yloxy)isoquinoline-6-carboxamide;
4-{[(3R,4S)-4-hydroxypiperidin-3-yl]oxy}-6-(propan-2-yloxy)quinoline-7-carboxamide;
1-{[1-(D-alanyl) piperidin-4-yl]oxy}-7-(propan-2-yloxy)isoquinoline-6-carboxamide;
1-{[1-(2-hydroxypropyl)piperidin-4-yl]oxy}-7-(propan-2-yloxy) isoquinoline-6-carboxamide;
1-(piperidin-3-yloxy)-7-(propan-2-yloxy)isoquinoline-6-carboxamide;
4-{[(3R,6S)-6-methylpiperidin-3-yl]oxy}-6-(propan-2-yloxy)quinoline-7-carboxamide;
4-{[(3R)-1-(2-cyanopropanoyl)piperidin-3-yl]oxy}-6-(propan-2-yloxy)quinoline-7-carboxamide;
4-[(2-oxopyrrolidin-3-yl)oxy]-6-(propan-2-yloxy)quinoline-7-carboxamide;
4-(piperidin-4-yloxy)-6-(propan-2-yloxy)quinoline-7-carboxamide;
6-(propan-2-yloxy)-4-[(3S)-pyrrolidin-3-yloxy]quinoline-7-carboxamide;
6-(propan-2-yloxy)-4-(pyrrolidin-3-yloxy)quinoline-7-carboxamide;
4-[(3R)-piperidin-3-yloxy]-6-(propan-2-yloxy)quinoline-7-carboxamide;
6-methoxy-4-[(3R)-piperidin-3-yloxy]quinoline-7-carboxamide;
6-(cyclobutyloxy)-4-[(3R)-piperidin-3-yloxy]quinoline-7-carboxamide;
1-[(3R)-piperidin-3-yloxy]-7-(propan-2-yloxy)isoquinoline-6-carboxamide;
4-{[(3R)-1-(hydroxyacetyl)piperidin-3-yl]oxy}-6-(propan-2-yloxy)quinoline-7-carboxamide;
4-{[(3R)-1-glycylpiperidin-3-yl]oxy}-6-(propan-2-yloxy)quinoline-7-carboxamide;
1-{[(3R)-1-(cyanoacetyl)piperidin-3-yl]oxy}-7-(propan-2-yloxy)isoquinoline-6-carboxamide;
1-{[(3S)-1-(cyanoacetyl)pyrrolidin-3-yl]oxy}-7-(propan-2-yloxy)isoquinoline-6-carboxamide;
1-{[(3R)-1-(cyanoacetyl)pyrrolidin-3-yl]oxy}-7-(propan-2-yloxy)isoquinoline-6-carboxamide;
1-{[(3R,4S)-1-(cyanoacetyl)-3-fluoropiperidin-4-yl]oxy}-7-(propan-2-yloxy)isoquinoline-6-carboxamide;
4-{[(2R,3S)-1-(cyanoacetyl)-2-methylpyrrolidin-3-yl]oxy}-6-(propan-2-yloxy)quinoline-7-carboxamide;
4-{[(3R,4S)-1-(cyanoacetyl)-3-fluoropiperidin-4-yl]oxy}-6-(propan-2-yloxy)quinoline-7-carboxamide;
1-{[(3R)-3-fluoropiperidin-4-yl]oxy}-7-(propan-2-yloxy)isoquinoline-6-carboxamide;
1-[(2-oxopyrrolidin-3-yl)oxy]-7-(propan-2-yloxy) isoquinoline-6-carboxamide;
4-{[(3R)-1-(difluoroacetyl)piperidin-3-yl]oxy}-6-(propan-2-yloxy)quinoline-7-carboxamide;
4-{[(3R)-1-(L-alanyl)piperidin-3-yl]oxy}-6-(propan-2-yloxy)quinoline-7-carboxamide;
4-{[(3R)-1-(methoxyacetyl)piperidin-3-yl]oxy}-6-(propan-2-yloxy)quinoline-7-carboxamide;
6-(propan-2-yloxy)-4-{[(3R)-1-(trifluoroacetyl)piperidin-3-yl]oxy}quinoline-7-carboxamide;
4-{[(3R)-1-propanoylpiperidin-3-yl]oxy}-6-(propan-2-yloxy)quinoline-7-carboxamide;
4-{[(3R)-1-(1,3-oxazol-5-ylcarbonyl)piperidin-3-yl]oxy}-6-(propan-2-yloxy)quinoline-7-carboxamide;
4-{[(3R)-1-(2-methylseryl)piperidin-3-yl]oxy}-6-(propan-2-yloxy)quinoline-7-carboxamide;
4-({(3R)-1-[(2S)-2-hydroxypropanoyl]piperidin-3-yl}oxy)-6-(propan-2-yloxy)quinoline-7-carboxamide;
4-{[(3R)-1-(1H-imidazol-4-ylcarbonyl) piperidin-3-yl]oxy}-6-(propan-2-yloxy)quinoline-7-carboxamide;
4-{[(3R)-1-{[1-(aminomethyl)cyclopropyl]carbonyl}piperidin-3-yl]oxy}-6-(propan-2-yloxy)quinoline-7-carboxamide;
1-{[(3R,4S)-4-methylpiperidin-3-yl]oxy}-7-(propan-2-yloxy)isoquinoline-6-carboxamide;
4-[(1-methylpiperidin-3-yl)oxy]-6-(propan-2-yloxy)quinoline-7-carboxamide;
6-[(2S)-butan-2-yloxy]-4-[(3R)-piperidin-3-yloxy]quinoline-7-carboxamide;
6-(propan-2-yloxy)-4-[(3R)-pyrrolidin-3-yloxy]quinoline-7-carboxamide;
1-[(3-endo)-8-azabicyclo[3.2.1]oct-3-yloxy]-7-(propan-2-yloxy)isoquinoline-6-carboxamide;
7-(propan-2-yloxy)-1-[(3S)-pyrrolidin-3-yloxy]isoquinoline-6-carboxamide;
6-ethoxy-4-[(3R)-piperidin-3-yloxy]quinoline-7-carboxamide;
4-cyano-1-[(3R)-piperidin-3-yloxy]-7-(propan-2-yloxy)isoquinoline-6-carboxamide;
4-{[(3R,4S)-4-methylpiperidin-3-yl]oxy}-6-(propan-2-yloxy)quinoline-7-carboxamide;
1-[(3S)-piperidin-3-yloxy]-7-(propan-2-yloxy)isoquinoline-6-carboxamide;
6-(2-methylpropoxy)-4-[(3R)-piperidin-3-yloxy]quinoline-7-carboxamide;
4-{[(3R,4S)-4-methylpiperidin-3-yl]oxy}-6-(propan-2-yloxy)quinoline-7-carboxamide; and
2-methyl-4-[(3R)-piperidin-3-yloxy]-6-(propan-2-yloxy)quinoline-7-carboxamide; or pharmaceutically acceptable salts thereof or tautomers of said compounds or salt.

In another embodiment, the invention is directed to the intermediate compounds described in the Synthetic Schemes and/or Preparations; or a pharmaceutically acceptable salt of said compound or a tautomer of said compound or said salt

In another embodiment, the invention is directed to a synthetic process and preparation of the intermediate compounds described herein, as detailed in the Schemes and the preparation section described herein. In another aspect, the invention is directed to a synthetic process and preparation of the compounds of Tables 1 or 3, as detailed in the Schemes and the preparation section described herein.

### IRAK4 Indications

The compounds of the invention are also useful in treating and/or preventing a disease or condition mediated by or otherwise associated with an IRAK enzyme; the method comprising administering to a subject in need thereof an effective amount of a compound of the invention.

The disease may be, but not limited to, one of the following classes: auto-immune diseases, inflammatory diseases, allergic diseases, metabolic diseases, infection-based diseases, trauma or tissue-injury based diseases, fibrotic diseases, genetic diseases, diseases driven by over-activity of IL1 pathways, cardiovascular diseases, vascular diseases, heart diseases, neurological diseases, neurodegenerative diseases, respiratory diseases, pulmonary diseases, airways diseases, renal diseases, skin and/or dermatological diseases, liver diseases, gastrointestinal diseases, oral diseases, pain and sensory diseases, hematopoietic diseases, joint diseases, muscle diseases, bone diseases, and ophthalmic and/or ocular diseases.

Specific autoimmune diseases include, but are not limited to: rheumatoid arthritis, osteoarthritis, psoriasis, allergic dermatitis, systemic lupus erythematosus (and resulting complications), Sjögren's syndrome, multiple sclerosis, asthma, glomerular nephritis, irritable bowel syndrome, inflammatory bowel disease, Crohn's disease, ankylosing spondylitis, Behget's disease, lupus nephritis, scleroderma, systemic scleroderma, type 1 or juvenile onset diabetes, alopecia universalis, acute disseminated encephalomyelitis, Addison's disease, antiphospholipid antibody syndrome, atrophic gastritis of pernicious anemia, autoimmune alopecia, autoimmune hemolytic anemia, autoimmune hepatitis, autoimmune encephalomyelitis, autoimmune thrombocytopenia, Bullous pemphigoid, Chagas disease, Celiac disease, chronic hepatitis, Cogan's syndrome, dermatomyositis, endometriosis, Goodpasture's syndrome, Graves' disease, Guillain-Barre syndrome, Hashimoto's disease (or Hashimoto's thyroiditis), hemolytic anemia, hidradentitis suppurativa, idiopathic thrombocytopenia purpura, interstitial cystitis, membranous glomerulopathy, morphea, mystenia gravis, narcolepsy, pemphigus, pernicous anemia, polyarteritis nodosa, polymyositis, primary biliary cirrhosis, Reiter's syndrome, schizophrenia, symphathetic opthalmia, systemic sclerosis, temporal arteritis, thyroiditis, vasculitis, vitiglio, vulvodynia, Wegner's granulomatosis, palmoplantar keratoderma, systemic-onset Juvenile Idiopathic Arthritis (SJIA), or an indication listed in a separate category herein.

Specific inflammatory diseases include, but are not limited to: chronic obstructive pulmonary diseases, airway hyper-responsiveness, cystic fibrosis, acute respiratory distress syndrome, sinusitis, rhinitis, gingivitis, atherosclerosis, chronic prostatitis, glomerular nephritis, ulcerative colitis, uveitis, periodontal disease, or an indication listed in a separate category herein.

Specific pain conditions include, but are not limited to: inflammatory pain, surgical pain, visceral pain, dental pain, premenstrual pain, central pain, pain due to burns, migraine or cluster headaches, nerve injury, interstitial cystitis, cancer pain, viral, parasitic or bacterial infection, post-traumatic injury, pain associated with irritable bowel syndrome, gout, pain associated with any of the other indications listed within this specification, or an indication listed in a separate category herein.

Specific respiratory, airway and pulmonary conditions include, but are not limited to: asthma (which may encompass chronic, late, bronchial, allergic, intrinsic, extrinsic or dust), chronic obstructive pulmonary disease, idiopathic pulmonary fibrosis, pulmonary arterial hypertension, cystic fibrosis, interstitial lung disease, acute lung injury, sarcoidosis, allergic rhinitis, chronic cough, bronchitis, recurrent airway obstruction, emphysema, or bronchospasm, or an indication listed in a separate disease category herein.

Specific gastrointestinal (GI) disorders include, but are not limited to: Irritable Bowel Syndrome (IBS), Inflammatory Bowel Disease (IBD), biliary colic and other biliary disorders, renal colic, diarrhea-dominant IBS, pain associated with GI distension, ulcerative colitis, Crohn's Disease, irritable bowel syndrome, Celiac disease, proctitis, eosinophilic gastroenteritis, mastocytosis, or an indication listed in a separate disease category herein.

Specific allergic diseases include, but are not limited to: anaphylaxis, allergic rhinitis, allergic dermatitis, allergic urticaria, angioedema, allergic asthma, allergic reactions to: food, drugs, insect bites, pollen; or an indication listed in a separate disease category herein.

Specific infection-based diseases include, but are not limited to: sepsis, septic shock, viral diseases, malaria, Lyme disease, ocular infections, conjunctivitis, Whipple Disease, or an indication listed in a separate disease category herein.

Specific trauma and tissue injury-based conditions include, but are not limited to: Renal glomerular damage, reperfusion injury (for example to heart, kidney, lung), spinal cord injury, tissue scarring, tissue adhesion, tissue repair, transplant rejection (for examples to heart, lung, bone marrow, cartilage, cornea, kidney, limb, liver, muscle, myoblast, pancreas, pancreatic islet, skin, nerve, small intestine, trachea), hypersensitivities, or an indication listed in a separate disease category herein.

Specific fibrotic diseases include, but are not limited to: Idiopathic pulmonary fibrosis, liver fibrosis, renal fibrosis, or an indication listed in a separate disease category herein.

Specific diseases considered to be driven by over-activity of IL1 pathways include, but are not limited to: Cryopyrin-associated periodic syndromes, myositis, and indications included in the following review article: C. A. Dinarello, A. Simon and J. W. M. van der Meer, Treating inflammation by blocking interleukin-1 in a broad spectrum of diseases, Nat Rev Drug Discov, 2012, 11(8), 633-652, http://dx.doi.org/10.1038/nrd3800 and supplementary information contained therein, or an indication listed in a separate disease category herein.

Specific ophthalmic/ocular diseases include, but are not limited to: uveitis, age-related macular degeneration, diabetic macular edema, keratoconjuctivitis, uveitis associated with Behget's disease, vernal conjunctivitis, ketatitis, lens-induced uveitis, herpetic keratitis, conical keratitis, corneal epithelial dystrophy, ocular pemphigus, Mooren's ulcer, Scleritis, Graves' ophthalmopathy, Vogt-Koyanagi-Harada syndrome, keratoconjunctivitis sicca, phlyctenule, iridocyclitis, sympathetic ophthalmia, allergic conjunctivitis, ocular neovascularization, dry eye syndrome, or an indication listed in a separate disease category herein.

Specific joint, muscle and bone disorders include, but are not limited to: osteoarthritis, osteoporosis, rheumatoid arthritis, juvenile arthritis, psoriatic arthritis, erosive osteoarthritis of the hand, arthrofibrosis/traumatic knee injury, anterior cruciate knee ligament tear, relapsing polychondritis, recurrent multifocal osteomyelitis, Majeed Syndrome, ankylosing spondylitis, gout of the lumbar spine, antisynthetase syndrome, idiopathic inflammatory myopathies, articular chondrocalcinosis, systemic-onset Juvenile Idiopathic Arthritis (SJIA), gout and pyrophosphate crystal arthritis, or an indication listed in a separate disease category herein.

Specific skin/dermatological diseases include, but are not limited to: psoriasis, atopic dermatitis, cutaneous lupus, acne, dermatomyositis, eczema, pruritus, scleroderma, Sweet Syndrome/neutrophilic dermatosis, neutrophilic panniculitis, acrodermatitis (form of pustular psoriasis), or an indication listed in a separate disease category herein.

Specific renal diseases include, but are not limited to: acute kidney injury (AKI) (sepsis-AKI, coronary artery bypass graft-AKI, cardiac surgery-AKI, non-cardiac surgery-AKI, transplant surgery-AKI cisplatin-AKI, contrast/imaging agent induced-AKI), glomerulonephritis, IgA nephropathy, crescentic GN, lupus nephritis, HIV associated nephropathy, membraneous nephropathy, C3 glomerulopathy, Dense deposit disease, ANCA vasculitis, diabetic nephropathy, hemolytic-uremic syndrome, atypical Hemolytic-uremic syndrome, nephrotic syndrome, nephritic syndrome, hypertensive nephrosclerosis, ApoL1 nephropathy, focal segmental glomerulosclerosis, Alport syndrome, Fanconi syndrome, crystal nephropathy, nephrolithiasis, nephrotic syndrome, renal transplant rejection, amyloidosis, glomerulonephritis in SJIA, or an indication listed in a separate disease category herein.

Specific genetic diseases include, but are not limited to: Familial Mediterranean fever (FMF), CAPS (FCAS, Muckle-Wells Syndrome, NOMID/CINCA), male hypoinfertility in CAPS, NLRP12 Autoinflammatory Syndrome, or an indication listed in a separate disease category herein.

Specific hematopoietic diseases include, but are not limited to: hemolytic anemia, or an indication listed in a separate disease category herein.

Specific liver diseases include, but are not limited to: liver fibrosis, liver cirrhosis, nonalcoholic steatohepatitis (NASH), or an indication listed in a separate disease category herein.

Specific oral diseases include, but are not limited to: gingivitis, periodontal disease or an indication listed in a separate disease category herein.

Specific metabolic diseases include, but are not limited to: Type 2 diabetes (and resulting complications), gout and hyperuricemia, metabolic syndrome, insulin resistance, obesity, or an indication listed in a separate disease category herein.

Compounds of the current invention are also useful in the treatment of a proliferative disease selected from a benign or malignant tumor, solid tumor, carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, nonsmall-cell lung carcinoma, lymphomas, Hodgkins and Non-Hodgkins, mammary carcinoma, follicular carcinoma, undifferentiated carcinoma, papillary carcinoma, seminoma, melanoma, smoldering of indolent multiple myeloma, or hematological malignancies (including leukemia, diffuse large B-cell lymphoma (DLBCL), ABC DLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, Waldenstrom's macroglobulinemia (WM), splenic marginal zone lymphoma, multiple myeloma, plasmacytoma, intravascular large B-cell lymphoma), or an indication listed in a separate disease category herein.

Cardiovascular conditions include, but are not limited to coronary heart disease, acute coronary syndrome, ischaemic heart disease, first or recurrent myocardial infarction, secondary myocardial infarction, non-ST segment elevation myocardial infarction, or ST segment elevation myocardial infarction, ischemic sudden death, transient ischemic attack, peripheral occlusive arterial disease, angina, atherosclerosis, hypertension, heart failure (such as congestive heart failure), diastolic dysfunction (such as left ventricular diastolic dysfunction, diastolic heart failure, and impaired diastolic filling), systolic dysfunction (such as systolic heart failure with reduced ejection fraction), vasculitis, ANCA vasculitis, post-myocardial infarction cardiac remodeling atrial fibrillation, arrhythmia (ventricular), ischemia, hypertrophic cardiomyopathy, sudden cardiac death, myocardial and vascular fibrosis, impaired arterial compliance, myocardial necrotic lesions, vascular damage, left ventricular hypertrophy, decreased ejection fraction, cardiac lesions, vascular wall hypertrophy, endothelial thickening, fibrinoid necrosis of coronary arteries, adverse remodeling, stroke, and the like, or an indication listed in a separate disease category herein. Also, included are venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, or (f) other procedures in which blood is exposed to an artificial surface that promotes thrombosis. It is noted that thrombosis includes occlusion (e.g., after a bypass) and reocclusion (e.g., during or after percutaneous transluminal coronary angioplasty).

Cardiovascular complications of type 2 diabetes are associated with inflammation, accordingly, the compounds of the present invention may be used to treat diabetes and diabetic complications such as macrovascular disease, hyperglycemia, metabolic syndrome, impaired glucose tolerance, hyperuricemia, glucosuria, cataracts, diabetic neuropathy, diabetic nephropathy, diabetic retinopathy, obesity, dyslipidemia, hypertension, hyperinsulinemia, and insulin resistance syndrome, or an indication listed in a separate disease category herein.

Linkage of innate immunity and inflammation to disease has been demonstrated in neuroinflammatory and neurodegenerative conditions. Therefore, the compounds of the present invention are particularly indicated for use in the treatment of neuroinflammatory and neurodegenerative conditions (i.e., disorders or diseases) in mammals including humans such as multiple sclerosis, migraine; epilepsy; Alzheimer's disease; Parkinson's disease; brain injury; stroke; cerebrovascular diseases (including cerebral arteriosclerosis, cerebral amyloid angiopathy, hereditary cerebral hemorrhage, and brain hypoxia-ischemia); cognitive disorders (including amnesia, senile dementia, HIV associated dementia, Alzheimer's associated dementia, Huntington's associated dementia, Lewy body dementia, vascular dementia, drug related dementia, delirium, and mild cognitive impairment); mental deficiency (including Down syndrome and fragile X syndrome); sleep disorders (including hypersomnia, circadian rhythm sleep disorder, insomnia, parasomnia, and sleep deprivation) and psychiatric disorders (such as anxiety (including acute stress disorder, generalized anxiety disorder, social anxiety disorder, panic disorder, post-traumatic stress disorder and obsessive-compulsive disorder); factitious disorder (including acute hallucinatory mania); impulse control disorders (including compulsive gambling and intermittent explosive disorder); mood disorders (including bipolar I disorder, bipolar II disorder, mania, mixed affective state, major depression, chronic depression, seasonal depression, psychotic depression, and postpartum depression); psychomotor disorder; psychotic disorders (including schizophrenia, schizoaffective disorder, schizophreniform, and delusional disorder); drug dependence (including narcotic dependence, alcoholism, amphetamine dependence, cocaine addiction, nicotine dependence, and drug withdrawal syndrome); eating disorders (including anorexia, bulimia, binge eating disorder, hyperphagia, and pagophagia); and pediatric psychiatric disorders (including attention deficit disorder, attention deficit/hyperactive disorder, conduct disorder, and autism), myotrophic lateral sclerosis, chronic fatigue syndrome, or an indication listed in a separate disease category herein.

Typically, a compound of the invention is administered in an amount effective to treat a condition as described herein. The compounds of the invention are administered by any suitable route in the form of a pharmaceutical composition adapted to such a route, and in a dose effective for the treatment intended. Therapeutically effective doses of the compounds required to treat the progress of the medical condition are readily ascertained by one of ordinary skill in the art using preclinical and clinical approaches familiar to the medicinal arts.

The compounds of the invention may be administered orally. Oral administration may involve swallowing, so that the compound enters the gastrointestinal tract, or buccal or sublingual administration may be employed, by which the compound enters the blood stream directly from the mouth.

In another embodiment, the compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

In another embodiment, the compounds of the invention may also be administered topically to the skin or mucosa, that is, dermally or transdermally. In another embodiment, the compounds of the invention can also be administered intranasally or by inhalation. In another embodiment, the compounds of the invention may be administered rectally or vaginally. In another embodiment, the compounds of the invention may also be administered directly to the eye or ear.

The dosage regimen for the compounds and/or compositions containing the compounds is based on a variety of factors, including the type, age, weight, sex and medical condition of the patient; the severity of the condition; the route of administration; and the activity of the particular compound employed. Thus the dosage regimen may vary widely. Dosage levels of the order from about 0.01 mg to about 100 mg per kilogram of body weight per day are useful in the treatment of the above-indicated conditions. In one embodiment, the total daily dose of a compound of the invention (administered in single or divided doses) is typically from about 0.01 to about 100 mg/kg. In another embodiment, the total daily dose of the compound of the invention is from about 0.1 to about 50 mg/kg, and in another embodiment, from about 0.5 to about 30 mg/kg (i.e., mg compound of the invention per kg body weight). In one embodiment, dosing is from 0.01 to 10 mg/kg/day. In another embodiment, dosing is from 0.1 to 1.0 mg/kg/day. Dosage unit compositions may contain such amounts or submultiples thereof to make up the daily dose. In many instances, the administration of the compound will be repeated a plurality of times in a day (typically no greater than 4 times). Multiple doses per day typically may be used to increase the total daily dose, if desired.

For oral administration, the compositions may be provided in the form of tablets containing from about 0.01 mg to about 500 mg of the active ingredient, or in another embodiment, from about 1 mg to about 100 mg of active ingredient. Intravenously, doses may range from about 0.1 to about 10 mg/kg/minute during a constant rate infusion.

Suitable subjects according to the present invention include mammalian subjects. Mammals according to the present invention include, but are not limited to, canine, feline, bovine, caprine, equine, ovine, porcine, rodents, lagomorphs, primates, and the like, and encompass mammals in utero. In one embodiment, humans are suitable subjects. Human subjects may be of either gender and at any stage of development.

In another embodiment, the invention comprises the use of one or more compounds of the invention for the preparation of a medicament for the treatment of the conditions recited herein.

For the treatment of the conditions referred to above, the compound of the invention can be administered as compound per se. Alternatively, pharmaceutically acceptable salts are suitable for medical applications because of their greater aqueous solubility relative to the parent compound.

In another embodiment, the present invention comprises pharmaceutical compositions. Such pharmaceutical compositions comprise a compound of the invention presented with a pharmaceutically acceptable carrier. The carrier can be a solid, a liquid, or both, and may be formulated with the compound as a unit-dose composition, for example, a tablet, which can contain from 0.05% to 95% by weight of the active compounds. A compound of the invention may be coupled with suitable polymers as targetable drug carriers. Other pharmacologically active substances can also be present.

The compounds of the present invention may be administered by any suitable route, preferably in the form of a pharmaceutical composition adapted to such a route, and in a dose effective for the treatment intended. The active compounds and compositions, for example, may be administered orally, rectally, parenterally, or topically.

Oral administration of a solid dose form may be, for example, presented in discrete units, such as hard or soft capsules, pills, cachets, lozenges, or tablets, each containing a predetermined amount of at least one compound of the present invention. In another embodiment, the oral administration may be in a powder or granule form. In another embodiment, the oral dose form is sub-lingual, such as, for example, a lozenge. In such solid dosage forms, the compounds of Formula Ia are ordinarily combined with one or more adjuvants. Such capsules or tablets may contain a controlled-release formulation. In the case of capsules, tablets, and pills, the dosage forms also may comprise buffering agents or may be prepared with enteric coatings.

In another embodiment, oral administration may be in a liquid dose form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as wetting, emulsifying, suspending, flavoring (e.g., sweetening), and/or perfuming agents.

In another embodiment, the present invention comprises a parenteral dose form. “Parenteral administration” includes, for example, subcutaneous injections, intravenous injections, intraperitoneal injections, intramuscular injections, intrasternal injections, and infusion. Injectable preparations (e.g., sterile injectable aqueous or oleaginous suspensions) may be formulated according to the known art using suitable dispersing, wetting agents, and/or suspending agents.

In another embodiment, the present invention comprises a topical dose form. “Topical administration” includes, for example, transdermal administration, such as via transdermal patches or iontophoresis devices, intraocular administration, or intranasal or inhalation administration. Compositions for topical administration also include, for example, topical gels, sprays, ointments, and creams. A topical formulation may include a compound that enhances absorption or penetration of the active ingredient through the skin or other affected areas.

When the compounds of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated; see, for example, J. Pharm. Sci., 88(10), 955-958, by Finnin and Morgan (October 1999).

Formulations suitable for topical administration to the eye include, for example, eye drops wherein the compound of this invention is dissolved or suspended in a suitable carrier. A typical formulation suitable for ocular or aural administration may be in the form of drops of a micronized suspension or solution in isotonic, pH-adjusted, sterile saline. Other formulations suitable for ocular and aural administration include ointments, biodegradable (e.g., absorbable gel sponges, collagen) and non-biodegradable (e.g., silicone) implants, wafers, lenses and particulate or vesicular systems, such as niosomes or liposomes. A polymer such as cross-linked polyacrylic acid, polyvinyl alcohol, hyaluronic acid, a cellulosic polymer, for example, hydroxypropyl methyl cellulose, hydroxyethyl cellulose, or methyl cellulose, or a heteropolysaccharide polymer, for example, gelan gum, may be incorporated together with a preservative, such as benzalkonium chloride. Such formulations may also be delivered by iontophoresis.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the present invention comprises a rectal dose form. Such rectal dose form may be in the form of, for example, a suppository. Cocoa butter is a traditional suppository base, but various alternatives may be used as appropriate.

Other carrier materials and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman et al., Eds., Pharmaceutical Dosage Forms, Marcel Decker, New York, N.Y., 1980; and Kibbe et al., Eds., Handbook of Pharmaceutical Excipients (3^{rd} Ed.), American Pharmaceutical Association, Washington, 1999.

The compounds of the present invention can be used, alone or in combination with other therapeutic agents, in the treatment of various conditions or disease states. The compound(s) of the present invention and other therapeutic agent(s) may be may be administered simultaneously (either in the same dosage form or in separate dosage forms) or sequentially.

Two or more compounds may be administered simultaneously, concurrently or sequentially. Additionally, simultaneous administration may be carried out by mixing the compounds prior to administration or by administering the compounds at the same point in time but at different anatomic sites or using different routes of administration.

The phrases “concurrent administration,” “co-administration,” “simultaneous administration,” and “administered simultaneously” mean that the compounds are administered in combination.

The present invention includes the use of a combination of an IRAK inhibitor compound as provided in the compound of Formula Ia and one or more additional pharmaceutically active agent(s). If a combination of active agents is administered, then they may be administered sequentially or simultaneously, in separate dosage forms or combined in a single dosage form. Accordingly, the present invention also includes pharmaceutical compositions comprising an amount of: (a) a first agent comprising a compound of Formula Ia or a pharmaceutically acceptable salt of the compound; (b) a second pharmaceutically active agent; and (c) a pharmaceutically acceptable carrier, vehicle or diluent.

The compounds of the present invention can be administered alone or in combination with one or more additional therapeutic agents. By “administered in combination” or “combination therapy” it is meant that a compound of the present invention and one or more additional therapeutic agents are administered concurrently to the mammal being treated. When administered in combination each component may be administered at the same time or sequentially in any order at different points in time. Thus, each component may be administered separately but sufficiently closely in time so as to provide the desired therapeutic effect. Thus, the methods of prevention and treatment described herein include use of combination agents.

The combination agents are administered to a mammal, including a human, in a therapeutically effective amount. By “therapeutically effective amount” it is meant an amount of a compound of the present invention that, when administered alone or in combination with an additional therapeutic agent to a mammal, is effective to treat the desired disease/condition e.g., inflammatory condition such as systemic lupus erythematosus. See also, T. Koutsokeras and T. Healy, Systemic lupus erythematosus and lupus nephritis, Nat Rev Drug Discov, 2014, 13(3), 173-174, for therapeutic agents useful treating lupus.

In particular, it is contemplated that the compounds of the invention may be administered with the following therapeutic agents:

Non-steroidal anti-inflammatory drugs (NSAIDs), including but not limited to, non-selective COX1/2 inhibitors such as piroxicam, naproxen, flubiprofen, fenoprofen, ketoprofen, ibuprofen, etodolac (Lodine), mefanamic acid, sulindac, apazone, pyrazolones (such as phenylbutazone), salicylates (such as aspirin); selective COX2 inhibitors such as: celecoxib, rofecoxib, etoricoxib, valdecoxib, meloxicam;

Immunomodulatory and/or anti-inflammatory agents, including but not limited to, methotrexate, leflunomide, ciclesonide chloroquine, hydroxychloroquine, d-penicillamine, auranofin, sulfasalazine, sodium aurothiomalate, cyclosporine, azathioprine, cromolyn, hydroxycarbamide, retinoids, fumarates (such as monomethyl and dimethyl fumarate), glatiramer acetate, mitoxantrone, teriflunomide, suplatast tosilate, mycophenolate mofetil and cyclophosphamide, laquinimod, voclosporin, PUR-118, AMG 357, AMG 811, BCT197;

Antimalarials, including but not limited to, hydroxychloroquine (Plaquenil) and chloroquine (Aralen), cyclophosphamide (Cytoxan), methotrexate (Rheumatrex), azathioprine (Imuran), mesalamine (Asacol) and sulfasalazine (Azulfidine):

Antibiotics, including but not limited to, Flagyl or ciprofloxacin;

Anti-TNFα agents, including but not limited to, infliximab, adalimumab, certolizumab pegol, golimumab and etanercept;

Anti-CD20 agents, including but not limited to, rituximab, ocrelizumab, ofatumumab and PF-05280586;

Antidiarrheals, such as diphenoxylate (Lomotil) and loperamide (Imodium);

Bile acid binding agents, such as cholestyramine, alosetron (Lotronex) and ubiprostone (Amitiza);

Laxatives, such as Milk of Magnesia, polyethylene glycol (MiraLax), Dulcolax, Correctol and Senokot, and anticholinergics or antispasmodics such as dicyclomine (Bentyl);

T lymphocyte activation inhibitors, including but not limited to, abatacept:

Anti-IL1 treatments, including but not limited to, anakinra, rilonacept, canakinumab, gevokizumab, MABpl and MEDI-8968;

Glucocorticoid receptor modulators that may be dosed orally, by inhalation, by injection, topically, rectally, by ocular delivery, including but not limited to, betamethasone, prednisone, hydrocortisone, prednisolone, flunisolide, triamcinoline acetonide, beclomethasone, dipropionate, budesonide, fluticasone propionate, ciclesonide, mometasone furoate, fluocinonide, desoximetasone, methylprednisolone or PF-04171327;

Aminosalicyic acid derivatives, including but not limited to, sulfasalazine and mesalazine;

Anti-α4 integrin agents, including but not limited to, natalizumab;

α1- or a2-adrenergic agonist agents including but not limited to: propylhexidrine, phenylephrine, phenylpropanolamine, pseudoephedrine or naphazoline hydrochloride, oxymetazoline hydrochloride, tetrahydrozoline hydrochloride, xylometazoline hydrochloride or ethylnorepinephrine hydrochloride;

β-adrenergic agonists, including but not limited to, metaproterenol, isoprotenerol, isoprenaline, albuterol, salbutamol, formoterol, salmeterol, terbutaline, orciprenaline, botolterol mesylate, pirbuterol;

Anticholinergic agents, including but not limited to, ipratropium bromide, tiotropium bromide, oxitropium bromide, aclindinium bromide, glycopyrrolate, pirenzipine or telenzepine;

Inhaled long acting beta-agonists, long acting muscarinic antagonists and long acting corticosteroids, including but not limited, to those included in the following reference: Y. Mushtaq, The COPD pipeline, Nat Rev Drug Discov, 2014, 13(4), 253-254. http://dx.doi.org/10.1038/nrd425;

Leukotriene pathway modulators, including but not limited to, 5-LO inhibitors (such as zileuton), FLAP antagonists (such as veliflapon, fiboflapon), LTD4 antagonists (such as montelukast, zafirlukast or pranlukast;

H1 receptor antagonists, including but not limited to, cetirizine, loratidine, desloratidine, fexofenadine, astemizole, azelastine or chlorpheniramine;

PDE4 inhibitors, including but not limited to, apremilast, roflumilast or AN2728;

Vitamin D receptor modulators, including but not limited to, paricalcitol;

Nrf2 pathway activators, including but not limited to, fumarates, sulfurophane and bardoxolone methyl;

Modulators of the RAR-related orphan receptor (ROR) family, in particular RORg;

Modulator and/or antagonists of the chemokine receptors, including but not limited to, CCR2 antagonists (such as CCX140, BMS-741672, PF-4634817, CCX-872, NOX-E36), CCR2/5 antagonists (such as PF-4634817), CCR9 (such as vercirnon, CCX507), CCR1 modulators, CCR4 modulators, CCR5 modulators, CCR6 modulators, CXCR6 modulators, CXCR7 modulators) and CXCR2 modulators (such as danirixin, AZD5069);

Prostaglandins, including but not limited to, prostacyclin;

PDE5 inhibitors, including but not limited to, sildenafil, PF-489791, vardenafil and tadalafil;

Endothelin receptor antagonists, including but not limited to, bosentan, ambrisentan, sparsentan, atrasentan, zibotentan and macitentan;

Soluble guanylate cyclase activators, including but not limited to, riociguat;

Interferons, including but not limited to, interferon beta-la interferon beta-1b;

Sphingosine 1-phosphate receptor modulators, including but not limited to, fingolimod and ponesimod;

Inhibitors of the complement pathway, including but not limited to, C5aR antagonists (such as CCX168, PMX-53, NN8210), C5 inhibitors (such as eculizumab), inhibitors of complement factors B and D, inhibitors of MASP2 (such as OMS-721) and ARC-1905;

Inhibitors of Janus kinases (one of more of JAK1, JAK2, JAK3, TYK2), including but not limited to, decernotinib, cerdulatinib, JTE-052, ruxolitinib, tofacitnib, Baricitinib, Peficitinib, GLPG-0634, INCB-47986, INCB-039110, PF-04965842, XL-019, ABT-494, R-348, GSK-2586184, AC-410, BMS-911543 and PF-06263276;

Inhibitors of other anti-inflammatory or immunomodulatory kinases, including but not limited to, spleen tyrosine kinase (SYK) inhibitors, p38 MAP kinase inhibitors (such as PF-3715455, PH-797804, AZD-7624, AKP-001, UR-13870, FX-005, semapimod, pexmetinib, ARRY-797, RV-568, dilmapimod, ralimetinib), PI3K inhibitors (such as GSK-2126458, pilaralisib, GSK-2269557), PI3Kg and/or PI3Kd inhibitors (such as CAL-101/GS-1101, duvelisib), JNK inhibitors, ERK1 and/or 2 inhibitors, IKKb inhibitors, BTK inhibitors, ITK inhibitors, ASK1 inhibitors (such as GS-4997), PKC inhibitors (such as sotrastaurin), TrkA antagonists (such as CT-327), MEK1 inhibitors (such as E6201);

Antioxidants, including but not limited to, myeloperoxidase inhibitors (such as AZD-3241), NOX4 and other NOX enzymes (such as GKT-137831) and N-acetyl cysteine;

Inhibitors of IL5, including but not limited to, mepolizumab, reslizumab and benralizumab;

Inhibitors of IL4, including but not limited to, pascolizumab, altrakincept and pitrakinra;

Inhibitors of IL13, including but not limited to, tralokinumab, anrukinzumab and lebrikizumab;

Anti-IL6 agents, including but not limited to, tocilizumab, olokizumab, siltuximab, PF-4236921 and sirukumab;

Inhibitors/Antagonists of IL17/IL17R, including but not limited to, secukinumab, RG-7624, brodalumab and ixekizumab;

Antagonists of IL12 and/or IL23, including but not limited to, tildrakizumab, guselkumab, MEDI2070 and AMG 139;

Inhibitors of IL33, including but not limited to, AMG 282;

Inhibitors of IL9, including but not limited to, MEDI-528;

Inhibitors of GM-CSF, including but not limited to, MT203;

Anti CD4 agents, including but not limited to, tregalizumab and rigerimod;

CRTH2 antagonists, including but not limited to, AZD-1981;

Inhibitors of B lymphocyte stimulator (BLYS; also known as BAFF), a protein that is often increased in patients with SLE, including but not limited to, belimumab, tabalumab, blisibimod, and atacicept;

CD22-specific monoclonal antibodies, including but not limited to, epratuzumab;

Inhibitors of interferon-α, including but not limited to, sifalimumab and rontalizumab;

Inhibitor of type I interferon receptors, including but not limited to, MEDI-546;

FcγRIIB agonists, including but not limited to, SM-101;

Modified and/or recombinant versions of Heat Shock Protein 10 (Hsp10, also known as Chaperonin 10 or EPF), including but not limited to, INV-103;

Inhibitors of the TNF superfamily receptor 12A (TWEAK receptor), including but not limited to, BIIB-023, enavatuzumab, and RG-7212;

Inhibitors of xanthine oxidase, including but not limited to, allopurinol, benzbromarone, febuxostat, topiroxostat, tisopurine and inositols;

Inhibitors of URAT1 (also known as SLC22A12), including but not limited to, lesinurad, RDEA 3170, UR1102 and levotofispam;

Additional treatments for gout and/or lowering of uric acid levels, including but not limited to, colchicines, pegloticase, benziodarone, isobrominidione, BCX4208 and arhalofenate;

Inhibitors of toll-like receptors (TLRs), including but not limited to, one or more of TLR7, TLR8, TLR9 (such as IMO-8400, IMO-3100, DV-1179), TLR2 and/or TLR 4 (such as VB-201, OPN-305);

Agonists of TLRs, including but not limited to, TLR7 (such as GSK2245035, AZD8848), TLR9 (such as AZD1419);

Activators SIRT1, including but not limited to, SRT2104;

A3 receptor agonists, including but not limited to, CF101;

Other agents of use of the treatment of psoriasis, including but not limited to, IDP-118, LAS41004, LEO 80185, LEO 90100, PH-10, WBI-1001, CNT01959, BT-061, cimzia, ustekinumab, MK-3222/SCH 900222, ACT-128800, AEB071, alitretinoin, ASP015K, Apo805K1, BMS-582949, FP187, hectoral (doxercalciferol), LEO 22811, Ly3009104 (INCB28050), calcipotriene foam (STF 115469), tofacitinib (CP-690,550), M518101 and CycloPsorb™;

Antifibrotic agents, including but not limited to: pirfenidone, inhibitors of LOXL2 (such as Simtuzumab), FT-011, modulators of epiregulin and/or TGFα (such as LY-3016859), modulators of TGFβ (such as LY-2382770, fresolimumab);

Prolyl hydroxylase inhibitors, including but not limited to, GSK1278863, FG-2216, ASP-1517/FG-4592, AKB-6548, JTZ-951, BAY-85-3934 and DS-1093;

Inhibitors of granulocyte macrophage colony-stimulating factor, including but not limited to, GSK3196165 (MOR103), PD-0360324 and mavrilimumab;

Inhibitors of MAdCAM and/or α4β7 integrin, including but not limited to, PF-00547659 and MEDI7183 (abrilumab);

Inhibitors of connective tissue growth factor (CTGF), including but not limited to, PF-06473871; Inhibitors of cathepsin C, including but not limited to, GSK2793660;

Inhibitors of soluble epoxide hydrolase, including but not limited to, GSK2269557;

Inhibitors of the TNFR1 associated death domain protein, including but not limited to, GSK2862277;

Anti-CD19 agents, including but not limited to, MEDI-551 and AMG 729;

Anti-B7RP1 agents/inhibitors of ICOS ligand, including but not limited to, MEDI5872 and AMG-557;

Inhibitors of thymic stromal lymphoprotein, including but not limited to, AMG157;

Inhibitors of IL2, including but not limited to, daclizumab;

Inhibitors of Leucine rich repeat neuronal protein 6A, including but not limited to, Anti-Lingo (Biogen);

Inhibitors of integrins, including but not limited to, alpha-V/beta-6 (STX-100) and alpha-V/beta-3 (VPI-2690B);

Anti-CD40L agents, including but not limited to, CDP-7657;

Modulators of the dopamine D3 receptor, including but not limited to, ABT-614;

Inhibitors and/or modulators of galectin-3, including but not limited to, GCS-100 and GR-MD-02;

Agents for treating diabetic nephropathy, including but not limited to, DA-9801 and ASP-8232;

Agents for treating acute kidney injury, including but not limited to, THR-184, TRC-160334, NX-001, EA-230, ABT-719, CMX-2043, BB-3 and MTP-131;

Modulators of inflammasomes, including but not limited to, inhibitors of NLRP3;

Modulators of bromodomains, including but not limited to, BRD4;

Modulators of GPR43; and

Inhibitors of TRP channels, including but not limited to, TRPA1, TRPC3, TRPC5, TRPC6 and TRPC6.

Additional therapeutic agents include anti-coagulant or coagulation inhibitory agents, anti-platelet or platelet inhibitory agents, thrombin inhibitors, thrombolytic or fibrinolytic agents, anti-arrhythmic agents, anti-hypertensive agents, calcium channel blockers (L-type and T-type), cardiac glycosides, diuretics, mineralocorticoid receptor antagonists, NO donating agents such as organonitrates, NO promoting agents such as phosphodiesterase inhibitors, cholesterol/lipid lowering agents and lipid profile therapies, anti-diabetic agents, anti-depressants, anti-inflammatory agents (steroidal and non-steroidal), anti-osteoporosis agents, hormone replacement therapies, oral contraceptives, anti-obesity agents, anti-anxiety agents, anti-proliferative agents, anti-tumor agents, anti-ulcer and gastroesophageal reflux disease agents, growth hormone and/or growth hormone secretagogues, thyroid mimetics (including thyroid hormone receptor antagonist), anti-infective agents, anti-viral agents, anti-bacterial agents, and anti-fungal agents.

Agents used in an ICU setting are included, for example, dobutamine, dopamine, epinephrine, nitroglycerin, nitroprusside, etc.

Combination agents useful for treating vasculitis are included, for example, azathioprine, cyclophosphamide, mycophenolate, mofetil, rituximab, etc.

In another embodiment, the present invention provides a combination wherein the second agent is at least one agent selected from a factor Xa inhibitor, an anti-coagulant agent, an anti-platelet agent, a thrombin inhibiting agent, a thrombolytic agent, and a fibrinolytic agent. Exemplary factor Xa inhibitors include apixaban and rivaroxaban. Examples of suitable anti-coagulants for use in combination with the compounds of the present invention include heparins (e.g., unfractioned and low molecular weight heparins such as enoxaparin and dalteparin).

In another embodiment the second agent is at least one agent selected from warfarin, unfractionated heparin, low molecular weight heparin, synthetic pentasaccharide, hirudin, argatrobanas, aspirin, ibuprofen, naproxen, sulindac, indomethacin, mefenamate, droxicam, diclofenac, sulfinpyrazone, piroxicam, ticlopidine, clopidogrel, tirofiban, eptifibatide, abciximab, melagatran, disulfatohirudin, tissue plasminogen activator, modified tissue plasminogen activator, anistreplase, urokinase, and streptokinase.

In another embodiment, the agent is at least one anti-platelet agent. Especially preferred anti-platelet agents are aspirin and clopidogrel. The term anti-platelet agents (or platelet inhibitory agents), as used herein, denotes agents that inhibit platelet function, for example by inhibiting the aggregation, adhesion or granular secretion of platelets. Agents include, but are not limited to, the various known non-steroidal anti-inflammatory drugs (NSAIDS) such as aspirin, ibuprofen, naproxen, sulindac, indomethacin, mefenamate, droxicam, diclofenac, sulfinpyrazone, piroxicam, and pharmaceutically acceptable salts or prodrugs thereof. Of the NSAIDS, aspirin (acetylsalicyclic acid or ASA) and COX-2 inhibitors such as celecoxib or piroxicam are preferred. Other suitable platelet inhibitory agents include lib/Illa antagonists (e.g., tirofiban, eptifibatide, and abciximab), thromboxane-A2-receptor antagonists (e.g., ifetroban), thromboxane-A2-synthetase inhibitors, PDE-III inhibitors (e.g., Pletal, dipyridamole), and pharmaceutically acceptable salts or prodrugs thereof.

The term anti-platelet agents (or platelet inhibitory agents), as used herein, is also intended to include ADP (adenosine diphosphate) receptor antagonists, preferably antagonists of the purinergic receptors P₂Y₁ and P₂Y₁₂, with P₂Y₁₂ being even more preferred. Preferred P₂Y₁₂ receptor antagonists include ticagrelor, prasugrel, ticlopidine and clopidogrel, including pharmaceutically acceptable salts or prodrugs thereof. Clopidogrel is an even more preferred agent. Ticlopidine and clopidogrel are also preferred compounds since they are known to be gentle on the gastro-intestinal tract in use.

The term thrombin inhibitors (or anti-thrombin agents), as used herein, denotes inhibitors of the serine protease thrombin. By inhibiting thrombin, various thrombin-mediated processes, such as thrombin-mediated platelet activation (that is, for example, the aggregation of platelets, and/or the granular secretion of plasminogen activator inhibitor-1 and/or serotonin) and/or fibrin formation are disrupted. A number of thrombin inhibitors are known to one of skill in the art and these inhibitors are contemplated to be used in combination with the present compounds. Such inhibitors include, but are not limited to, boroarginine derivatives, boropeptides, heparins, hirudin, argatroban, and melagatran, including pharmaceutically acceptable salts and prodrugs thereof. Boroarginine derivatives and boropeptides include N-acetyl and peptide derivatives of boronic acid, such as C-terminal alpha-aminoboronic acid derivatives of lysine, ornithine, arginine, homoarginine and corresponding isothiouronium analogs thereof. The term hirudin, as used herein, includes suitable derivatives or analogs of hirudin, referred to herein as hirulogs, such as disulfatohirudin. The term thrombolytics or fibrinolytic agents (or thrombolytics or fibrinolytics), as used herein, denote agents that lyse blood clots (thrombi). Such agents include tissue plasminogen activator (natural or recombinant) and modified forms thereof, anistreplase, urokinase, streptokinase, tenecteplase (TNK), lanoteplase (nPA), factor VIIa inhibitors, PAI-1 inhibitors (i.e., inactivators of tissue plasminogen activator inhibitors), alpha2-antiplasmin inhibitors, and anisoylated plasminogen streptokinase activator complex, including pharmaceutically acceptable salts or prodrugs thereof. The term anistreplase, as used herein, refers to anisoylated plasminogen streptokinase activator complex, as described, for example, in EP 028,489, the disclosure of which is hereby incorporated herein by reference herein. The term urokinase, as used herein, is intended to denote both dual and single chain urokinase, the latter also being referred to herein as prourokinase. Examples of suitable anti-arrythmic agents include: Class I agents (such as propafenone); Class II agents (such as metoprolol, atenolol, carvadiol and propranolol); Class III agents (such as sotalol, dofetilide, amiodarone, azimilide and ibutilide); Class IV agents (such as ditiazem and verapamil); K⁺ channel openers such as I_{Ach} inhibitors, and I_{Kur} inhibitors (e.g., compounds such as those disclosed in WO01/40231).

The compounds of the present invention may be used in combination with antihypertensive agents and such antihypertensive activity is readily determined by those skilled in the art according to standard assays (e.g., blood pressure measurements). Examples of suitable anti-hypertensive agents include: alpha adrenergic blockers; beta adrenergic blockers; calcium channel blockers (e.g., diltiazem, verapamil, nifedipine and amlodipine); vasodilators (e.g., hydralazine), diruetics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, torsemide, furosemide, musolimine, bumetanide, triamtrenene, amiloride, spironolactone); renin inhibitors; ACE inhibitors (e.g., captopril, zofenopril, fosinopril, enalapril, ceranopril, cilazopril, delapril, pentopril, quinapril, ramipril, lisinopril); AT-1 receptor antagonists (e.g., losartan, irbesartan, valsartan); ET receptor antagonists (e.g., sitaxsentan, atrsentan and compounds disclosed in U.S. Pat. Nos. 5,612,359 and 6,043,265); Dual ET/AII antagonist (e.g., compounds disclosed in WO 00/01389); neutral endopeptidase (NEP) inhibitors; vasopepsidase inhibitors (dual NEP-ACE inhibitors) (e.g., gemopatrilat and nitrates). An exemplary antianginal agent is ivabradine.

Examples of suitable calcium channel blockers (L-type or T-type) include diltiazem, verapamil, nifedipine and amlodipine and mybefradil. Examples of suitable cardiac glycosides include digitalis and ouabain.

In one embodiment, a compound of the invention may be co-administered with one or more diuretics. Examples of suitable diuretics include (a) loop diuretics such as furosemide (such as LASIX™), torsemide (such as DEMADEX™), bemetanide (such as BUMEX™), and ethacrynic acid (such as EDECRIN™); (b) thiazide-type diuretics such as chlorothiazide (such as DIURIL™, ESIDRIX™ or HYDRODIURIL™), hydrochlorothiazide (such as MICROZIDE™ or ORETIC™), benzthiazide, hydroflumethiazide (such as SALURON™), bendroflumethiazide, methychlorthiazide, polythiazide, trichlormethiazide, and indapamide (such as LOZOL™); (c) phthalimidine-type diuretics such as chlorthalidone (such as HYGROTON™), and metolazone (such as ZAROXOLYN™); (d) quinazoline-type diuretics such as quinethazone; and (e) potassium-sparing diuretics such as triamterene (such as DYRENIUM™), and amiloride (such as MIDAMOR™ or MODURETIC™). In another embodiment, a compound of the invention may be co-administered with a loop diuretic. In still another embodiment, the loop diuretic is selected from furosemide and torsemide. In still another embodiment, one or more compounds of the invention may be co-administered with furosemide. In still another embodiment, one or more compounds of the invention may be co-administered with torsemide which may optionally be a controlled or modified release form of torsemide.

In another embodiment, a compound of the invention may be co-administered with a thiazide-type diuretic. In still another embodiment, the thiazide-type diuretic is selected from the group consisting of chlorothiazide and hydrochlorothiazide. In still another embodiment, one or more compounds of the invention may be co-administered with chlorothiazide. In still another embodiment, one or more compounds of the invention may be co-administered with hydrochlorothiazide. In another embodiment, one or more compounds of the invention may be co-administered with a phthalimidine-type diuretic. In still another embodiment, the phthalimidine-type diuretic is chlorthalidone.

Examples of suitable combination mineralocorticoid receptor antagonists include sprionolactone and eplerenone. Examples of suitable combination phosphodiesterase inhibitors include: PDE III inhibitors (such as cilostazol); and PDE V inhibitors (such as sildenafil).

The compounds of the present invention may be used in combination with cholesterol modulating agents (including cholesterol lowering agents) such as a lipase inhibitor, an HMG-CoA reductase inhibitor, an HMG-CoA synthase inhibitor, an HMG-CoA reductase gene expression inhibitor, an HMG-CoA synthase gene expression inhibitor, an MTP/Apo B secretion inhibitor, a CETP inhibitor, a bile acid absorption inhibitor, a cholesterol absorption inhibitor, a cholesterol synthesis inhibitor, a squalene synthetase inhibitor, a squalene epoxidase inhibitor, a squalene cyclase inhibitor, a combined squalene epoxidase/squalene cyclase inhibitor, a fibrate, niacin, an ion-exchange resin, an antioxidant, an ACAT inhibitor or a bile acid sequestrant or an agent such as mipomersen.

Examples of suitable cholesterol/lipid lowering agents and lipid profile therapies include: HMG-CoA reductase inhibitors (e.g., pravastatin, lovastatin, atorvastatin, simvastatin, fluvastatin, NK-104 (a.k.a. itavastatin, or nisvastatin or nisbastatin) and ZD-4522 (a.k.a. rosuvastatin, or atavastatin or visastatin)); squalene synthetase inhibitors; fibrates; bile acid sequestrants (such as questran); ACAT inhibitors; MTP inhibitors; lipooxygenase inhibitors; cholesterol absorption inhibitors; and cholesterol ester transfer protein inhibitors.

Anti-inflammatory agents also include sPLA2 and IpPLA2 inhibitors (such as darapladib), 5 LO inhibitors (such as atrelueton) and IL-1 and IL-1r antagonists (such as canakinumab).

Other atherosclerotic agents include agents that modulate the action of PCSK9, for example, bococizumab.

Cardiovascular complications of type 2 diabetes are associated with inflammation, accordingly, the compounds of the present invention may be used in combination with anti-diabetic agents, particularly type 2 anti-diabetic agents. Examples of suitable anti-diabetic agents include, for example, insulins, metfomin, DPPIV inhibitors, GLP-1 agonists, analogues and mimetics, SGLT1 and SGLT2 inhibitors. Suitable anti-diabetic agents include an acetyl-CoA carboxylase-(ACC) inhibitors such as those described in WO2009144554, WO2003072197, WO2009144555 and WO2008065508, a diacylglycerol O-acyltransferase 1 (DGAT-1) inhibitor, such as those described in WO09016462 or WO2010086820, AZD7687 or LCQ908, diacylglycerol O-acyltransferase 2 (DGAT-2) inhibitor, monoacylglycerol O-acyltransferase inhibitors, a phosphodiesterase (PDE)-10 inhibitor, an AMPK activator, a sulfonylurea (e.g., acetohexamide, chlorpropamide, diabinese, glibenclamide, glipizide, glyburide, glimepiride, gliclazide, glipentide, gliquidone, glisolamide, tolazamide, and tolbutamide), a meglitinide, an α-amylase inhibitor (e.g., tendamistat, trestatin and AL-3688), an α-glucoside hydrolase inhibitor (e.g., acarbose), an α-glucosidase inhibitor (e.g., adiposine, camiglibose, emiglitate, miglitol, voglibose, pradimicin-Q, and salbostatin), a PPARy agonist (e.g., balaglitazone, ciglitazone, darglitazone, englitazone, isaglitazone, pioglitazone and rosiglitazone), a PPAR α/γ agonist (e.g., CLX-0940, GW-1536, GW-1929, GW-2433, KRP-297, L-796449, LR-90, MK-0767 and SB-219994), a biguanide (e.g., metformin), a glucagon-like peptide 1 (GLP-1) modulator such as an agonist (e.g., exendin-3 and exendin-4), liraglutide, albiglutide, exenatide (Byetta®), albiglutide, lixisenatide, dulaglutide, semaglutide, NN-9924, TTP-054, a protein tyrosine phosphatase-1B (PTP-1B) inhibitor (e.g., trodusquemine, hyrtiosal extract, and compounds disclosed by Zhang, S., et al., Drug Discovery Today, 12(9/10), 373-381 (2007)), SIRT-1 inhibitor (e.g., resveratrol, GSK2245840 or GSK184072), a dipeptidyl peptidase IV (DPP-IV) inhibitor (e.g., those in WO2005116014, sitagliptin, vildagliptin, alogliptin, dutogliptin, linagliptin and saxagliptin), an insulin secretagogue, a fatty acid oxidation inhibitor, an A2 antagonist, a c-jun amino-terminal kinase (JNK) inhibitor, glucokinase activators (GKa) such as those described in WO2010103437, WO2010103438, WO2010013161, WO2007122482, TTP-399, TTP-355, TTP-547, AZD1656, ARRY403, MK-0599, TAK-329, AZD5658 or GKM-001, insulin, an insulin mimetic, a glycogen phosphorylase inhibitor (e.g. GSK1362885), a VPAC2 receptor agonist, SGLT2 inhibitors, such as those described in E. C. Chao et al., Nature Reviews Drug Discovery 9, 551-559 (July 2010) including dapagliflozin, canagliflozin, empagliflozin, tofogliflozin (CSG452), ASP-1941, THR1474, TS-071, ISIS388626 and LX4211 as well as those in WO2010023594, a glucagon receptor modulator such as those described in Demong, D. E. et al., Annual Reports in Medicinal Chemistry 2008, 43, 119-137, GPR119 modulators, particularly agonists, such as those described in WO2010140092, WO2010128425, WO2010128414, WO2010106457, Jones, R. M. et al., in Medicinal Chemistry 2009, 44, 149-170 (e.g. MBX-2982, GSK1292263, APD597 and PSN821), FGF21 derivatives or analogs such as those described in Kharitonenkov, A. et al., Current Opinion in Investigational Drugs 2009, 10(4)359-364, TGR5 (also termed GPBAR1) receptor modulators, particularly agonists, such as those described in Zhong, M., Current Topics in Medicinal Chemistry, 2010, 10(4), 386-396 and INT777, GPR40 agonists, such as those described in Medina, J. C., Annual Reports in Medicinal Chemistry, 2008, 43, 75-85, including but not limited to TAK-875, GPR120 modulators, particularly agonists, high affinity nicotinic acid receptor (HM74A) activators, and SGLT1 inhibitors, such as GSK1614235. A further representative listing of anti-diabetic agents that can be combined with the compounds of the present invention can be found, for example, at page 28, line 35 through page 30, line 19 of WO2011005611. Preferred anti-diabetic agents are metformin and DPP-IV inhibitors (e.g., sitagliptin, vildagliptin, alogliptin, dutogliptin, linagliptin and saxagliptin). Other antidiabetic agents could include inhibitors or modulators of carnitine palmitoyl transferase enzymes, inhibitors of fructose 1,6-diphosphatase, inhibitors of aldose reductase, mineralocorticoid receptor inhibitors, inhibitors of TORC2, inhibitors of CCR2 and/or CCR5, inhibitors of PKC isoforms (e.g. PKCα, PKCβ, PKCγ), inhibitors of fatty acid synthetase, inhibitors of serine palmitoyl transferase, modulators of GPR81, GPR39, GPR43, GPR41, GPR105, Kv1.3, retinol binding protein 4, glucocorticoid receptor, somatostain receptors (e.g. SSTR1, SSTR2, SSTR3 and SSTR5), inhibitors or modulators of PDHK2 or PDHK4, inhibitors of MAP4K4, modulators of IL1 family including ILlbeta, modulators of RXRalpha. In addition suitable anti-diabetic agents include mechanisms listed by Carpino, P. A., Goodwin, B. Expert Opin. Ther. Pat, 2010, 20(12), 1627-51.

Those skilled in the art will recognize that the compounds of this invention may also be used in conjunction with other cardiovascular or cerebrovascular treatments including PCI, stenting, drug eluting stents, stem cell therapy and medical devices such as implanted pacemakers, defibrillators, or cardiac resynchronization therapy.

The compounds of the present invention may be used in combination with neuroinflammatory and neurodegenerative agents in mammals. Examples of additional neuroinflammatory and neurodegenerative agents include antidepressants, antipsychotics, anti-pain agents, anti-Alzheimer's agents, and anti-anxiety agents. Examples of particular classes of antidepressants that can be used in combination with the compounds of the invention include norepinephrine reuptake inhibitors, selective serotonin reuptake inhibitors (SSRIs), NK-1 receptor antagonists, monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, and atypical antidepressants. Suitable norepinephrine reuptake inhibitors include tertiary amine tricyclics and secondary amine tricyclics. Examples of suitable tertiary amine tricyclics and secondary amine tricyclics include amitriptyline, clomipramine, doxepin, imipramine, trimipramine, dothiepin, butriptyline, nortriptyline, protriptyline, amoxapine, desipramine and maprotiline. Examples of suitable SSRIs include fluoxetine, fluvoxamine, paroxetine, and sertraline. Examples of monoamine oxidase inhibitors include isocarboxazid, phenelzine, and tranylcyclopramine. Examples of suitable reversible inhibitors of monoamine oxidase include moclobemide. Examples of suitable SNRIs of use in the present invention include venlafaxine. Examples of suitable atypical anti-depressants include bupropion, lithium, trazodone and viloxazine. Examples of anti-Alzheimer's agents include NMDA receptor antagonists such as memantine; and cholinesterase inhibitors such as donepezil and galantamine. Examples of suitable classes of anti-anxiety agents that can be used in combination with the compounds of the invention include benzodiazepines and serotonin 1A receptor (5-HT1A) agonists, and CRF antagonists. Suitable benzodiazepines include alprazolam, chlordiazepoxide, clonazepam, chlorazepate, diazepam, lorazepam, oxazepam, and prazepam. Suitable 5-HT1A receptor agonists include buspirone and ipsapirone. Suitable CRF antagonists include verucerfont. Suitable atypical antipsychotics include paliperidone, ziprasidone, risperidone, aripiprazole, olanzapine, and quetiapine. Suitable nicotine acetylcholine agonists include CP-601927 and varenicline. Anti-pain agents include pregabalin, gabapentin, clonidine, neostigmine, baclofen, midazolam, ketamine and ziconotide.

The present invention further comprises kits that are suitable for use in performing the methods of treatment described above. In one embodiment, the kit contains a first dosage form comprising one or more of the compounds of the present invention and a container for the dosage, in quantities sufficient to carry out the methods of the present invention.

In another embodiment, the kit of the present invention comprises one or more compounds of the invention.

The present invention further comprises intermediate compounds useful in the synthesis of the compounds of the invention, including salts and/or tautomers thereof.

### General Synthetic Schemes

The compounds of Formula Ia may be prepared by the methods described below, together with synthetic methods known in the art of organic chemistry, or modifications and transformations that are familiar to those of ordinary skill in the art. The starting materials used herein are commercially available or may be prepared by routine methods known in the art [such as those methods disclosed in standard reference books such as the Compendium of Organic Synthetic Methods, Vol. I-XII (published by Wiley-lnterscience)]. Preferred methods include, but are not limited to, those described below.

During any of the following synthetic sequences it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981; T. W. Greene and P. G. M. Wuts, Protective Groups in Organic Chemistry, John Wiley & Sons, 1991; and T. W. Greene and P. G. M. Wuts, Protective Groups in Organic Chemistry, John Wiley & Sons, 1999, which are hereby incorporated by reference.

Compounds of Formula I, or their pharmaceutically acceptable salts, can be prepared according to the reaction Schemes discussed herein below. Unless otherwise indicated, the substituents in the Schemes are defined as above. Isolation and purification of the products is accomplished by standard procedures, which are known to a chemist of ordinary skill.

It will be understood by one skilled in the art that the various symbols, superscripts and subscripts used in the schemes, methods and examples are used for convenience of representation and/or to reflect the order in which they are introduced in the schemes, and are not intended to necessarily correspond to the symbols, superscripts or subscripts in the appended claims. Additionally, one skilled in the art will recognize that in many cases, these compounds will be mixtures and diastereomers and/or enantiomers that may be separated at various stages of the synthetic schemes using conventional techniques, such as, but not limited to, crystallization, normal-phase chromatography, reversed phase chromatography and chiral chromatography, to afford single enantiomers. The schemes are representative of methods useful in synthesizing the compounds of the present invention. They are not to constrain the scope of the invention in any way.

Methods to prepare compounds of the invention are similar to those described in U.S. patent application Ser. No. 14/678,114 filed on Apr. 3, 2015 (PCT/IB2015/052251) and is hereby incorporated in its entirety for all purposes. In addition, the methods to prepare compounds of the invention are described below in the following schemes.

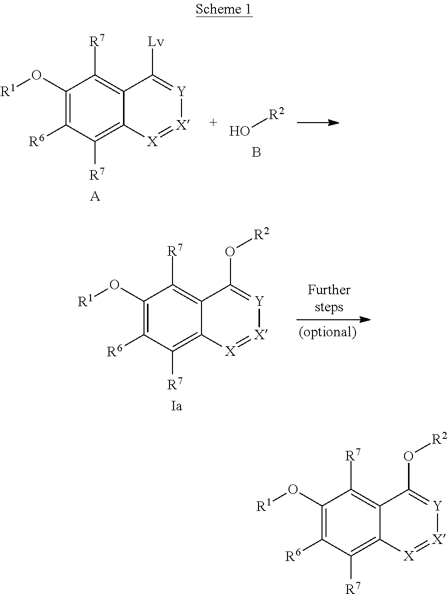

Scheme 1 illustrates a method for preparing compounds of Formula Ia. A compound of Formula A, in which Lv is a displaceable leaving group (such as chloro or fluoro, for example) is treated with a compound of Formula B (for example, as commercially available) to furnish a product of Formula Ia. The reaction is typically carried out in the presence of a suitable base such as cesium carbonate, potassium tert-butoxide, sodium hydride or potassium hexamethyldisilazide in a suitable solvent or solvent mixture, such as THF or dimethylformamide. The compounds of Formula A may be prepared as described in the subsequent schemes. The compounds of formula R²—OH may be obtained from commercial vendors or prepared by methods reported in the chemical literature.

If desired, further transformations may be effected upon the compound of Formula Ia. For example, the compound of Formula Ia wherein R⁶═CN may be subjected to a nitrile hydrolysis reaction to provide a compound of Formula Ia in which R⁶═CONH₂. The reaction may be carried out in a variety of ways known to one skilled in the art, for example by the use of acids or bases, optionally in the presence of an oxidant such as hydrogen peroxide, or by using chemical or enzymatic catalysts. In other cases, the compound of Formula Ia may be further treated with reagents, such as acids, to cleave protecting groups, such as t-butoxycarbonyl groups, and/or with other reagents to derivatize functional groups such as carboxyl, amino, or hydroxyl groups.

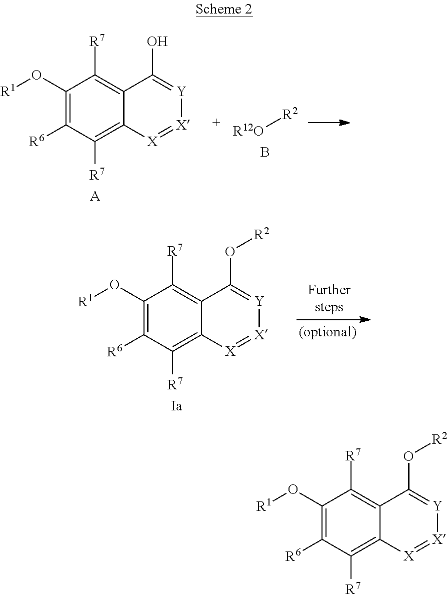

Scheme 2 illustrates another method for the preparation of compounds of Formula Ia, particularly suited to those instances in which X, X′ and Y in the compound of Formula A all are carbon. This method provides for the alkylation of a compound of Formula A with a compound of Formula B (wherein the R¹²O— group is hydroxyl or a sulfonate ester such as p-toluenesulfonate or methanesulfonate; for example, (or as commercially available), using methods known to those skilled in the art, to furnish a product of Formula Ia. For example, this reaction may be carried out by treating a compound of Formula A with a compound of Formula B (R¹²═H) in the presence of triphenylphosphine and an azodicarboxylate ester (“Mitsunobu reaction”) in a suitable solvent such as THF. Alternatively, the alkylation of a compound of Formula A may be effected using a compound of Formula B (R¹²O═TsO or other sulfonate ester) in the presence of a base such as cesium carbonate, in a suitable solvent such as THF or dimethylformamide.

If desired, further transformations may be effected upon the compound of Formula Ia. For example, the compound of Formula Ia wherein R⁶═CN may be subjected to a nitrile hydrolysis reaction to provide a compound of Formula Ia in which R⁶═CONH₂. The reaction may be carried out in variety of ways known to one skilled in the art, for example by the use of acids or bases, optionally in the presence of an oxidant such as hydrogen peroxide, or by using chemical or enzymatic catalysts. In other cases, the compound of Formula Ia may be further treated with reagents, such as acids, to cleave protecting groups, such as t-butoxycarbonyl groups, and/or with other reagents to derivatize functional groups such as carboxyl, amino, or hydroxyl groups.

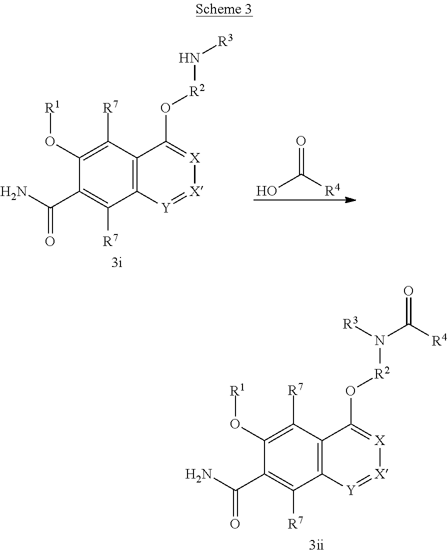

If desired, products 3i may be further modified by conversion to amines by deprotection of common protecting groups (for example, where R³═BOC or benzyl) and by further functionalization. For instance an amine may be revealed by removal of protecting groups on the amine partners, such as t-butyloxycarbonyl groups by the action of dry acid, such as triflouoroacetic acid or dry HCl in a suitable solvent such as dioxane. The resulting amine may be a final product or further modified conversion to amides using available carboxylic acids (R⁴CO₂H) and a suitable coupling agent such as BOP or HATU to couple the acid and amine partner. In some cases the amide partner R⁴CO₂H may itself have a protecting group such as a t-butyloxycarbonyl group and may be subjected to deprotection with dry acid as referred to above to generate final products of the invention. Additional functionalization may be introduced, such as sulfonyl groups through the reaction of amines with sulfonyl chlorides and an appropriate base such as pyridine or tertiary amines in dichloromethane or THF.

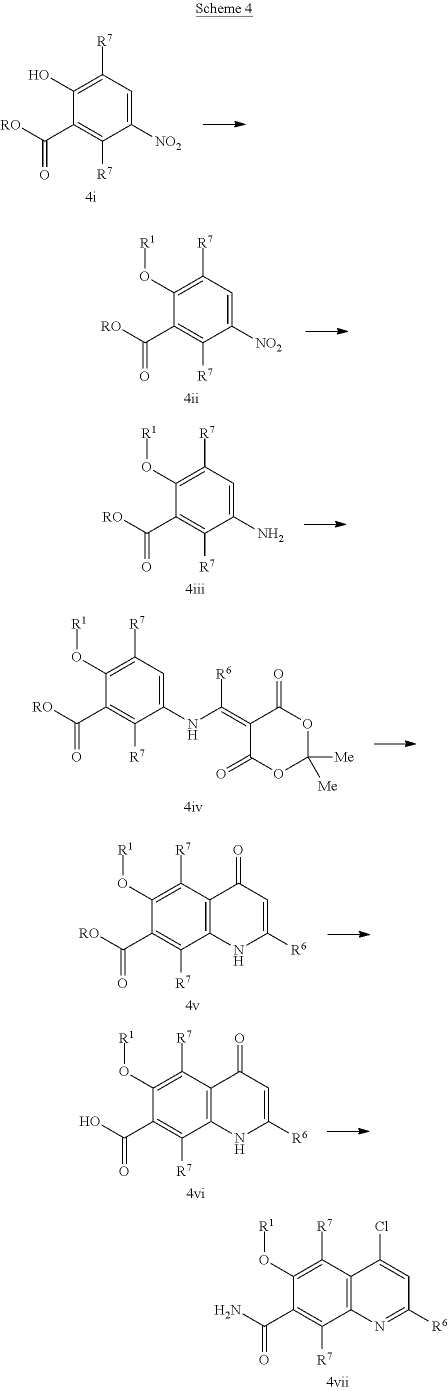

Scheme 4 illustrates an approach to prepare quinoline compounds of the invention. Alkylation of phenolic compounds such as a 2-alkylcarboxy-4-nitrophenol 4i through alkylation with base and an appropriate alkylating agent, a halide or sulfonyl ester of an alcohol or by reaction with DIAD and triphenylphosphine and an alcohol (“Mitsunobu reaction”) can be used to prepare ether compounds 4ii. Nitro reduction by methods known to those skilled in the art gives compounds 4iii followed by reaction of Meldrum's acid and trialkoxyorthoesters warmed in alcoholic solvents such as ethanol generates enamides of Meldrum's acid such as compounds 4iv which may have an R⁶ substitutent or a hydrogen atom. When subjected to high temperature typically in an inert solvent such as Dowtherm at 280° C., cyclization of a compound such as 4iv to a quinolone intermediate compound such as 4v can occur. This compound may be saponified to afford an acid quinolone 4vi, then treated with an appropriate chlorinating agent such as phosphorus oxychloride at elevated temperature such as at the reflux temperature to generate the dichloride. This material may be treated with sources of ammonia and a coupling reagent in a solvent such as methanol to yield an amide quinoline chloride compound 4vii shown. This compound may be further elaborated as described in Scheme 1 and in the methods of the experimental section.

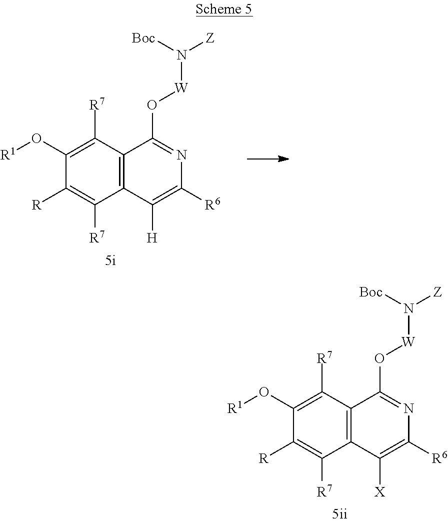

Certain compounds of the invention may be accessed by halogenation and if desired by subsequent functionalization. For instance compounds such as 5i may be treated with a reagent such as NCS or NBS to generate the corresponding halogenated compounds 5ii where X is a halogen such as chlorine or bromine. Such compounds may be further functionalized as described elsewhere and in the experimental section. With catalysis by metals such as palladium, the halide may be converted to other functional groups X such as nitriles and alkyl or aryl groups such as with Pd(PPh₃)Cl₂, base such as K₂CO₃ and Zn(CN)₂ or alkyltrifluoroborate potassium salt in a solvent such as degassed MeCN or other solvents such as DMF or water as may be common for such transformations and heated to elevated temperatures 70-125° C. The conversion of the X group to a nitrile or other groups may also be performed when the R group is an amide.

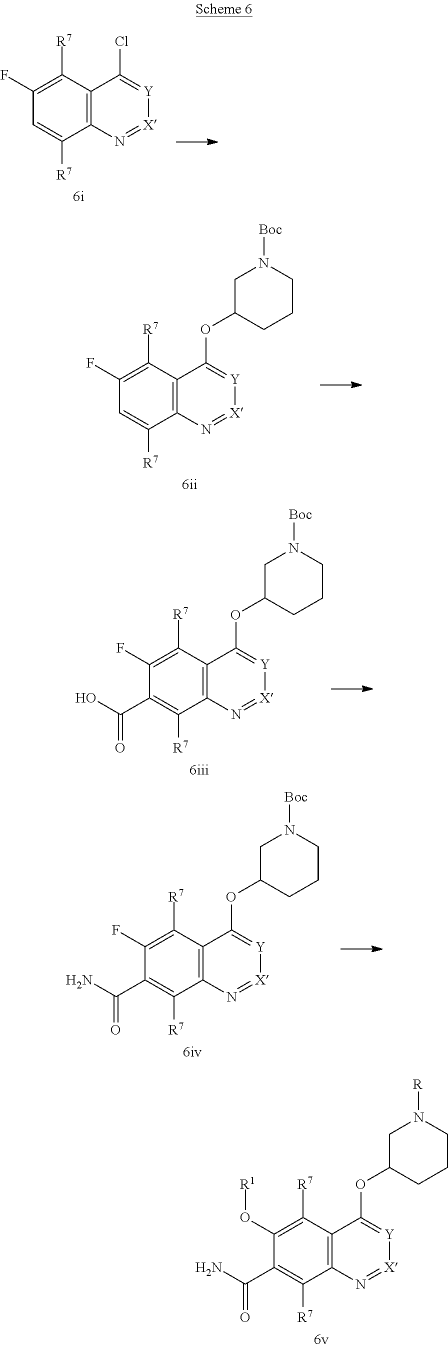

Scheme 6 illustrates an additional method to prepare compounds 6v of the invention. Beginning with a compound such as 6i, an S_{N}Ar reaction with an alcohol and a base in suitable polar solvents as described in Scheme 1 may deliver an ether compound 6ii. Directed metalation using a strong base, such as lithium diisopropyl amide in a solvent such as THF at low temperature, followed by treatment with sources of dry carbon dioxide may provide a carboxylic acid 6iii. This compound may be converted to its corresponding amide by a number of means known to those in the art, including treatment with BOP and ammonium hydroxide to give compound 6iv. S_{N}Ar reaction of 6iv with alcohols R¹OH and base such as KO-t-Bu in DMF leads to compounds such as 6v that can be further elaborated as described elsewhere. In the case of R¹ being a removable group, for instance a benzyl group, conversion to the R¹═H phenol of 6v may be accomplished by standard reaction methodology, by for instance hydrogenation with hydrogen gas and a suitable catalyst, such as palladium on carbon. Further elaboration of this phenol may be accomplished as described elsewhere herein.

### Experimental Procedures and Working Examples

The following illustrate the synthesis of various compounds of the present invention.

Additional compounds within the scope of this invention may be prepared using the methods illustrated in these Examples, either alone or in combination with techniques generally known in the art.

It will be understood that the intermediate compounds of the invention depicted above are not limited to the particular enantiomer shown, but also include all stereoisomers and mixtures thereof. It will also be understood that compounds of Formula Ia can include intermediates of compounds of Formula Ia.

### Experimental Procedures

Experiments were generally carried out under inert atmosphere (nitrogen or argon), particularly in cases where oxygen- or moisture-sensitive reagents or intermediates were employed. Commercial solvents and reagents were generally used without further purification, including anhydrous solvents where appropriate (generally Sure-Seal™ products from the Aldrich Chemical Company, Milwaukee, Wis.). Products were generally dried under vacuum before being carried on to further reactions or submitted for biological testing. Mass spectrometry data is reported from either liquid chromatography-mass spectrometry (LCMS), atmospheric pressure chemical ionization (APCI) or gas chromatography-mass spectrometry (GCMS) instrumentation. Chemical shifts for nuclear magnetic resonance (NMR) data are expressed in parts per million (ppm, 8) referenced to residual peaks from the deuterated solvents employed.

For syntheses referencing procedures in other Examples or Methods, reaction conditions (length of reaction and temperature) may vary. In general, reactions were followed by thin layer chromatography and/or liquid chromatography-mass spectrometry, and subjected to work-up when appropriate. It will be recognized by one skilled in the art that purifications may vary between experiments: in general, sorbents, solvents and the solvent ratios used for eluants/gradients were chosen to provide appropriate R_{f}s or retention times. It will also be recognized by one skilled in the art that HPLC purifications may be effected in a variety of ways, including the use of normal stationary phases, reverse stationary phases, chiral stationary phases, and supercritical eluants. The appropriate choices of conditions for chromatographic and HPLC purifications will be discerned by one skilled in the art.

The following Preparations describe the preparation of certain intermediates used in the Methods and Examples that follow. The following Preparations, Methods and Examples are intended to illustrate particular embodiments of the invention and preparations thereto and are not intended to limit the specification, including the claims, in any manner. Unless noted otherwise, all reactants were obtained commercially.

In the non-limiting Examples and Preparations that are set out later in the description and in the aforementioned Schemes, the following abbreviations, definitions and analytical procedures may be referred to:
AcOH is acetic acid;
aq is aqueous;
BOC, Boc is tert-butyloxycarbonyl;
BOP is (benzotriazol-1-yloxy)tris(dimethylamino)phosphonium hexafluorophosphate;
br is broad;
° C. is degrees Celsius;
CDCl₃ is deuterochloroform;
δ is chemical shift;
d is doublet;
DCM is dichloromethane; methylene chloride;
DIAD is diisopropylazodicarboxylate;
DMF is dimethylformamide;
DMSO is dimethyl sulphoxide;
EtOAc is ethyl acetate;
EtOH is ethanol;
g is gram;
h is hour;
H₂O₂ is hydrogen peroxide;
H₂SO₄ is sulfuric acid;
HATU is 1-[bis(dimethylamino)methylene]-1H-1,2,3-triazolo[4,5-b]pyridinium 3-oxid hexafluorophosphate;
HCl is hydrochloric acid;
HPLC is high performance liquid chromatography;
K₂CO₃ is potassium carbonate;
L is liter;
LCMS is liquid chromatography mass spectrometry (Rt=retention time);
LDA is lithium diisopropylamide;
m is multiplet;
M is molar;
Me is methyl;
MeCN is acetonitrile;
MeOH is methanol;
MTBE is tert-butylmethyl ether;
mg is milligram;
MHz is megahertz;
min is min;
mL is milliliter;
mmol is millimole;
mol is mole;
MS m/z is mass spectrum peak;
n-BuLi is n-butyl lithium;
NaH is sodium hydride;
NaHCO₃ is sodium hydrogencarbonate;
NaOH is sodium hydroxide;
Na₂SO₄ is sodium sulfate;
Na₂S₂O₃ is sodium thiosulfate;
NBS is N-bromosuccinimide;
NCS is N-chlorosuccinimide;
NH₃ or NH₄OH is ammonia or ammonium hydroxide;
NMM N-methylmorpholine;
NMP is N-methylpyrrolidinone;
NMR is nuclear magnetic resonance;
pH is power of hydrogen;
POCl₃ is phosphorus oxychloride;
Pd₂(dba)₃ is tris(dibenzylideneacetone)dipalladium(0);
Pd(PPh₃)Cl₂ is bis(triphenylphosphine)palladium(II) dichloride;
ppm is parts per million;
q is quartet;
Rt is retention time;
s is singlet;
t is triplet;
tBuOK is potassium tert-butoxide;
TEA is triethylamine;
TFA is trifluoroacetic acid;
THF is tetrahydrofuran;
μL is microliter;
μmol is micromol and
X-Phos is 2-dicyclohexylphosphino-2′,4′,6′-triisopropylbiphenyl

¹H Nuclear magnetic resonance (NMR) spectra were in all cases consistent with the proposed structures. Characteristic chemical shifts (δ) are given in ppm downfield from tetramethylsilane (for ¹H-NMR) using conventional abbreviations for designation of major peaks: e.g. s, singlet; d, doublet; t, triplet; q, quartet; m, multiplet; br, broad. The following abbreviations have been used for common solvents: CDCl₃, deuterochloroform; d₆-DMSO, deuterodimethylsulphoxide; and CD₃OD, deuteromethanol.

Mass spectra, MS (m/z), were recorded using either electrospray ionization (ESI) or atmospheric pressure chemical ionization (APCI). Where relevant and unless otherwise stated the m/z data provided are for isotopes ¹⁹F, ³⁵Cl, ⁷⁹Br and ¹²⁷I.

### Library Protocol 1

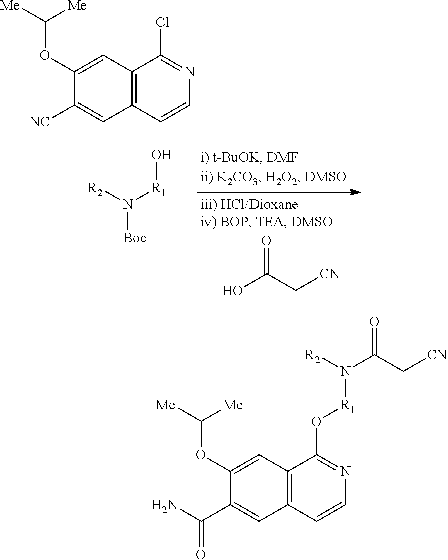

Step 1: To the appropriate amino alcohol (150 μmol) was added 1-chloro-7-isopropoxyisoquinoline-6-carbonitrile (Preparation 35, 1000 μL of a 0.15M solution in DMF, 150 μmol) followed by t-BuOK (33.6 mg, 300 μmol). The reaction vessels were capped and shaken at 100° C. for 16 h. The reaction mixtures were concentrated in vacuo and treated with water (1 mL). The mixtures were extracted with EtOAc (3×1 mL) and the organic layers were collected, washed with brine (2 mL) dried over Na₂SO₄ and concentrated in vacuo. The residues were used directly in the next step without further purification.

Step 2: To solutions of the residues from Step 1 in DMSO (1100 μL) was added K₂CO₃ (90 mg, 450 μmol) followed by H₂O₂(40% in water, 400 μL). The reaction vessels were capped and shaken at 60° C. for 20 min. The reaction mixtures were diluted with water (1 mL) and treated with saturated aq Na₂S₂O₃ solution (1 mL). The mixtures were extracted with EtOAc (3×1.5 mL), and the organic layers were collected, washed with water (1.5 mL), brine (1.5 mL), dried over Na₂SO₄ and concentrated in vacuo. The residues were used directly in the next step without further purification.

Step 3: To solutions of the residues from Step 2 in DCM (2000 μL) was added 4M HCl in dioxane (400 μL). The reaction vessels were capped and shaken at 30° C. for 1.5 h. The reaction mixtures were concentrated in vacuo and the residues were used directly in the next step without further purification.

Step 4: To the residues from Step 3 were added TEA (42 μL, 300 μmol), cyanoacetic acid (500 μL of a 0.25M solution in DMSO, 125 μmol) and BOP (500 μL of a 0.3M solution in DMSO, 150 μmol). Additional TEA (42 μL, 300 μmol) was added and the reaction mixtures were capped and shaken at 30° C. for 16 h. The reaction mixtures were dissolved in DMSO, filtered and purified using preparative HPLC as described below.

Purification Method A: Agella Venusil ASB C18 150×21.2 mm, 5 μm; between 23-63% MeCN in water with 0.225% formic acid. Gradient time=10 min; flow rate=30 mL/min.

Purification Method B: Kromasil Eternity-5 C18 150×30 mm, 5 μm; between 24-54% MeCN in water with 0.225% formic acid. Gradient time=10 min; flow rate=30 mL/min.

LCMS Method 1: XBRIDGE 50×2.1 mm, 5 μm; mobile phase A: 0.0375% TFA in water; mobile phase B: 0.01875% TFA in MeCN; gradient from 1% B to 5% B at 0.60 mins, further to 100% B at 4.00 mins and finally returning to 1% B at 4.30-4.70 mins; flow rate 0.8 mL/min.

LCMS Method 2: XBRIDGE 50×2.1 mm, 5 μm; mobile phase A: 0.05% NH₄OH in water; mobile phase B: 100% MeCN; gradient from 5% B to 100% B at 3.40 mins, hold at 100% B to 4.20 mins and finally returning to 5% B at 4.21-4.70 mins; flow rate 0.8 mL/min.

LCMS Method 3: Thermo Hypersil Aquasil C18 50×2.1 mm, 5 μm; mobile phase A: 10 mM NH₄OAc in 95% water/5% MeCN; mobile phase B: 10 mM NH₄OAc in 95% MeCN/5% water; gradient of 0% B in 2.5 mins; flow rate 0.8 mL/min.

Examples 1-3 were prepared according to the method described for Library Protocol 1 Steps 1-4 using 1-chloro-7-isopropoxyisoquinoline-6-carbonitrile (Preparation 35) and the appropriate aminoalcohol as described below. The examples were isolated as formate salts.

| Ex. | | | |
|---|---|---|---|
| No. | Name | Structure | Analytical Data/Starting Material |
| 1 | 1-{[(3S)-1- (cyanoacetyl)pyrrolidin- 3-yl]oxy}-7-(propan-2- yloxy)isoquinoline-6- carboxamide formate | 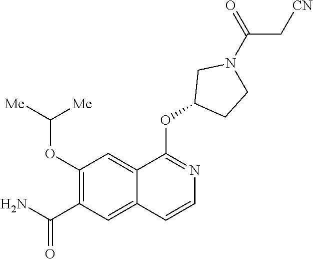 [SMILES: CC(C)Oc1cc2c(O[C@H]3CCN(C(=O)CC#N)C3)nccc2cc1C(N)=O] | MS m/z 383 [M + H]⁺ LCMS Rt = 2.52 min tert-butyl (S)-3-hydroxypyrrolidine-1- carboxylate. Purification Method A, LCMS Method 1. |
| 2 | 1-{[(3R)-1- (cyanoacetyl)pyrrolidin- 3-yl]oxy}-7-(propan-2- yloxy)isoquinoline-6- carboxamide formate | 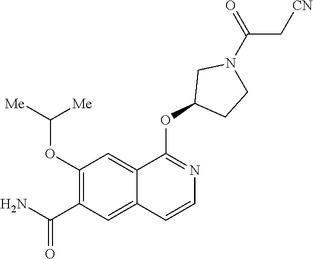 [SMILES: CC(C)Oc1cc2c(O[C@@H]3CCN(C(=O)CC#N)C3)nccc2cc1C(N)=O] | MS m/z 383 [M + H]⁺ LCMS Rt = 2.29 min tert-butyl (R)-3-hydroxypyrrolidine-1- carboxylate. Purification Method A, LCMS Method 2. |
| 3 | 1-{[(3R,4S)-1- (cyanoacetyl)-3- fluoropiperidin-4- yl]oxy}-7-(propan-2- yloxy)isoquinoline-6- carboxamide formate | 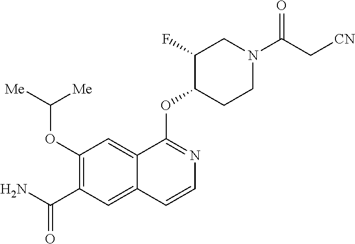 [SMILES: CC(C)Oc1cc2c(O[C@H]3CCN(C(=O)CC#N)C[C@H]3F)nccc2cc1C(N)=O] | MS m/z 415 [M + H]⁺ LCMS Rt = 2.72 min tert-butyl (3R,4S)-3-fluoro-4- hydroxypiperidine-1-carboxylate. Purification Method B, LCMS Method 1. |

### Library Protocol 2

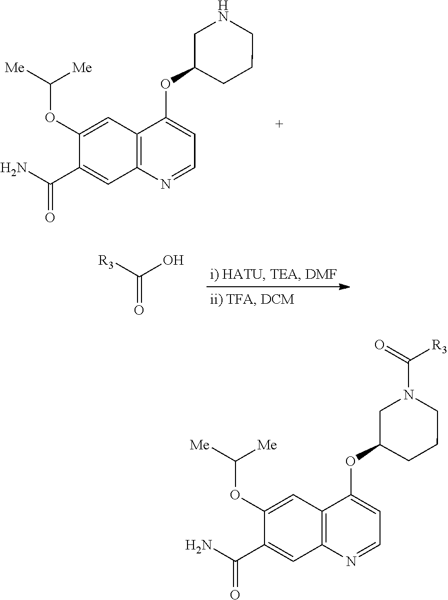

Step 1: To (R)-4-(piperidin-3-yloxy)-6-(propan-2-yloxy)quinoline-7-carboxamide (Example 58, 200 μL of a 0.625M solution in DMF, 125 μmol) was added the desired carboxylic acid (200 μL of a 0.5M solution in DMF, 100 μmol) followed by TEA (30 μL, 216 μmol) and HATU (200 μL of a 0.5M solution in DMF, 100 μmol). The reaction vessels were capped and shaken at 30° C. for 16 h. The reaction mixtures were concentrated in vacuo and purified using preparative HPLC as described below. The residues were treated with a solution of TFA in DCM (v:v 1:10, 2 mL), capped and shaken at 30° C. for 30 min. The reactions were concentrated in vacuo to afford the desired compounds as described below.

Purification Method A: Boston Symmetrix C18 ODS-R 150×30 mm, 5 μm; organic mobile phase: MeCN; aq mobile phase: water with 0.225% formic acid. Gradient time=9 min; flow rate=30 mL/min.

Purification Method B: DIKMA Diamonsil C18 200×20 mm, 5 μm; organic mobile phase: MeCN; aq mobile phase: water with 0.225% formic acid. Gradient time=8 min; flow rate=35 mL/min.

Purification Method C: Phenomenex Synergi C18 250×21.2 mm, 4 μm; organic mobile phase: MeCN; aq mobile phase: water with 0.225% formic acid. Gradient time=12 min; flow rate=25 mL/min.

LCMS Method 1: XBRIDGE 50×2.1 mm, 5 μm; mobile phase A: 0.0375% TFA in water; mobile phase B: 0.01875% TFA in MeCN; gradient from 1% B to 5% B at 0.60 mins, further to 100% B at 4.00 mins and finally returning to 1% B at 4.30-4.70 mins; flow rate 0.8 mL/min.

LCMS Method 2: XBRIDGE 50×2.1 mm, 5 μm; mobile phase A: 0.05% NH₄OH in water; mobile phase B: 100% MeCN; gradient from 5% B to 100% B at 3.40 mins, hold at 100% B to 4.20 mins and finally returning to 5% B at 4.21-4.70 mins; flow rate 0.8 mL/min.

Examples 4-15 were prepared according to the method described for Library Protocol 2 Steps 1-2 using (R)-4-(piperidin-3-yloxy)-6-(propan-2-yloxy)quinoline-7-carboxamide (Example 58) and the appropriate carboxylic acid as described below. The organic gradient used in preparative HPLC purification is also noted. The examples were isolated as formate salts.

| Ex. | | | |
|---|---|---|---|
| No. | Name | Structure | Analytical Data/Starting Material |
| 4 | 4-{[(3R)-1- (hydroxyacetyl)piperidin- 3-yl]oxy}-6-(propan-2- yloxy)quinoline-7- carboxamide formate | 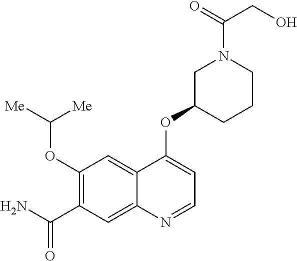 [SMILES: CC(C)Oc1cc2c(O[C@@H]3CCCN(C(=O)CO)C3)ccnc2cc1C(N)=O] | MS m/z 388 [M + H]⁺ LCMS Rt = 2.06 min 2-hydroxyacetic acid. Purification Method A with 2-32% organic gradient. LCMS Method 1. |
| 5 | 4-{[(3R)-1- (difluoroacetyl)piperidin-3- yl]oxy}-6-(propan-2- yloxy)quinoline-7- carboxamide formate | 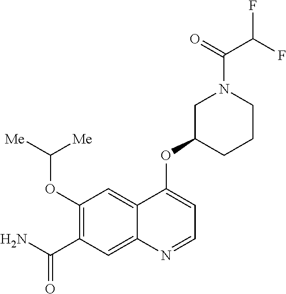 [SMILES: CC(C)Oc1cc2c(O[C@@H]3CCCN(C(=O)C(F)F)C3)ccnc2cc1C(N)=O] | MS m/z 408 [M + H]⁺ LCMS Rt = 2.28 min 2,2-difluoroacetic acid. Purification Method A with 7-37% organic gradient. LCMS Method 1. |
| 6 | 4-{[(3R)-1-glycylpiperidin- 3-yl]oxy}-6-(propan-2- yloxy)quinoline-7- carboxamide formate | 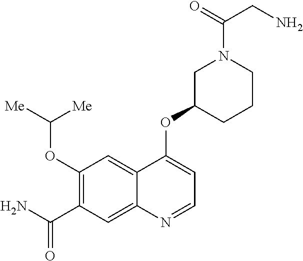 [SMILES: CC(C)Oc1cc2c(O[C@@H]3CCCN(C(=O)CN)C3)ccnc2cc1C(N)=O] | MS m/z 387 [M + H]⁺ LCMS Rt = 2.20 min (tert-butoxycarbonyl)glycine. Purification Method A with 13-43% organic gradient. LCMS Method 2. |
| 7 | 4-{[(3R)-1-(L- alanyl)piperidin-3-yl]oxy}- 6-(propan-2- yloxy)quinoline-7- carboxamide formate | 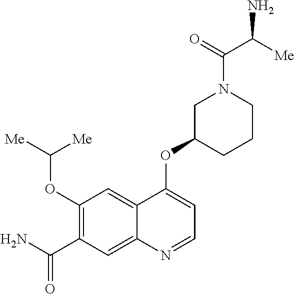 [SMILES: CC(C)Oc1cc2c(O[C@@H]3CCCN(C(=O)[C@H](C)N)C3)ccnc2cc1C(N)=O] | MS m/z 401 [M + H]⁺ LCMS Rt = 2.24 min (tert-butoxycarbonyl)-L-alanine. Purification Method A with 15-45% organic gradient. LCMS Method 2. |
| 8 | 4-{[(3R)-1- (methoxyacetyl)piperidin- 3-yl]oxy}-6-(propan-2- yloxy)quinoline-7- carboxamide formate | 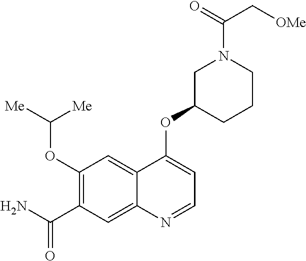 [SMILES: COCC(=O)N1CCC[C@@H](Oc2ccnc3cc(C(N)=O)c(OC(C)C)cc23)C1] | MS m/z 402 [M + H]⁺ LCMS Rt = 2.26 min 2-methoxyacetic acid. Purification Method B with 12-42% organic gradient. LCMS Method 1. |
| 9 | 6-(propan-2-yloxy)-4- {[(3R)-1- (trifluoroacetyl)piperidin- 3-yl]oxy}quinoline-7- carboxamide formate | 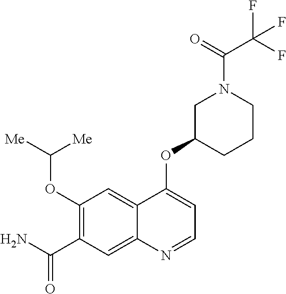 [SMILES: CC(C)Oc1cc2c(O[C@@H]3CCCN(C(=O)C(F)(F)F)C3)ccnc2cc1C(N)=O] | MS m/z 426 [M + H]⁺ LCMS Rt = 2.51 min Trifluoroacetic acid. Purification Method A with 14-44% organic gradient. LCMS Method 1. |
| 10 | 4-{[(3R)-1- propanoylpiperidin-3- yl]oxy}-6-(propan-2- yloxy)quinoline-7- carboxamide formate | 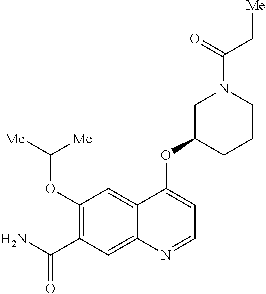 [SMILES: CCC(=O)N1CCC[C@@H](Oc2ccnc3cc(C(N)=O)c(OC(C)C)cc23)C1] | MS m/z 386 [M + H]⁺ LCMS Rt = 2.35 min Propionic acid. Purification Method B with 15-45% organic gradient. LCMS Method 1. |
| 11 | 4-{[(3R)-1-(1,3-oxazol-5- ylcarbonyl)piperidin-3- yl]oxy}-6-(propan-2- yloxy)quinoline-7- carboxamide formate | 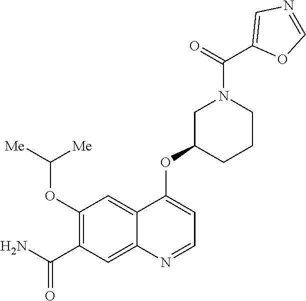 [SMILES: CC(C)Oc1cc2c(O[C@@H]3CCCN(C(=O)c4cnco4)C3)ccnc2cc1C(N)=O] | MS m/z 425 [M + H]+ LCMS Rt = 2.12 min Oxazole-5-carboxylic acid. Purification Method A with 3-33% organic gradient. LCMS Method 1. |
| 12 | Racemic-4-{[(3R)-1-(2- amino-3-hydroxy-2- methylpropanoyl)piperidin- 3-yl]oxy}-6-(propan-2- yloxy)quinoline-7- carboxamide formate | 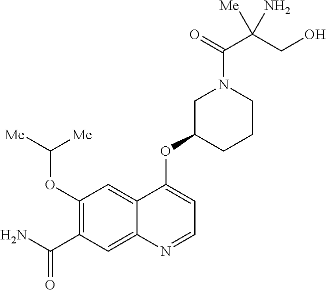 [SMILES: CC(C)Oc1cc2c(O[C@@H]3CCCN(C(=O)C(C)(N)CO)C3)ccnc2cc1C(N)=O] | MS m/z 431 [M + H]⁺ LCMS Rt = 2.078 min 2-([tert-butoxycarbonyl]amino)-3- hydroxy-2-methylpropanoic acid. Purification Method A with 12-42% organic gradient. LCMS Method 1. |
| 13 | 4-({(3R)-1-[(2S)-2- hydroxypropanoyljpiperidin- 3-yl}oxy)-6-(propan-2- yloxy)quinoline-7- carboxamide formate | 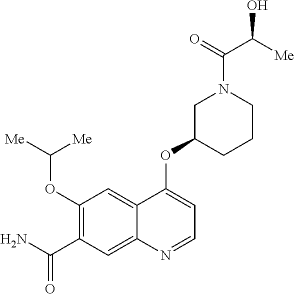 [SMILES: CC(C)Oc1cc2c(O[C@@H]3CCCN(C(=O)[C@H](C)O)C3)ccnc2cc1C(N)=O] | MS m/z 402 [M + H]⁺ LCMS Rt = 2.12 min (S)-2-hydroxypropanoic acid. Purification Method A with 3-33% organic gradient. LCMS Method 1. |
| 14 | 4-{[(3R)-1-(1H-imidazol-4- ylcarbonyl)piperidin-3- yl]oxy}-6-(propan-2- yloxy)quinoline-7- carboxamide formate | 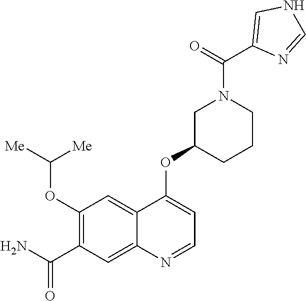 [SMILES: CC(C)Oc1cc2c(O[C@@H]3CCCN(C(=O)c4c[nH]cn4)C3)ccnc2cc1C(N)=O] | MS m/z 424 [M + H]⁺ LCMS Rt = 2.11 min 1H-imidazole-4-carboxylic acid. Purification Method C with 1-29% organic gradient. LCMS Method 2. |
| 15 | 4-{[(3R)-1-{[1- (aminomethyl)cyclopropyl] carbonyl}piperidin-3- yl]oxy}-6-(propan-2- yloxy)quinoline-7- carboxamide formate | 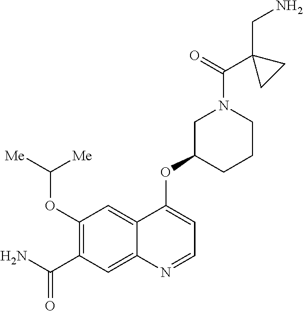 [SMILES: CC(C)Oc1cc2c(O[C@@H]3CCCN(C(=O)C4(CN)CC4)C3)ccnc2cc1C(N)=O] | MS m/z 427 [M + H]⁺; LCMS Rt = 2.10 min; 1-({[tert- butoxycarbonyl]amino}methyl) cyclopropane-1-carboxylie acid. Purification Method A with 15-45% organic gradient. LCMS Method 1. |

### Library Protocol 3

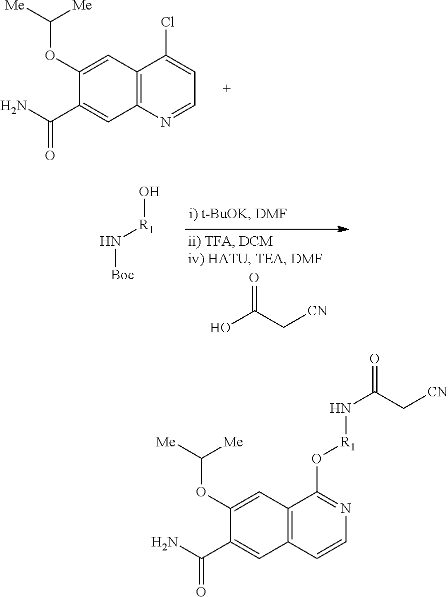

Step 1: To the appropriate N—BOC-aminoalcohol (250 μmol) was added a 0.125M solution of 4-chloro-6-isopropoxyquinoline-7-carboxamide in DMF (Preparation 38, 1 mL, 125 μmol) followed by a 1M solution of t-BuOK in THF (250 μL, 250 μmol). The reaction vessels were capped and shaken at 30° C. for 16 h. The reactions were quenched by the addition of water (100 μL), lyophilized and used directly in the next step.

Step 2: To the residue from Step 1 was added a solution of TFA in DCM (1 mL, v:v 1:8) and the reaction vessels were capped and shaken at 30° C. for 2 h. The reaction was concentrated in vacuo and used directly in the next step.

Step 3: To the residue from Step 2 was added cyanoacetic acid (10.6 mg, 125 μmol) followed by DMF (1 mL), HATU (47.5 mg, 125 μmol) and TEA (35 μL, 250 μmol). The reaction vessels were capped and shaken at 30° C. for 16 h. The reaction mixture was concentrated in vacuo and purified directly used preparative HPLC as described below.

Purification Method: Agella Venusil ASB C18 150×21.2 mm, 5 μm; between 23-63% MeCN in water with 0.225% formic acid. Gradient time=10 min; flow rate=30 mL/min. Organic gradients were between 5-47%.

LCMS Method 1: XBRIDGE 50×2.1 mm, 5 μm; mobile phase A: 0.0375% TFA in water; mobile phase B: 0.01875% TFA in MeCN; gradient from 1% B to 5% B at 0.60 min, further to 100% B at 4.00 mins and finally returning to 1% B at 4.30-4.70 min; flow rate 0.8 mL/min.

Examples 16-18 were prepared according to the method described for Library Protocol 3 using 4-chloro-6-isopropoxyquinoline-7-carboxamide (Preparation 38) and the appropriate alcohol. The Examples were isolated as formate salts.

| Ex. | | | |
|---|---|---|---|
| No. | Name | Structure | Data/Starting Material |
| 16 | 4-{[(3R,5R)-1- (cyanoacetyl)-5- methylpyrrolidin-3- yl]oxy}-6-(propan-2- yloxy)quinoline-7- carboxamide formate | 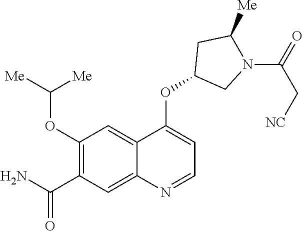 [SMILES: CC(C)Oc1cc2c(O[C@@H]3C[C@@H](C)N(C(=O)CC#N)C3)ccnc2cc1C(N)=O] | MS m/z 397 [M + H]⁺ LCMS Rt = 2.11 min (2R, 4R)-N-BOC-4-hydroxy-2- methylpyrrolidine. |
| 17 | 4-{[(2R,3S)-1- (cyanoacetyl)-2- methylpyrrolidin-3- yl]oxy}-6-(propan-2- yloxy)quinoline-7- carboxamide formate | 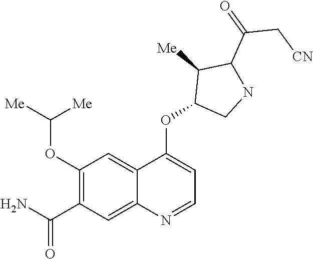 [SMILES: CC(C)Oc1cc2c(O[C@H]3CNC(C(=O)CC#N)[C@@H]3C)ccnc2cc1C(N)=O] | MS m/z 397 [M + H]⁺ LCMS Rt = 2.07 min (2R, 3S)-N-BOC-3-hydroxy-2- methylpyrrolidine (WO 2005051391). |
| 18 | 4-{[(3R,4S)-1- (cyanoacetyl)-3- fluoropiperidin-4-yl]oxy}- 6-(propan-2- yloxy)quinoline-7- carboxamide formate | 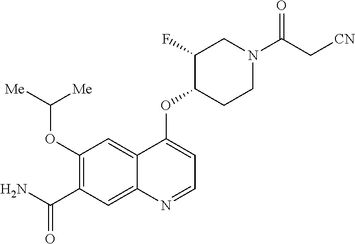 [SMILES: CC(C)Oc1cc2c(O[C@H]3CCN(C(=O)CC#N)C[C@H]3F)ccnc2cc1C(N)=O] | MS m/z 415 [M + H]⁺ LCMS Rt = 2.12 min (3R, 4S)-N-BOC-3-fluoro-4- hydroxypiperidine (PCT 2013116491). |

### Library Protocol 4

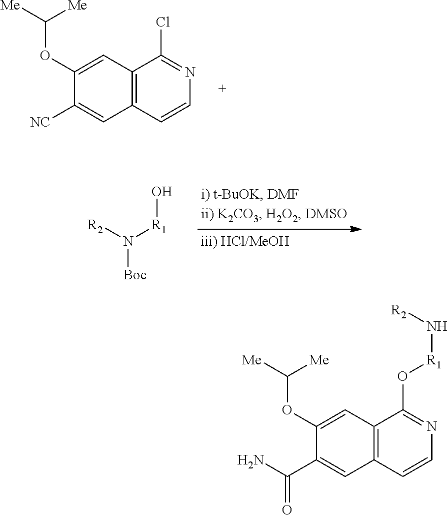

Step 1: To the appropriate N—BOC-aminoalcohol (162.5 μmol) was added a 0.125M solution of 1-chloro-7-isopropoxyisoquinoline-6-carbonitrile in DMSO (Preparation 35, 1000 μL, 125 μmol) followed by a 1M solution of t-BuOK in THF (162.5 μL, 162.5 μmol). The reaction vessels were capped and shaken at 55° C. for 1 h. The reactions were cooled and the reaction mixture used directly in the next step.

Step 2: To the reaction mixtures from Step 1 was added K₂CO₃ (70 mg, 500 μmol) followed by H₂O₂(30% in water, 200 μL). The reaction vessels were capped and shaken at 60° C. for 15 min. The reactions were quenched with saturated aq Na₂S₂O₃ solution (1 mL) at 0° C. The mixtures were concentrated in vacuo. The residues were diluted with MeOH (1 mL), filtered and concentrated in vacuo. The residues were used directly in the next step.

Step 3: To solutions of the residues from Step 2 in MeOH (500 μL) was added 4M HCl in dioxane (1 mL). The reaction vessels were capped and shaken at 30° C. for 2 h. The reaction mixtures were concentrated in vacuo and purified using preparative HPLC as described below.

Purification Method A: Phenomenex Gemini C18 250×21.2 mm, 8 μm; organic mobile phase: MeCN; aq mobile phase: ammonium hydroxide pH=10; Gradient time=8 or 9 min; flow rate=30 mL/min. Organic gradients were between 15-53%.

LCMS Method 1: XBRIDGE 50×2.1 mm, 5 μm; mobile phase A: 0.0375% TFA in water; mobile phase B: 0.01875% TFA in MeCN; gradient from 1% B to 5% B at 0.60 mins, further to 100% B at 4.00 mins and finally returning to 1% B at 4.30-4.70 mins; flow rate 0.8 mL/min.

Examples 19-21 were prepared according to the method described for Library Protocol 4 using of 1-chloro-7-isopropoxyisoquinoline-6-carbonitrile (Preparation 35) and the appropriate alcohol.

| Ex. | | | |
|---|---|---|---|
| No. | Name | Structure | Data/Starting Material |
| 19 | 1-{[(3R,4R)-3- fluoropiperidin-4- yl]oxy}-7-(propan-2- yloxy)isoquinoline-6- carboxamide | 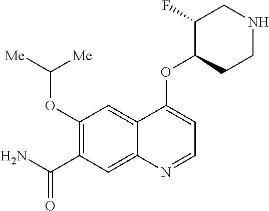 [SMILES: CC(C)Oc1cc2c(O[C@@H]3CCNC[C@H]3F)ccnc2cc1C(N)=O] | MS m/z 348 [M + H]⁺ LCMS Rt = 2.15 min (3R,4R)-1-BOC-3-fluoro-4- piperidinol. |
| 20 | 1-{[(3S.4R)-3- fluoropiperidin-4- yl]oxy}-7-(propan-2- yloxy)isoquinoline-6- carboxamide | 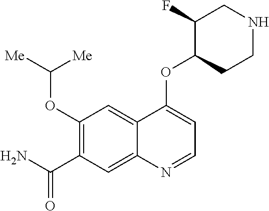 [SMILES: CC(C)Oc1cc2c(O[C@@H]3CCNC[C@@H]3F)ccnc2cc1C(N)=O] | MS m/z 348 [M + H]⁺ LCMS Rt = 2.21 min (3S,4R)-1-BOC-3-fluoro-4- piperidinol. |
| 21 | 1-[(2-oxopyrrolidin-3- yl)oxy]-7-(propan-2- yloxy)isoquinoline-6- carboxamide | 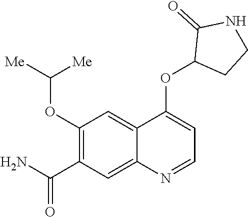 [SMILES: CC(C)Oc1cc2c(OC3CCNC3=O)ccnc2cc1C(N)=O] | MS m/z 330 [M + H]⁺ LCMS Rt = 2.37 min 3-hydroxy-2-pyrrolidinone using 4M tBuOK in THF at 30° C. for 16 h. |

### Library Protocol 5

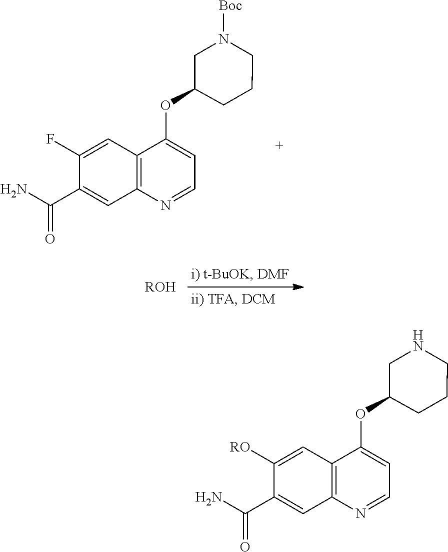

Step 1: To the desired alcohol (500 μmol) was added THF (400 μL) followed by a 1M solution of tBuOK in THF (450 μL, 450 μmol) and the reaction vessel was capped and shaken at 30° C. for 15 min. To the reaction mixture was added a 0.4M solution of (R)-tert-butyl 3-[(7-carbamoyl-6-fluoroquinolin-4-yl)oxy]piperidine-1-carboxylate in THF (Preparation 15, 250 μL, 100 μmol) and the reaction vessel was capped and shaken at 60° C. for 16 h. The reaction mixtures were concentrated in vacuo and purified using preparative HPLC as described below.

Step 2: The residues from Step 1 were dissolved in DCM (1.5 mL) and treated with TFA (150 μL). The reactions were capped and shaken at 30° C. for 30 min. The reaction mixtures were concentrated in vacuo to afford the desired compounds.

Purification Method A: Phenomenex Gemini C18 250×21.2 mm, 8 μm; organic mobile phase: MeCN; aq mobile phase: ammonium hydroxide pH=10; Gradient time=10 min; flow rate=30 mL/min. Organic gradient was between 42-72%.

Purification Method B: Phenomenex Gemini C18 250×21.2 mm, 8 μm; organic mobile phase: MeCN; aq mobile phase: 0.1% HCl; Gradient time=12 min; flow rate=30 mL/min. Organic gradient was between 36-66%.

LCMS Method: XBRIDGE 50×2.1 mm, 5 μm; mobile phase A: 0.05% NH₄OH in water; mobile phase B: 100% MeCN; gradient from 5% B to 100% B at 3.40 mins, hold at 100% B to 4.20 mins and finally returning to 5% B at 4.21-4.70 mins; flow rate 0.8 mL/min.

Examples 22 and 23 were prepared according to the method described for Library Protocol 5 using (R)-tert-butyl 3-[(7-carbamoyl-6-fluoroquinolin-4-yl)oxy]piperidine-1-carboxylate (Preparation 15) and the appropriate alcohol.

| Ex. | | | |
|---|---|---|---|
| No. | Name | Structure | Data/Starting Material |
| 22 | 6-methoxy-4-[(3R)- piperidin-3- yloxy]quinoline-7- carboxamide hydrochloride | 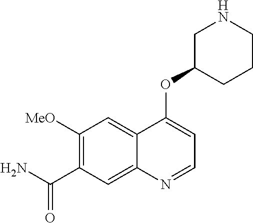 [SMILES: COc1cc2c(O[C@@H]3CCCNC3)ccnc2cc1C(N)=O] | MS m/z 302 [M + H]⁺ LCMS Rt = 2.01 min Methanol. Purification Method B. |
| 23 | 6-(cyclobutyloxy)-4- [(3R)-piperidin-3- yloxy]quinoline-7- carboxamide | 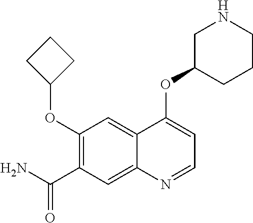 [SMILES: NC(=O)c1cc2nccc(O[C@@H]3CCCNC3)c2cc1OC1CCC1] | MS m/z 342 [M + H]⁺ LCMS Rt = 2.30 min Cyclobutanol. Purification Method A. |

### Library Protocol 6

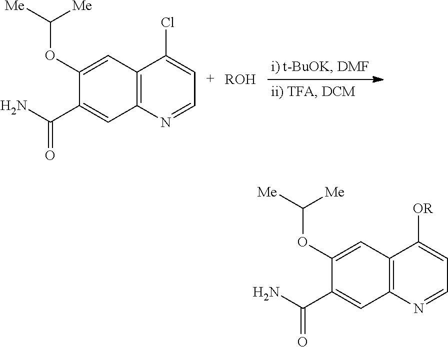

Step 1: To the appropriate N—BOC-amino alcohol (300 μmol) was added DMSO (0.5 mL) followed by a 2M solution of tBuOK in THF (100 μL, 200 μmol), and the reaction mixtures were stirred at 30° C. for 30 min. A 0.2M solution of 4-chloro-6-isopropoxyquinoline-7-carboxamide in DMSO (Preparation 38, 500 μL, 100 μmol) was added, and the reaction vessels were capped and shaken at 30° C. for 16 h. The reactions were concentrated in vacuo and purified using preparative HPLC as described below.

Step 2: To the residue from Step 1 was added a solution of TFA in DCM (2 mL, v:v 1:10), and the reaction vessels were capped and shaken at 30° C. for 1 h. The reaction mixtures were concentrated in vacuo to afford the desired compounds.

Purification Method A: Phenomenex Gemini C18 250×21.2 mm, 8 μm; organic mobile phase: MeCN; aq mobile phase: ammonium hydroxide pH=10; Gradient time=10 min; flow rate=30 mL/min. Organic gradient was between 38-72%.

Purification Method B: DIKMA Diamonsil C18 200×20 mm, 5 μm; organic mobile phase: MeCN; aq mobile phase: water with 0.225% formic acid. Gradient time=8 min; flow rate=35 mL/min. Organic gradient was between 18-48%.

LCMS Method: XBRIDGE 50×2.1 mm, 5 μm; mobile phase A: 0.05% NH₄OH in water; mobile phase B: 100% MeCN; gradient from 5% B to 100% B at 3.40 mins, hold at 100% B to 4.20 mins and finally returning to 5% B at 4.21-4.70 mins; flow rate 0.8 mL/min.

Examples 24-26 were prepared according to the method described for Library Protocol 6 using 4-chloro-6-isopropoxyquinoline-7-carboxamide (Preparation 38) and the appropriate alcohol.

| Ex. | | | |
|---|---|---|---|
| No. | Name | Structure | Data/Starting Material |
| 24 | 6-(propan-2-yloxy)-4- (pyrrolidin-3- yloxy)quinoline-7- carboxamide | 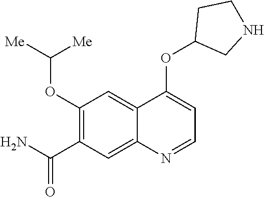 [SMILES: CC(C)Oc1cc2c(OC3CCNC3)ccnc2cc1C(N)=O] | MS m/z 316 [M + H]⁺; LCMS Rt = 2.12 min; N-BOC-3- hydroxypyrrolidine. Purification Method A. |
| 25 | 4-(piperidin-4-yloxy)-6- (propan-2- yloxy)quinoline-7- carboxamide | 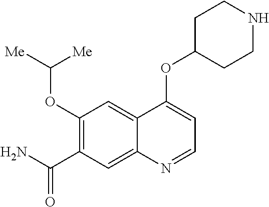 [SMILES: CC(C)Oc1cc2c(OC3CCNCC3)ccnc2cc1C(N)=O] | MS m/z 330 [M + H]⁺ LCMS Rt = 2.17 min N-BOC-4-hydroxypiperidine. Purification Method A. |
| 26 | 6-(propan-2-yloxy)-4- [(3S)-pyrrolidin-3- yloxy]quinoline-7- carboxamide formate | 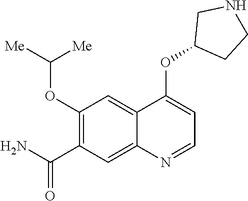 [SMILES: CC(C)Oc1cc2c(O[C@H]3CCNC3)ccnc2cc1C(N)=O] | MS m/z 316 [M + H]⁺ LCMS Rt = 2.12 min N-BOC-(S)-pyrrolidin-3-ol. Purification Method B. |

### Example 27

### 4-[(2-oxopyrrolidin-3-yl)oxy]-6-(propan-2-yloxy)quinoline-7-carboxamide

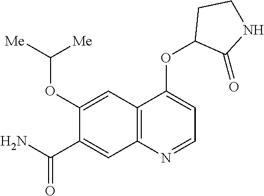 [SMILES: CC(C)Oc1cc2c(OC3CCNC3=O)ccnc2cc1C(N)=O]

To 3-hydroxypyrrolidine-2-one (100 μmol) in DMSO (500 μL) was added a 1M solution of tBuOK in THF (100 μL) and the reaction mixture was stirred at 30° C. for 30 min. A 0.1M solution of 4-chloro-6-isopropoxyquinoline-7-carboxamide in THF (Preparation 38, 500 μL, 50 μmol) was added and the reaction mixture was shaken at 30° C. for 16 h. The reaction was quenched by the addition of saturated ammonium chloride solution (50 μL, 100 μmol), filtered and purified using preparative HPLC as described below.

Purification Method: Phenomenex Gemini C18 250×21.2 mm, 8 μm; organic mobile phase: MeCN; aq mobile phase: ammonium hydroxide pH=10; Gradient time=10 min; flow rate=30 mL/min. Organic gradient was between 18-48%.

LCMS Method: XBRIDGE 50×2.1 mm, 5 μm; mobile phase A: 0.05% NH₄OH in water; mobile phase B: 100% MeCN; gradient from 5% B to 100% B at 3.40 mins, hold at 100% B to 4.20 mins and finally returning to 5% B at 4.21-4.70 mins; flow rate 0.8 mL/min. Rt=1.97 min MS m/z 330 [M+H]⁺

### Example 28

### 4-{[(3R)-1-(cyanoacetyl)piperidin-3-yl]oxy}-6-(propan-2-yloxy)quinoline-7-carboxamide

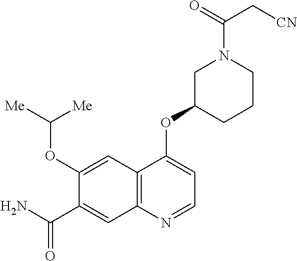 [SMILES: CC(C)Oc1cc2c(O[C@@H]3CCCN(C(=O)CC#N)C3)ccnc2cc1C(N)=O]

To (R)-4-(piperidin-3-yloxy)-6-(propan-2-yloxy)quinoline-7-carboxamide (Example 58, 500 mg, 1.37 mmol) in DCM was added 2-cyanoacetic acid (140 mg, 1.64 mmol), BOP (733 mg, 1.64 mmol) and TEA (0.57 mL, 4.10 mmol). The reaction mixture was stirred at room 0 temperature for 2 h before washing with water. The organic layer was dried over Na₂SO₄, filtered and concentrated in vacuo. The residue was triturated with DCM and diethyl ether to afford a yellow solid that was further purified by silica gel column chromatography eluting with 0-10% MeOH in DCM. The residue was triturated with DCM and diethyl ether to afford the title compound as a white solid (271 mg, 50%). ¹H NMR (400 MHz, DMSO-d₆): δ 8.62-8.60 (m, 1H), 8.22-8.20 (m, 1H), 7.70-7.50 (m, 2H), 7.50-7.40 (m, 1H), 7.20-7.00 (m, 1H), 5.00-4.80 (m, 2H), 4.30-3.60 (m, 5H), 3.40-3.10 (m, 3H), 2.10-1.70 (m, 2H), 1.60-1.40 (m, 6H).

LCMS Method 3: Thermo Hypersil Aquasil C18 50×2.1 mm, 5 μm; mobile phase A: 10 mM NH₄OAc in 95% water/5% MeCN; mobile phase B: 10 mM NH₄OAc in 95% MeCN/5% water; gradient of 0% B in 2.5 mins; flow rate 0.8 mL/min. MS m/z 397 [M+H]⁺

Examples 29-35 were prepared according to the method described for Example 28 using the appropriate piperidine as described below and 2-cyanoacetic acid unless otherwise described. Where purification differs from the original method it is noted in the table as such.

| Ex. | | | |
|---|---|---|---|
| No. | Name | Structure | Data/Starting Material |
| 29 | (R)-4-((1-(2- cyanoacetyl)piperidin-3- yl)oxy)-6-isopropoxy-2- methylquinoline-7- carboxamide | 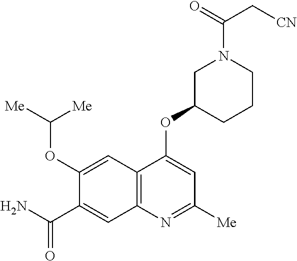 [SMILES: Cc1cc(O[C@@H]2CCCN(C(=O)CC#N)C2)c2cc(OC(C)C)c(C(N)=O)cc2n1] | Using (R)-6-isopropoxy-2-methyl-4- (piperidin-3-yloxy)quinoline-7- carboxamide (Example 68). The residue was purified using reverse phase column chromatography eluting with 35- 80% MeOH in water. |
| 30 | (R)-4-((1-(2- cyanoacetyl)piperidin-3- yl)oxy)-6- methoxyquinoline-7- carboxamide | 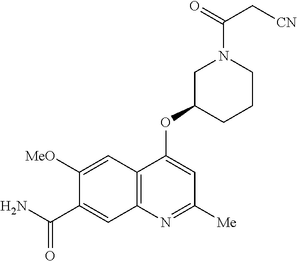 [SMILES: COc1cc2c(O[C@@H]3CCCN(C(=O)CC#N)C3)cc(C)nc2cc1C(N)=O] | Using (R)-6-methoxy-4-(piperidin-3- yloxy)quinoline-7-carboxamide (Example 58) |
| 31 | 4-{[(3R)-1- (cyanoacetyl)piperidin- 3-yl]oxy}-6- (difluoromethoxy) quinoline-7-carboxamide | 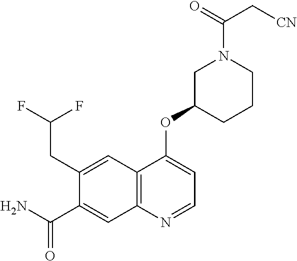 [SMILES: N#CCC(=O)N1CCC[C@@H](Oc2ccnc3cc(C(N)=O)c(CC(F)F)cc23)C1] | LCMS Rt = 1.17 min MS m/z 405 [M + H]⁺ Using (R)-6-(difluoromethoxy)-4- (piperidin-3-yloxy)quinoline-7- carboxamide (Example 67). Purified using preparative HPLC. |
| 32 | 4-{[(3R)-1- (cyanoacetyl)piperidin- 3-yl]oxy}-6- ethoxyquinoline-7- carboxamide | 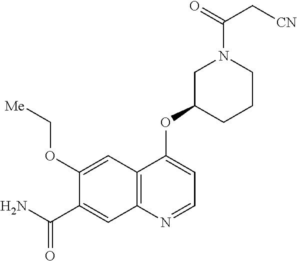 [SMILES: CCOc1cc2c(O[C@@H]3CCCN(C(=O)CC#N)C3)ccnc2cc1C(N)=O] | ¹H NMR (400 MHz, DMSO-d₆): δ 8.68 (m, 1H), 8.23-8.19 (m, 1H), 7.75 (br s, 1H), 7.70 (br s, 1H), 7.45-7.35 (m, 1H), 7.18-7.05 (m, 1H), 5.00-4.90 (br m, 1H), 4.30-4.10 (m, 4H), 3.80-3.50 (m, 2H), 2.10- 1.90 (m, 2H), 1.85-1.50 (m, 2H), 1.45-1.40 (m, 5H); MS m/z 384 [M + H]⁺; Using (R)-6-ethoxy-4- (piperidin-3-yloxy)quinoline-7- carboxamide (Example 65). |
| 33 | (R)-4-((1-(2- cyanoacetyl)piperidin-3- yl)oxy)-6- cyclobutoxyquinoline-7- carboxamide | 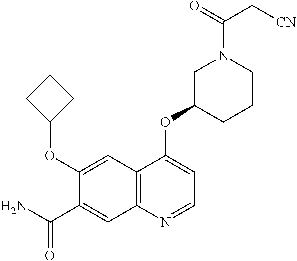 [SMILES: N#CCC(=O)N1CCC[C@@H](Oc2ccnc3cc(C(N)=O)c(OC4CCC4)cc23)C1] | Using (R)-6-cyclobutoxy-4- (piperidin-3-yloxy)quinoline-7- carboxamide (Example 23). |
| 34 | 4-{[(3R)-1-(2- cyanopropanoyl)piperidin- 3-yl]oxy}-6-(propan-2- yloxy)quinoline-7- carboxamide | 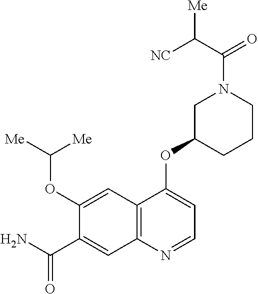 [SMILES: CC(C)Oc1cc2c(O[C@@H]3CCCN(C(=O)C(C)C#N)C3)ccnc2cc1C(N)=O] | ¹H NMR (400 MHz, DMSO-d₆): δ 8.60 (d, 1H), 8.26 (s, 1H), 7.50-7.30 (br m, 2H), 7.07-7.05 (m, 1H), 4.91- 4.81 (m, 2H), 4.40-4.20 (m, 1H), 4.10-3.60 (m, 2H), 3.45-3.35 (m, 1H), 2.07-1.95 (m, 2H), 1.90-1.50 (m, 2H), 1.64-1.38 (m, 10H). MS m/z 411 [M + H]⁺; Using (R)-4- (piperidin-3-yloxy)-6-(propan-2- yloxy)quinoline-7-carboxamide trifluoroacetate (Example 58) and racemic-2-cyanopropanoic acid. |
| 35 | 6-(difluoromethoxy)-4- [(3R)-piperidin-3- yloxy]quinoline-7- carboxamide | 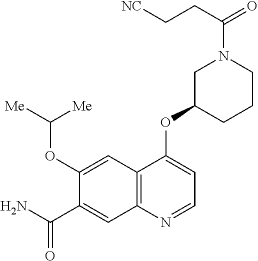 [SMILES: CC(C)Oc1cc2c(O[C@@H]3CCCN(C(=O)CCC#N)C3)ccnc2cc1C(N)=O] | ¹H NMR (400 MHz, DMSO-d₆): δ 8.65-8.60 (m, 1H), 8.22-8.20 (m, 1H), 7.80-7.60 (br s, 2H), 7.50-7.40 (m, 1H), 7.20-7.10 (m, 1H), 5.00 (br m, 1H), 4.90-4.75 (m, 2H), 4.10- 3.80 (m, 2H), 3.70-3.55 (m, 1H), 3.50-3.20 (m, 1H), 2.95-2.35 (m, 3H), 2.10-1.40 (m, 4H), 1.42-1.38 (m, 6H); MS m/z 411 [M + H]⁺ Using (R)-4-(piperidin-3-yloxy)-6- (propan-2-yloxy)quinoline-7- carboxamide trifluoroacetate (Example 58) and 3- cyanopropanoic add. |

### Example 36

### 1-{[(3R)-1-(cyanoacetyl)piperidin-3-yl]oxy}-7-(propan-2-yloxy)isoquinoline-6-carboxamide

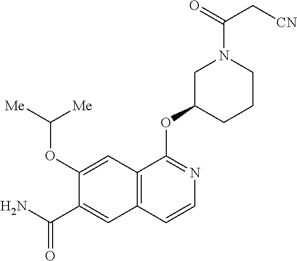 [SMILES: CC(C)Oc1cc2c(O[C@@H]3CCCN(C(=O)CC#N)C3)nccc2cc1C(N)=O]

To tert-butyl 3-{[6-carbamoyl-7-(propan-2-yloxy)isoquinolin-1-yl]oxy}piperidine-1-carboxylate (Preparation 3, 164 mg, 0.382 mmol) was added 4M HCl in dioxane and the reaction mixture was stirred at room temperature for 1 h. The reaction mixture was diluted with diethyl ether to enable precipitation of a solid. The precipitate was filtered and triturated with diethyl ether. The solid was dissolved in DMF and treated with TEA (0.213 mL, 1.53 mmol) followed by BOP (256 mg, 0.573 mmol) and 2-cyanoacetic acid (36 mg, 0.420 mmol). The reaction mixture was stirred at room temperature overnight. The reaction mixture was diluted with EtOAc (20 mL) and washed with water. The aq layer was extracted with EtOAc (15 mL), the organic layers were combined, washed with water (2×30 mL), brine (30 mL), dried over Na₂SO₄, filtered and concentrated in vacuo. The residue was purified using silica gel column chromatography eluting with 50-100% EtOAc in hexanes to afford the title compound as a white solid (132 mg, 87%). ¹H NMR (400 MHz, DMSO-d₆): b ppm 9.40-9.30 (br s, 1H), 9.10-9.00 (br s, 1H), 8.20 (s, 1H), 7.90-7.85 (m, 1H), 7.85-7.80 (m, 1H), 7.80-7.70 (br s, 1H), 7.45 (d, 1H), 5.00-5.05 (m, 1H), 3.45-3.40 (m, 2H), 3.30-3.20 (m, 1H), 3.15-3.05 (m, 1H), 2.00-1.90 (m, 3H), 1.80-1.70 (m, 1H), 1.40-1.35 (m, 6H). MS m/z 397 [M+H]⁺

### Example 37

### 4-chloro-1-{[(3R)-1-(cyanoacetyl)piperidin-3-yl]oxy}-7-(propan-2-yloxy)isoquinoline-6-carboxamide

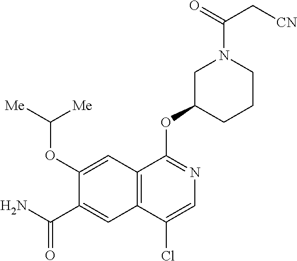 [SMILES: CC(C)Oc1cc2c(O[C@@H]3CCCN(C(=O)CC#N)C3)ncc(Cl)c2cc1C(N)=O]

The title compound was prepared as described for Example 36 using (R)-tert-butyl 3-{[6-carbamoyl-4-chloro-7-(propan-2-yloxy)isoquinolin-1-yl]oxy}piperidine-1-carboxylate (Preparation 14) and was purified using preparative HPLC. LCMS Method 3: Rt=1.59 min. MS m/z 431 [M+H]⁺

### Example 38

### 4-cyano-1-{[(3R)-1-(cyanoacetyl)piperidin-3-yl]oxy}-7-(propan-2-yloxy)isoquinoline-6-carboxamide

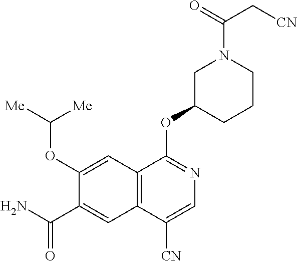 [SMILES: CC(C)Oc1cc2c(O[C@@H]3CCCN(C(=O)CC#N)C3)ncc(C#N)c2cc1C(N)=O]

The title compound was prepared as described for Example 36 using (R)-tert-butyl 3-{[6-carbamoyl-4-cyano-7-(propan-2-yloxy)isoquinolin-1-yl]oxy}piperidine-1-carboxylate (Preparation 2) and was purified by preparative HPLC. ¹H NMR (400 MHz, DMSO-d₆): δ 8.60-8.55 (m, 1H), 8.20-8.15 (m, 1H), 7.80 (br s, 1H), 7.75 (br s, 1H), 7.60-7.55 (m, 1H), 5.50-5.45 (m, 1H), 4.95-4.80 (m, 1H), 4.40-3.60 (m, 5H), 2.20-1.50 (m, 5H), 1.42-1.38 (m, 6H). MS m/z 422 [M+H]⁺

### Example 39

### 4-{[(3S)-1-(cyanoacetyl)pyrrolidin-3-yl]oxy}-6-(propan-2-yloxy)quinoline-7-carboxamide

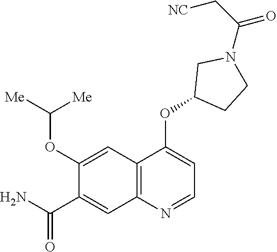 [SMILES: CC(C)Oc1cc2c(O[C@H]3CCN(C(=O)CC#N)C3)ccnc2cc1C(N)=O]

To a solution of (S)-tert-butyl 3-{[7-carbamoyl-6-(propan-2-yloxy)quinolin-4-yl]oxy}pyrrolidine-1-carboxylate (Preparation 10, 200 mg, 0.52 mmol) in DCM (5 mL) was added TFA (8 mL) dropwise at 0° C. The reaction mixture was stirred at room temperature for 12 h before concentrating in vacuo. The residue was triturated with diethyl ether and EtOAc and was then dissolved in DMF (1.5 mL) and treated with TEA (0.2 mL, 1.22 mmol). The reaction mixture was stirred for 10 min. BOP (427 mg, 0.965 mmol) was added followed by 2-cyanoacetic acid (47 mg, 0.552 mmol) and the reaction mixture was stirred at room temperature for 2 h. The reaction mixture was diluted with water and extracted into EtOAc. The organic layer was collected, washed with brine, dried over Na₂SO₄ and concentrated in vacuo. The residue was purified using silica gel column chromatography to afford the title compound (30 mg, 30% over 2 steps). ¹H NMR (400 MHz, DMSO-d₆): δ 8.66-8.63 (m, 1H), 8.23 (s, 1H), 7.71 (br s, 1H), 7.49-7.47 (m, 1H), 7.10-7.07 (m, 1H), 5.50-5.30 (m, 1H), 4.90-4.80 (m, 1H), 4.00-3.60 (m, 5H), 2.40-2.20 (m, 3H), 1.40-1.35 (m, 6H). MS m/z 383 [M+H]⁺

### Example 40

### 4-{[(3R)-1-(cyanoacetyl)pyrrolidin-3-yl]oxy}-6-(propan-2-yloxy)quinoline-7-carboxamide

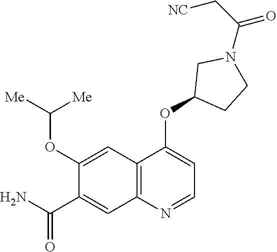 [SMILES: CC(C)Oc1cc2c(O[C@@H]3CCN(C(=O)CC#N)C3)ccnc2cc1C(N)=O]

The title compound was prepared according to the method described for Example 39 using (R)-tert-butyl 3-{[7-carbamoyl-6-(propan-2-yloxy)quinolin-4-yl]oxy}pyrrolidine-1-carboxylate (Preparation 11). ¹H NMR (400 MHz, DMSO-d₆): δ 8.66-8.63 (m, 1H), 8.23 (s, 1H), 7.71 (br s, 1H), 7.49-7.47 (m, 1H), 7.10-7.07 (m, 1H), 5.50-5.30 (m, 1H), 4.90-4.80 (m, 1H), 4.00-3.60 (m, 5H), 2.40-2.20 (m, 3H), 1.40-1.35 (m, 6H). MS m/z 383 [M+H]⁺

### Example 41

### 1-{f[1-(2-hydroxypropyl)piperidin-4-yl]oxy}-7-(propan-2-yloxy)isoquinoline-6-carboxamide

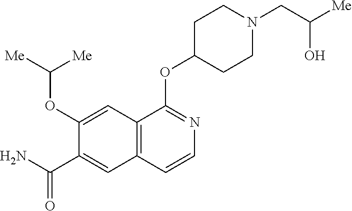 [SMILES: CC(O)CN1CCC(Oc2nccc3cc(C(N)=O)c(OC(C)C)cc23)CC1]

To a solution of 1-(piperidin-4-yloxy)-7-(propan-2-yloxy)isoquinoline-6-carboxamide (Example 60, 60 mg, 0.18 mmol) in DMF (1.82 mL) was added NaH (8 mg, 0.20 mmol) and 1-bromopropan-2-ol (28 mg, 0.20 mmol) and the reaction mixture was heated to 60° C. until complete. The reaction mixture was cooled, extracted into EtOAc, washed with brine, dried over magnesium sulfate and concentrated in vacuo. The residue was purified using preparative HPLC. LCMS Method 3: Rt=2.87 min; MS m/z 388 [M+H]⁺

### Example 42

### 4-{[(3R)-1-(hydroxyacetyl)piperidin-3-yl]oxy}-2-methyl-6-(propan-2-yloxy)quinoline-7-carboxamide

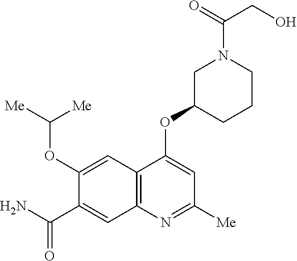 [SMILES: Cc1cc(O[C@@H]2CCCN(C(=O)CO)C2)c2cc(OC(C)C)c(C(N)=O)cc2n1]

A solution of (R)-6-isopropoxy-2-methyl-4-(piperidin-3-yloxyquinoline-7-carboxamide (Example 68, 39 mg, 0.10 mmol), 2-(benzyloxy)acetyl chloride (23 mg, 0.124 mmol) and TEA (0.022 mL, 0.154 mmol) in DCM was stirred at room temperature for 30 min. The reaction mixture was concentrated in vacuo and partitioned between EtOAc (10 mL) and water (10 mL). The organic layer was washed with brine, dried over Na₂SO₄ and concentrated in vacuo. The residue was dissolved in MeOH and treated with palladium hydroxide on carbon. Hydrogen gas was bubbled through the mixture and the reaction mixture was then stirred under hydrogen atmosphere for 2 h. The reaction mixture was filtered through Celite, concentrated in vacuo and purified using reverse phase column chromatography eluting with 35-90% MeOH in water containing 0.1% formic acid.

### Example 43

### 4-[(1-acetylpiperidin-3-yl)oxy]-6-(propan-2-yloxy)quinoline-7-carboxamide

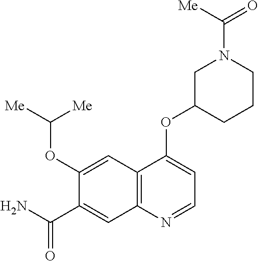 [SMILES: CC(=O)N1CCCC(Oc2ccnc3cc(C(N)=O)c(OC(C)C)cc23)C1]

The title compound was prepared according to the methods described for Example 58 Step 1 using 4-chloro-6-isopropoxyquinoline-7-carboxamide (Preparation 38) and 1-(3-hydroxypiperidin-1-yl)ethan-1-one. ¹H NMR (400 MHz, DMSO-d₆): δ 8.65-8.60 (m, 1H), 8.40-8.30 (m, 1H), 7.60 (br s, 2H), 7.50-7.40 (m, 1H), 7.17-7.07 (m, 1H), 5.00-4.70 (m, 2H), 4.20-3.90 (m, 2H), 3.70-3.00 (m, 4H), 2.00-1.30 (m, 11H). MS m/z 372 [M+H]⁺

### Example 44

### 1-[(1-acetylpiperidin-4-yl)oxy]-7-(propan-2-yloxy)isoquinoline-6-carboxamide

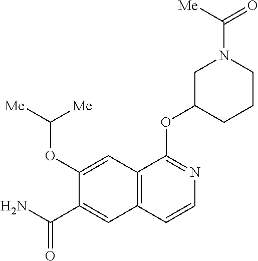 [SMILES: CC(=O)N1CCCC(Oc2nccc3cc(C(N)=O)c(OC(C)C)cc23)C1]

The title compound was prepared according to the methods described for Example 58 Step 1 using 1-chloro-7-isopropoxyisoquinoline-6-carboxamide (Preparation 37) and 1-(3-hydroxypiperidin-1-yl)ethan-1-one. The product was purified by preparative HPLC. LCMS Method 3: Rt=3.47 min MS m/z 372 [M+H]⁺

### Example 45

### 4-{[1-(methylsulfonyl)piperidin-3-yl]oxy}-6-(propan-2-yloxy)quinoline-7-carboxamide

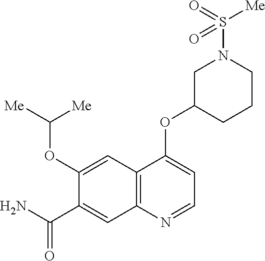 [SMILES: CC(C)Oc1cc2c(OC3CCCN(S(C)(=O)=O)C3)ccnc2cc1C(N)=O]

The title compound was prepared according to the methods described for Example 58 Step 1 using 4-chloro-6-isopropoxyquinoline-7-carboxamide (Preparation 38) and 1-(methylsulfonyl)piperidin-3-ol. The product was purified by reverse phase column chromatography eluting with 5-95% MeCN in water modified with TEA. ¹H NMR (400 MHz, DMSO-d₆): δ 8.65-8.63 (m, 1H), 8.22 (s, 1H), 7.75-7.65 (br m, 2H), 7.58 (s, 1H), 7.15-7.10 (m, 1H), 4.95-4.90 (m, 1H), 4.85-4.78 (m, 1H), 3.55-3.45 (m, 2H), 3.38-3.28 (m, 1H), 3.20-3.15 (m, 1H), 2.90 (s, 3H), 2.00-1.85 (m, 3H), 1.70-1.60 (m, 1H), 1.40-1.38 (m, 6H). MS m/z 408 [M+H]⁺

### Example 46

### 4-({(3R)-1-[(2S)-2-amino-3-cyanopropanoyl]piperidin-3-yl}oxy)-6-(propan-2-yloxy)quinoline-7-carboxamide trifluoroacetate

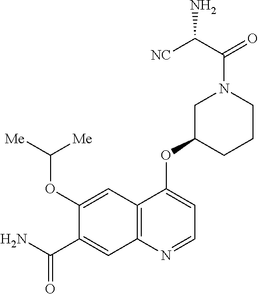 [SMILES: CC(C)Oc1cc2c(O[C@@H]3CCCN(C(=O)[C@@H](N)C#N)C3)ccnc2cc1C(N)=O]

Step 1: To a solution of (R)-4-(piperidin-3-yloxy)-6-(propan-2-yloxy)quinoline-7-carboxamide trifluoroacetate (Example 58, 200 mg, 0.452 mmol) in DMF (2 mL) was added TEA (0.2 mL) at 0° C. The reaction was stirred for 30 min before the addition of BOP (300 mg, 0.678 mmol) and (S)-2-((tert-butoxycarbonyl)amino)-3-cyanopropanoic acid (87 mg, 0.406 mmol), and the reaction mixture was stirred at room temperature for 2 h. The reaction mixture was diluted with water and extracted into EtOAc. The organic layer was washed with brine, dried over Na₂SO₄ and concentrated in vacuo. The residue was purified using preparative TLC.

Step 2: The residue from Step 1 was dissolved in DCM (2 mL). The solution was treated with TFA (0.6 mL) dropwise at 0° C. and the reaction mixture was stirred at room temperature for 2 h. The reaction mixture was concentrated in vacuo and the resulting solid was triturated with diethyl ether to afford the title compound (30 mg, 17% over two steps). ¹H NMR (400 MHz, DMSO-d₆): δ 8.65-8.60 (m, 1H), 8.25-8.20 (m, 1H), 7.80-7.70 (m, 2H), 7.50-7.45 (m, 1H), 7.15-7.05 (m, 1H), 5.00-4.75 (m, 2H), 4.10-3.50 (m, 4H), 3.00-2.50 (m, 3H), 2.10-1.50 (m, 4H), 1.40-1.30 (m, 6H). MS m/z 426 [M+H]⁺

Examples 47 and 48 were prepared according to the method described for Example 46 using the appropriate piperidine and carboxylic acid as described below. Where purification or methods differ from the original method, it is noted in the table as such.

| Ex. | | | |
|---|---|---|---|
| No. | Name | Structure | Data/Starting Material |
| 47 | 1-{[1-(D-alanyl)piperidin- 4-yl]oxy}-7-(propan-2- yloxy)isoquinoline-6- carboxamide trifluoroacetate | 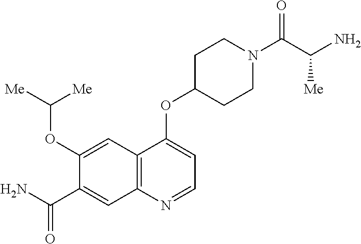 [SMILES: CC(C)Oc1cc2c(OC3CCN(C(=O)[C@@H](C)N)CC3)ccnc2cc1C(N)=O] | LCMS Method 3: Rt = 2.66 min MS m/z 401 [M + H]⁺ Using 1-(piperidin-4-yloxy)-7- (propan-2-yloxy)isoquinoline-6- carboxamide (Example 60) and tert-butyl (D-alanyl)carbamate. Purification by preparative HPLC. |
| 48 | 4-({(3R)-1-[(2R)-2-amino- 3- cyanopropanoyl]piperidin- 3-yl}oxy)-6-(propan-2- yloxy)quinoline-7- carboxamide trifluoroacetate | 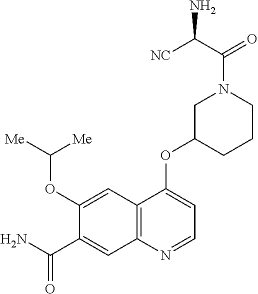 [SMILES: CC(C)Oc1cc2c(OC3CCCN(C(=O)[C@H](N)C#N)C3)ccnc2cc1C(N)=O] | ¹H NMR (400 MHz, DMSO-d₆): δ 8.65-8.60 (m, 1H), 8.25-8.20 (m, 1H), 7.70-7.60 (m, 2H), 7.50-7.45 (m, 1H), 7.20-7.05 (m, 1H), 5.00- 4.75 (m, 2H), 4.10-3.50 (m, 4H), 2.90-2.50 (m, 3H), 2.20-1.50 (m, 4H), 1.40-1.30 (m, 6H); MS m/z 426 [M + H]⁺; Using (R)-2-((tert- butoxycarbonyl)amino)-3- cyanopropanoic acid and (R)-4- (piperidin-3-yloxy)-6-(propan-2- yloxy)quinoline-7-carboxamide (Example 58). |

### Example 49

### 1-{[(3R,4S)-1-(cyanoacetyl)-4-methylpiperidin-3-yl]oxy}-7-(propan-2-yloxy)isoquinoline-6-carboxamide

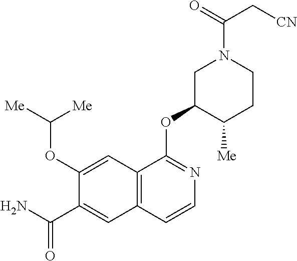 [SMILES: CC(C)Oc1cc2c(O[C@H]3CN(C(=O)CC#N)CC[C@@H]3C)nccc2cc1C(N)=O]

Step 1: To a solution of 1-chloro-7-isopropoxyisoquinoline-6-carbonitrile (Preparation 37, 247 mg, 1.04 mmol) in DMSO (10 mL) was added tert-butyl (3R,4S)-3-hydroxy-4-methylpiperidine-1-carboxylate (may be prepared as described for the diastereomer in patent US 2011 0092698, 258 mg, 1.20 mmol) and tBuOK (145 mg, 1.25 mmol). The reaction mixture was heated to 60° C. until complete. Step 2: The reaction mixture was cooled and treated with K₂CO₃ (3.1 mmol) and H₂O₂(0.350 mL). The reaction mixture was heated at 60° C. for 30 min and was then cooled to room temperature and poured into water. The resultant white precipitate was filtered, washed with water, dried and dissolved in the minimum amount of dioxane. 4M HCl in dioxane (1 mL) was added and the reaction mixture was allowed to stand at room temperature for 1 h. The reaction mixture was concentrated in vacuo and the resulting solid was washed with MeCN followed by diethyl ether and then dried. Step 3:The solid was combined with 2-cyanoacetic acid (46 mg, 0.542 mmol), TEA (0.19 mL, 1.36 mmol) and BOP (242 mg, 0.542 mmol) in DCM and the resulting mixture was stirred at room temperature for 2 h. The reaction mixture was treated with 1M aq NaOH and extracted with DCM. The organic layer was dried over Na₂SO₄, filtered and concentrated in vacuo. The residue was purified using silica gel column chromatography eluting with 10% MeOH in DCM to afford the title compound (60 mg, 32%).

Examples 50-52 were prepared according to the method described for Example 49 using the appropriate isoquinoline, hydroxypiperidine and carboxylic acid as described below. Where purification or methods differ from the original method, it is noted in the table as such.

| Ex. | | | |
|---|---|---|---|
| No. | Name | Structure | Data/Starting Material |
| 50 | 1-{[1- (cyanoacetyl)piperidin- 4-yl]oxy}-7- (propan-2- yloxy)isoquinoline-6- carboxamide | 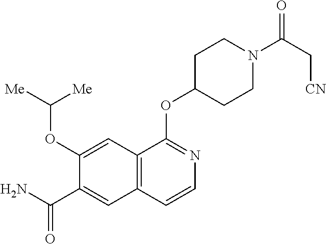 [SMILES: CC(C)Oc1cc2c(OC3CCN(C(=O)CC#N)CC3)nccc2cc1C(N)=O] | ¹H NMR (400 MHz, DMSO-d₆): δ 8.20 (s, 1H), 7.90-7.87 (m, 1H), 7.70 (br s, 2H), 7.60 (s, 1H), 7.40 (d. 1H), 5.55- 5.45 (m, 1H), 4.90-4.75 (m, 1H), 4.10 (s, 2H), 3.80-3.40 (m, 4H), 2.20-1.70 (m, 4H), 1.40 (d, 6H); MS m/z 397 [M + H]⁺; Using 1-chloro-7- isopropoxyisoquinoline-6-carbonitrile (Preparation 37), N-BOC-4- hydroxypiperidine and 2-cyanoacetic acid. |
| 51 | 1-{[1- (hydroxyacetyl)piperidin- 4-yl]oxy}-7- (propan-2- yloxy)isoquinoline-6- carboxamide | 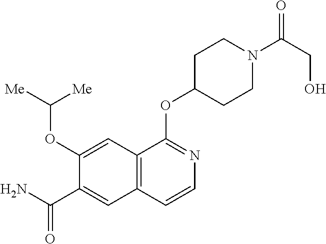 [SMILES: CC(C)Oc1cc2c(OC3CCN(C(=O)CO)CC3)nccc2cc1C(N)=O] | LCMS Rt = 3.48 min MS m/z 388 [M + H]⁺; Using 1-chloro-7- isopropoxyisoquinoline-6-carbonitrile (Preparation 37), N-BOC-4- hydroxypiperidine and glycolic acid. Purification using preparative HPLC. |
| 52 | 4-{[(3R,4S)-1- (cyanoacetyl)-4- methylpiperidin-3- yl]oxy}-6-(propan-2- yloxy)quinoline-7- carboxamide | 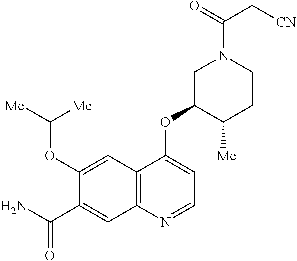 [SMILES: CC(C)Oc1cc2c(O[C@H]3CN(C(=O)CC#N)CC[C@@H]3C)ccnc2cc1C(N)=O] | ¹H NMR (400 MHz, DMSO-d₆): δ 8.65-8.63 (m, 1H), 8.23 (s, 1H), 7.70 (br s, 2H), 7.52-7.48 (m, 1H), 7.25- 7.23 (m, 0.5H), 7.05-7.02 (m, 0.5H), 4.90-4.80 (m, 1H), 4.55-3.88 (m, 4.5H), 3.65-3.55 (m, 0.5H), 3.35-3.25 (m, 2.5H), 3.15-3.05 (m, 0.5H), 1.45- 1.40 (m, 8H), 1.10-1.05 (m, 3H); using 4-chloro-6-isopropoxyquinoline-7- carboxamide (Preparation 38) and tert-butyl (3R,4S)-3-hydroxy-4- methylpiperidine-1-carboxylate¹ and 2-cyanoacetic acid. |

¹may be prepared as described for the diastereomer in patent US 2011 0092698

### Example 53

### 1-[(3R)-piperidin-3-yloxy]-7-(propan-2-yloxy)isoquinoline-6-carboxamide hydrochloride

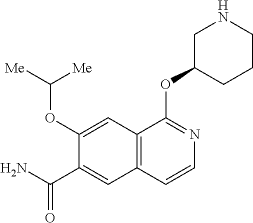 [SMILES: CC(C)Oc1cc2c(O[C@@H]3CCCNC3)nccc2cc1C(N)=O]

To (R)-tert-butyl 3-{[6-carbamoyl-7-(propan-2-yloxy)isoquinolin-1-yl]oxy}piperidine-1-carboxylate (Preparation 3, 147 mg, 0.342 mmol) was added 4M HCl in dioxane (1.71 mL) and the reaction mixture was stirred at room temperature for 1 h. The reaction mixture was diluted with diethyl ether and the resultant precipitate was collected by filtration. The solid was washed with diethyl ether to afford the title compound as the hydrochloride salt (127 mg, 92%). ¹H NMR (400 MHz, DMSO-d₆): δ 9.40 (br s, 1H), 9.05 (br s, 1H), 7.90-7.80 (m, 1H), 7.70 (br s, 2H), 7.50 (d, 1H), 5.00 (m, 1H), 3.50-3.35 (m, 3H), 3.20-3.00 (m, 2H), 2.00-1.90 (m, 3H), 1.80-1.70 (m, 1H), 1.40-1.30 (m, 6H). MS m/z 330 [M+H]⁺

Examples 54-57 were prepared according to the method described for Example 58 using the appropriate BOC-protected piperidine as described below. The Examples were isolated as the hydrochloride salts, or where purification or methods differ from the original method, it is noted in the table as such.

| Ex. | | | |
|---|---|---|---|
| No. | Name | Structure | Data/Starting Material |
| 54 | 4-bromo-1-[(3R)- piperidin-3-yloxy]-7- (propan-2- yloxy)isoquinoline-6- carboxamide hydrochloride | 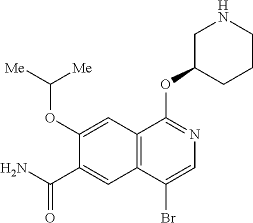 [SMILES: CC(C)Oc1cc2c(O[C@@H]3CCCNC3)ncc(Br)c2cc1C(N)=O] | ¹H NMR (400 MHz, DMSO-d₆): δ 8.25 (s, 1H), 8.15 (s, 1H), 7.85 (br s, 1H), 7.70 (br s, 1H), 7.68 (s, 1H), 5.15-5.05 (m, 1H), 3.18-3.12 (m, 1H), 2.80-2.50 (m, 4H), 2.15-2.05 (m, 1H), 1.75-1.60 (m, 2H), 1.50-1.45 (m, 1H), 1.40-1.38 (m, 6H); MS m/z 410 [M⁸¹Br + H]⁺ Using (R)-tert-butyl 3-{[4-bromo-6- carbamoyl-7-(propan-2- yloxy)isoquinolin-1-yl]oxy}piperidine-1- carboxylate (Preparation 5). |
| 55 | 4-chloro-1-[(3R)- piperidin-3-yloxy]-7- (propan-2- yloxy)isoquinoline-6- carboxamide hydrochloride | 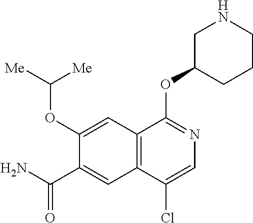 [SMILES: CC(C)Oc1cc2c(O[C@@H]3CCCNC3)ncc(Cl)c2cc1C(N)=O] | ¹H NMR (400 MHz, DMSO-d₆): δ 8.30 (s, 1H), 8.05 (s, 1H), 7.85 (br s, 1H), 7.60 (br s, 1H), 7.68 (s, 1H), 5.15-5.10 (m, 1H), 3.18-3.12 (m, 1H), 2.80-2.50 (m, 4H), 2.15-2.05 (m, 1H), 1.78-1.68 (m, 2H), 1.55-1.45 (m, 1H), 1.42-1.40 (m, 6H); MS m/z 364 [M + H]⁺ Using (R)-tert-butyl 3-{[6-carbamoyl-4- chloro-7-(propan-2-yloxy)isoquinolin-1- yl]oxy}piperidine-1-carboxylate (Preparation 6). |
| 56 | 4-methyl-1-[(3R)- piperidin-3-yloxy]-7- (propan-2- yloxy)isoquinoline-6- carboxamide hydrochloride | 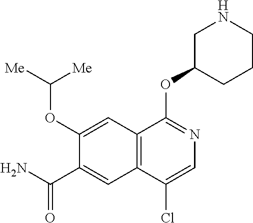 [SMILES: CC(C)Oc1cc2c(O[C@@H]3CCCNC3)ncc(Cl)c2cc1C(N)=O] | ¹H NMR (400 MHz, DMSO-d₆): δ 8.20 (s, 1H), 7.80-7.65 (m, 3H), 7.60 (s, 1H), 5.15-5.05 (m, 1H), 3.20-3.15 (m, 1H), 2.80-2.65 (m, 2H), 2.62-2.50 (m, 2H), 2.45 (s, 3H), 2.10-2.05 (m, 1H), 1.78-1.65 (m, 2H), 1.55-1.45 (m, 1H), 1.40-1.35 (m, 6H); MS m/z 344 [M + H]⁺ Using (R)-tert-butyl 3-{[6-carbamoyl-4- methyl-7-(propan-2-yloxy)isoquinolin- 1-yl]oxy}piperidine-1-carboxylate (Preparation 4). |
| 57 | 3-isopropoxy-5- (piperidin-3-yloxy)-2- naphthamide hydrochloride | 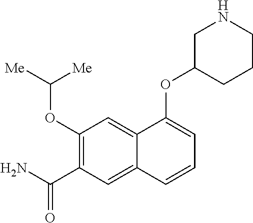 [SMILES: CC(C)Oc1cc2c(OC3CCCNC3)cccc2cc1C(N)=O] | ¹H NMR (400 MHz, DMSO-d₆): δ 9.15 (br s, 1H), 8.85 (s, 1H), 8.20-8.15 (m, 2H), 8.10-8.05 (m, 1H), 7.60-7.55 (m, 1H), 7.65-7.60 (m, 1H), 5.45-5.35 (m, 1H), 3.90-3.60 (m, 5H), 2.75-2.45 (m, 4H), 1.93 (d, 6H); tert-butyl 3-((6- carbamoyl-7-isopropoxynaphthalen-1- yl)oxy)piperidine-1 -carboxylate (Preparation 27). |

### Example 58

### (R)-4-(piperidin-3-yloxy)-6-(propan-2-yloxy)quinoline-7-carboxamide hydrochloride

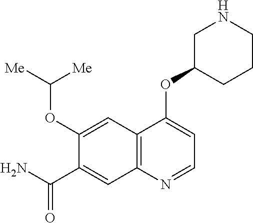 [SMILES: CC(C)Oc1cc2c(O[C@@H]3CCCNC3)ccnc2cc1C(N)=O]

Step 1: To a solution of 4-chloro-6-isopropoxyquinoline-7-carboxamide (Preparation 38, 400 mg, 1.51 mmol) in DMSO (15 mL) was added (R)-1-N—BOC-2-hydroxypiperidine (384 mg, 1.81 mmol) followed by tBuOK (210 mg, 1.81 mmol) and the reaction mixture was heated to 60° C. for 3 h. The reaction mixture was diluted with water (150 mL) and extracted into EtOAc (3×60 mL). The organic layers were collected, washed with brine (30 mL), dried over Na₂SO₄, filtered and concentrated in vacuo. The residue was triturated with diethyl ether and hexanes to afford a white solid.

Step 2: The intermediate residue from Step 1 was dissolved in dioxane (4 mL) and treated with 4M HCl in dioxane (3 mL). The reaction mixture was left to stand for 1 h before concentrating in vacuo. The residue was triturated with diethyl ether to afford the title compound as the hydrochloride salt as a yellow solid (490 mg, 89%).

The trifluoroacetate salt of the title compound may be prepared as follows: The intermediate residue from Step 1 was dissolved in DCM (20 mL) and treated with TFA (10 mL) dropwise at 0° C. The reaction mixture was stirred at room temperature for 12 h then concentrated in vacuo. The residue was triturated with diethyl ether to afford the title compound as the trifluoroacetate salt as a solid (90% yield). ¹H NMR (400 MHz, DMSO-d₆): δ 9.10-8.70 (s, 3H), 8.30 (d, 1H), 7.90 (s, 1H), 7.80 (d, 1H), 7.40 (s, 1H), 5.20 (m, 1H), 4.90 (m, 1H), 3.60-3.10 (m, 4H), 2.20-1.80 (m, 4H), 1.40 (m, 6H).

Examples 59-63 were prepared according to the method described for Example 58 using the appropriate heteroaryl chlorides as described below. The Examples were isolated as the hydrochloride or TFA salts, or where purification or methods differ from the original method, it is noted in the table as such.

| Ex. | | | |
|---|---|---|---|
| No. | Name | Structure | Data/Starting Material |
| 59 | 4-{[(3R,6S)-6- methylpiperidin-3- yl]oxy}-6-(propan-2- yloxy)quinoline-7- carboxamide | 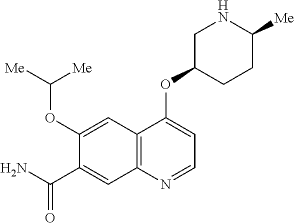 [SMILES: CC(C)Oc1cc2c(O[C@@H]3CC[C@H](C)NC3)ccnc2cc1C(N)=O] | ¹H NMR (400 MHz, DMSO-d₆): δ 8.60 (d, 1H), 8.20 (s, 1H), 7.75-7.65 (br m, 2H), 7.50 (s, 1H), 7.10 (d, 1H), 4.85- 4.80 (m, 1H), 4.60-4.55 (m, 1H), 3.35- 3.30 (m, 1H), 2.70-2.60 (m, 2H), 2.30- 2.20 (m, 1H), 1.80-1.72 (m, 1H), 1.65- 1.55 (m, 1H), 1.40-1.38 (m, 6H), 1.36- 1.23 (m, 1H), 1.05 (d, 3H); MS m/z 344 [M + H]⁺ Using 4-chloro-6-isopropoxyquinoline-7- carboxamide (Preparation 38) and tert- butyl (2S, 5R)-5-hydroxy-2- methylpiperidine-1-carboxylate. |
| 60 | 1-(piperidin-4-yloxy)- 7-(propan-2- yloxy)isoquinoline-6- carboxamide | 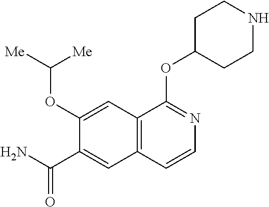 [SMILES: CC(C)Oc1cc2c(OC3CCNCC3)nccc2cc1C(N)=O] | ¹H NMR (400 MHz, DMSO-d₆): δ 8.70- 8.55 (br s, 2H), 8.20 (s, 1H), 7.90 (d, 1H), 7.75 (br s, 2H), 7.60 (s, 1H), 7.45 (d, 1H), 5.55-5.48 (m, 1H), 4.95-4.90 (m, 1H), 3.40-3.15 (m, 4H), 2.25-2.17 (m, 2H), 2.08-1.98 (m, 1H), 1.40 (d, 1H). MS m/z 330 [M + H]⁺; 1-chloro-7- isopropoxyisoquinoline-6-carboxamide (Preparation 37) and N-BOC-4- hydroxypiperidine. Isolated as the TFA salt. |
| 61 | 4-{[(3S,4R)-3- hydroxypiperidin-4- yl]oxy}-6-(propan-2- yloxy)quinoline-7- carboxamide | 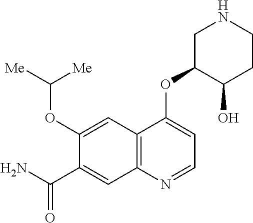 [SMILES: CC(C)Oc1cc2c(O[C@H]3CNCC[C@H]3O)ccnc2cc1C(N)=O] | ¹H NMR (400 MHz, DMSO-d₆): δ 8.90- 8.85 (m, 1H), 8.35 (s, 1H), 7.75 (s, 1H), 7.50-7.45 (m, 1H), 5.25-5.10 (m, 1H), 5.05-4.95 (m, 1H), 4.30-4.28 (m, 1H), 3.35-3.10 (m, 4H). 2.30-2.20 (m, 1H), 2.15-2.05 (m, 1H), 1.45-1.35 (m, 6H). MS m/z 346 [M + H]⁺; Step 1 only using 4-chloro-6-isopropoxyquinoline-7- carboxamide (Preparation 38) and tert- butyl (3S,4R)-3,4-dihydroxypiperidine-1- carboxylate (J. Org. Chem. 2007, 72 (19), 7307-7312). The tert-butyl carbamate is removed under the reaction conditions. |
| 62 | 4-{[(3R,4S)-3- hydroxypiperidin-4- yl]oxy}-6-(propan-2- yloxy)quinoline-7- carboxamide | 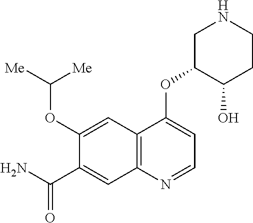 [SMILES: CC(C)Oc1cc2c(O[C@@H]3CNCC[C@@H]3O)ccnc2cc1C(N)=O] | ¹H NMR (400 MHz, DMSO-d₆): δ 8.90- 8.85 (m, 1H), 8.35 (s. 1H), 7.75 (s, 1H), 7.50-7.45 (m, 1H), 5.25-5.10 (m, 1H), 5.05-4.95 (m, 1H), 4.30-4.28 (m, 1H), 3.35-3.10 (m, 4H), 2.30-2.20 (m, 1H), 2.15-2.05 (m, 1H), 1.45-1.35 (m, 6H). MS m/z 346 [M + H]⁺; Step 1 using 4- chloro-6-isopropoxyquinoline-7- carboxamide (Preparation 38) and tert- butyl (3S,4R)-3,4-dihydroxypiperidine-1- carboxylate (J. Org. Chem. 2007, 72 (19), 7307-7312). The tert-butyl carbamate is removed under the reaction conditions. Purified by preparative HPLC. |
| 63 | 4-{[(3S,4S)-3- fluoropiperidin-4- yl]oxy}-6-(propan-2- yloxy)quinoline-7- carboxamide | 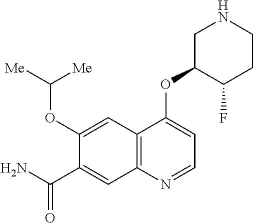 [SMILES: CC(C)Oc1cc2c(O[C@H]3CNCC[C@@H]3F)ccnc2cc1C(N)=O] | Using 4-chloro-6-isopropoxyquinoline-7- carboxamide (Preparation 38) and tert- butyl (3S,4S)-4-fluoro-3- hydroxypiperidine-1-carboxylate. |

### Example 64

### 1-(piperidin-3-yloxy)-7-(propan-2-yloxy)isoquinoline-6-carboxamide hydrochloride

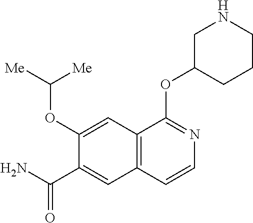 [SMILES: CC(C)Oc1cc2c(OC3CCCNC3)nccc2cc1C(N)=O]

The title compound was prepared as described for Example 53 and Preparations 17 and 3 using 1-chloro-7-isopropoxyisoquinoline-6-carbonitrile (Preparation 35) and tert-butyl-3-hydroxypiperidine-1-carboxylate. The title compound was purified by preparative HPLC. LCMS Method 3: Rt=2.56 min; MS m/z 330 [M+H]⁺

### Example 65

### (R)-6-ethoxy-4-(piperidin-3-yloxy)quinoline-7-carboxamide hydrochloride

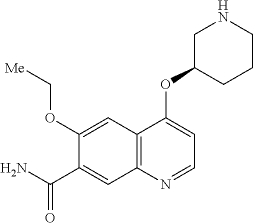 [SMILES: CCOc1cc2c(O[C@@H]3CCCNC3)ccnc2cc1C(N)=O]

Step 1: To a solution of (R)-tert-butyl 3-[(7-carbamoyl-6-fluoroquinolin-4-yl)oxy]piperidine-1-carboxylate (Preparation 15, 150 mg, 0.385 mmol) in THF (3.85 mL) was added EtOH (89 mg, 1.92 mmol) and tBuOK (200 mg, 1.73 mmol) and the reaction mixture was heated to 75° C. for 6 h. The reaction mixture was cooled and extracted into EtOAc. The organic solution was dried and concentrated in vacuo.

Step 2: The residue was dissolved in MeOH and treated with 4M HCl in dioxane. The mixture was stirred at room temperature for 1 h and was then concentrated in vacuo. The residue was triturated with diethyl ether and acetonitrile to afford the title compound as the hydrochloride salt (104 mg, 70%). The material was used without further purification.

### Example 66

### 1-{[(1S,5R,8R)-7-oxo-6-azabicyclo[3.2.1]oct-3-en-8-yl]oxy}-7-(propan-2-yloxy)isoquinoline-6-carboxamide

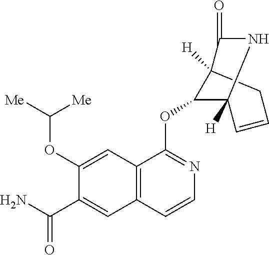 [SMILES: CC(C)Oc1cc2c(O[C@@H]3[C@@H]4CC=C[C@H]3NC4=O)nccc2cc1C(N)=O]

To a solution of (1S,5R,8R)-8-hydroxy-6-azabicyclo[3.2.1]oct-3-en-7-one (ASDI Inc., CAS#1276018-16-6, 400 mg, 2.88 mmol) in DMSO (10.6 mL) was added tBuOK (320 mg, 2.77 mmol) and the reaction mixture was stirred at room temperature for 10 min. 1-Chloro-7-isopropoxyisoquinoline-6-carbonitrile (Preparation 37, 526 mg, 2.13 mmol) was added and the reaction mixture was heated to 75° C. for 35 min. The reaction mixture was cooled, diluted with EtOAc (40 mL) and washed with water (40 mL). The aq layer was further extracted with EtOAc (40 mL), and the organic layers were combined, washed with water (3×40 mL) and brine (40 mL), dried over Na₂SO₄ and concentrated in vacuo. The residue was purified using silica gel column chromatography eluting with 50-100% EtOAc in heptanes. A portion of the residue (75 mg) was dissolved in DMSO (1.43 mL) and treated with K₂CO₃ (119 mg, 0.86 mmol). The reaction mixture was heated to 70° C. and then H₂O₂(30% aq solution, 0.2 mL) was slowly added. The reaction mixture was stirred at 70° C. for 25 min and was then cooled and partitioned between EtOAc (10 mL) and saturated aq NaS₂O₃ solution (10 mL). The aq layer was extracted with EtOAc (2×10 mL) and the combined organic layers were washed with water (2×15 mL) and brine (15 mL), dried over Na₂SO₄ and concentrated in vacuo. The residue was purified using preparative HPLC. LCMS Method 3: Rt=3.51 min; MS m/z 368 [M+H]⁺

### Example 67

### (R)-6-(difluoromethoxy)-4-(piperidin-3-yloxy)quinoline-7-carboxamide

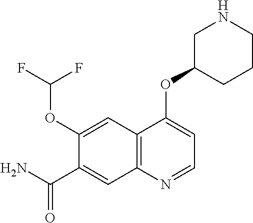 [SMILES: NC(=O)c1cc2nccc(O[C@@H]3CCCNC3)c2cc1OC(F)F]

Step 1: A mixture of (R)-tert-butyl 3-[(7-carbamoyl-6-hydroxyquinolin-4-yl)oxy]piperidine-1-carboxylate (Preparation 7, 209 mg, 0.539 mmol), sodium 2-chloro-2,2-difluoroacetate (161 mg, 1.01 mmol), and cesium carbonate (330 mg, 1.01 mmol) in DMF (5.4 mL) and water (0.5 mL) was stirred at room temperature overnight and then heated to 100° C. for 6 h. The reaction mixture was cooled and partitioned between water and EtOAc. The organic layer was separated, dried over Na₂SO₄ and concentrated in vacuo. The residue was triturated with diethyl ether to afford a white solid.

Step 2: The solid from Step 1 was treated with 4M HCl in dioxane and allowed to stand at room temperature for 2 h before concentrating in vacuo. The residue was purified using reverse phase column chromatography to afford the title compound. ¹H NMR (400 MHz, DMSO-d₆): δ 8.65 (d, 1H), 8.05 (s, 1H), 7.95 (br s, 1H), 7.85 (s, 1H), 7.70 (br s, 1H), 7.50, 7.30 and 7.15 (t, 1H), 7.18 (d, 1H), 4.70-4.60 (m, 1H), 3.20-3.15 (m, 1H), 2.80-2.55 (m, 3H), 2.15-2.05 (m, 1H), 1.80-1.60 (m, 2H), 1.55-1.48 (m, 1H). MS m/z 338 [M+H]⁺

### Example 68

### (R)-6-isopropoxy-2-methyl-4-(piperidin-3-yloxy)quinoline-7-carboxamide trifluoroacetate

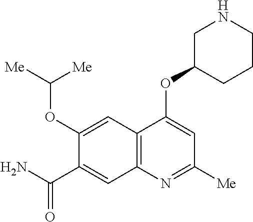 [SMILES: Cc1cc(O[C@@H]2CCCNC2)c2cc(OC(C)C)c(C(N)=O)cc2n1]

The title compound was prepared according to the method described by Preparation 30 using cesium carbonate as base at 70° C. followed by treatment with TFA according to the method described for Example 46 Step 2 using tert-butyl (R)-3-((7-carbamoyl-6-hydroxy-2-methylquinolin-4-yl)oxy)piperidine-1-carboxylate (Preparation 8). The material was used without further purification.

### Example 69

### (R)-3-isopropoxy-5-(piperidin-3-yloxy)-2-naphthamide trifluoroacetate

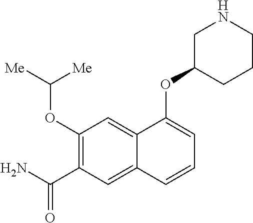 [SMILES: CC(C)Oc1cc2c(O[C@@H]3CCCNC3)cccc2cc1C(N)=O]

Tert-butyl (R)-3-((7-isopropoxy-6-(methoxycarbonyl) naphthalen-1-yl)oxy)piperidine-1-carboxylate (Preparation 25) was treated with ammonium hydroxide as in Preparation 22 Step 2, and the resulting material was then treated with TFA in DCM according to Example 46 Step 2. The residue was triturated with EtOAc to afford the title compound (39% over two steps).

### Example 70

### 1-{[(3R,4S)-4-methylpiperidin-3-yl]oxy}-7-(propan-2-yloxy)isoquinoline-6-carboxamide

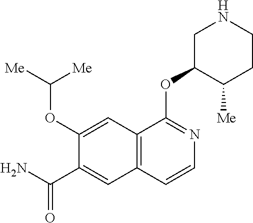 [SMILES: CC(C)Oc1cc2c(O[C@H]3CNCC[C@@H]3C)nccc2cc1C(N)=O]

The title compound was prepared according to Steps 1-3 as described for Example 49. ¹H NMR (400 MHz, DMSO-d₆): δ 8.30 (s, 1H), 8.20 (s, 1H), 7.90-7.88 (m, 1H), 7.75-7.60 (br s, 2H), 7.60 (s, 1H), 7.42-7.38 (m, 1H), 5.00-4.80 (m, 2H), 3.50-3.40 (m, 1H), 3.05-2.95 (m, 1H), 2.70-2.60 (m, 2H), 2.50 (s, 3H), 2.00-1.80 (m, 2H), 1.40-1.30 (m, 1H), 1.00 (d, 6H).

### Example 71

### 4-[(1-methylpiperidin-3-yl)oxyl]-6-(propan-2-yloxy)quinoline-7-carboxamide

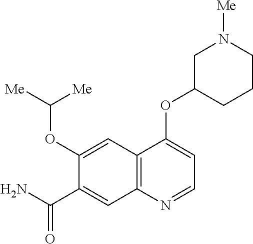 [SMILES: CC(C)Oc1cc2c(OC3CCCN(C)C3)ccnc2cc1C(N)=O]

The title compound was prepared according to the method described for Preparation 9 using 4-chloro-6-isopropoxyquinoline-7-carboxamide (Preparation 38) and 1-methylpiperidin-3-ol. ¹H NMR (400 MHz, DMSO-d₆): δ 8.60 (d, 1H), 8.20 (s, 1H), 7.72 (br s, 1H), 7.68 (br s, 1H), 7.55 (s, 1H), 7.12 (d, 1H), 4.85-4.70 (m, 2H), 3.00-2.90 (m, 1H), 2.60-2.55 (m, 1H), 2.50 (s, 3H), 2.40-2.00 (m, 3H), 1.85-1.75 (m, 1H), 1.70-1.50 (m, 2H), 1.40 (d, 6H). MS m/z 344 [M+H]⁺

### Example 72

### 6-[(2S)-butan-2-yloxy]-4-[(3R)-piperidin-3-yloxy]quinoline-7-carboxamide

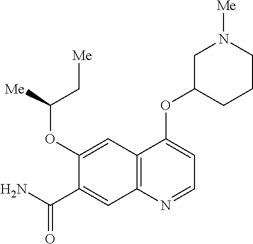 [SMILES: CC[C@H](C)Oc1cc2c(OC3CCCN(C)C3)ccnc2cc1C(N)=O]

The title compound was prepared according to the method described for Library Protocol 5 using (R)-tert-butyl 3-[(7-carbamoyl-6-fluoroquinolin-4-yl)oxy]piperidine-1-carboxylate (Preparation 15) and (S)-butan-2-ol.

### Example 73

### 4-cyano-1-[(3R)-piperidin-3-yloxy]-7-(propan-2-yloxy)isoquinoline-6-carboxamide

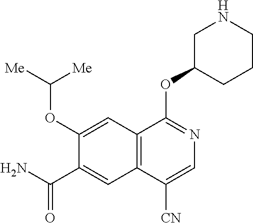 [SMILES: CC(C)Oc1cc2c(O[C@@H]3CCCNC3)ncc(C#N)c2cc1C(N)=O]

The title compound was prepared according to the method described for Example 58 Step 2 using (R)-tert-butyl 3-{[6-carbamoyl-4-cyano-7-(propan-2-yloxy)isoquinolin-1-yl]oxy}piperidine-1-carboxylate (Preparation 2). The residue was purified using preparative HPLC. ¹H NMR (400 MHz, DMSO-d₆): δ 8.57 (s, 1H), 8.20 (s, 1H), 7.90 (br s, 1H), 7.82 (br s, 1H), 7.70 (s, 1H), 5.30-5.20 (m, 1H), 5.00-4.90 (m, 1H), 3.20-3.10 (m, 1H), 2.80-2.70 (m, 2H), 2.65-2.55 (m, 1H), 2.10-2.05 (m, 1H), 1.80-1.70 (m, 2H), 1.55-1.40 (m, 1H), 1.40 (d, 6H).

### Example 74

### 4-{[(3R,4S)-4-methylpiperidin-3-yl]oxy}-6-(propan-2-yloxy)quinoline-7-carboxamide

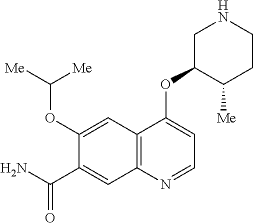 [SMILES: CC(C)Oc1cc2c(O[C@H]3CNCC[C@@H]3C)ccnc2cc1C(N)=O]

The title compound was prepared according to the method described for Example 58 using 4-chloro-6-isopropoxyquinoline-7-carboxamide (Preparation 38) and tert-butyl (3R,4S)-3-hydroxy-4-methylpiperidine-1-carboxylate (US 2011 0092698). The product was purified by preparative HPLC. LCMS Method 3: Rt=2.53 min; MS m/z 344 [M+H]⁺

### Example 75

### 1-[(3S)-piperidin-3-yloxy]-7-(propan-2-yloxy)isoquinoline-6-carboxamide

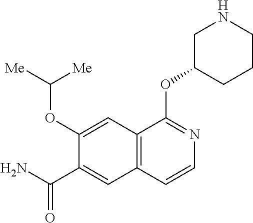 [SMILES: CC(C)Oc1cc2c(O[C@H]3CCCNC3)nccc2cc1C(N)=O]

The title compound may be prepared using the methods described for Preparation 17, Preparation 3, and Example 53 using 1-chloro-7-isopropoxyisoquinoline-6-carbonitrile (Preparation 35) and tert-butyl (S)-3-hydroxypiperidine-1-carboxylate. ¹H NMR (400 MHz, DMSO-d₆): δ 8.20 (s, 1H), 7.90-7.88 (d, 1H), 7.75-7.72 (m, 1H), 7.75 (br s, 2H), 7.45 (d, 1H), 5.60-5.50 (m, 1H), 5.10-5.00 (m, 1H), 3.45-3.40 (m, 2H), 3.30-3.00 (m, 2H), 2.00-1.90 (m, 3H), 1.80-1.70 (m, 1H), 1.40 (d, 6H). MS m/z 330 [M+H]⁺

### Example 76

### 6-(2-methylpropoxy)-4-[(3R)-piperidin-3-yloxy]quinoline-7-carboxamide

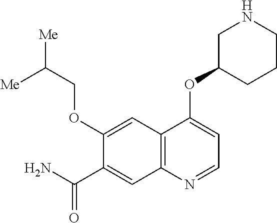 [SMILES: CC(C)COc1cc2c(O[C@@H]3CCCNC3)ccnc2cc1C(N)=O]

The title compound was prepared according to the method described for Example 41 using 1-bromo-2-methylpropane. ¹H NMR (400 MHz, DMSO-d₆): δ 8.65 (d, 1H), 8.15 (s, 1H), 7.75 (br s, 1H), 7.72 (br s, 1H), 7.65 (s, 1H), 7.15 (d, 1H), 5.10-5.00 (m, 1H), 4.08-3.98 (m, 1H), 3.50-3.10 (m, 5H), 2.20-2.10 (m, 1H), 2.05-1.95 (m, 3H), 1.80-1.70 (m, 1H), 1.05 (d, 6H). MS m/z 344 [M+H]⁺

### Example 77

### 6-(propan-2-yloxy)-4-[(3R)-pyrrolidin-3-yloxy]quinoline-7-carboxamide

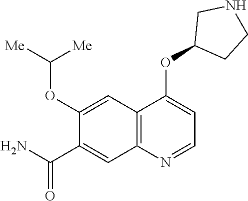 [SMILES: CC(C)Oc1cc2c(O[C@@H]3CCNC3)ccnc2cc1C(N)=O]

The title compound was prepared and purified as described for Library Protocol 6 using N—BOC—(R)-pyrrolidin-3-ol and Purification Method A. LCMS Method 3: LCMS Rt=1.76 min; MS m/z 316 [M+H]⁺

### Example 78

### 4-{[(3R,4S)-4-methylpiperidin-3-yl]oxy}-6-(propan-2-yloxy)quinoline-7-carboxamide

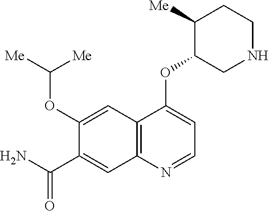 [SMILES: CC(C)Oc1cc2c(O[C@H]3CNCC[C@@H]3C)ccnc2cc1C(N)=O]

The title compound was prepared and purified as described for Library Protocol 6 using tert-butyl (3R,4S)-3-hydroxy-4-methylpiperidine-1-carboxylate and Purification Method A.

LCMS Method 3: LCMS Rt=2.41 min; MS m/z 344 [M+H]⁺

### Example 79

### 7-(propan-2-yloxy)-1-[(3S)-pyrrolidin-3-yloxy]isoquinoline-6-carboxamide

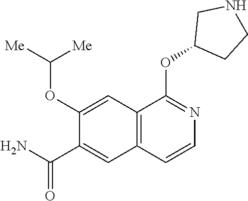 [SMILES: CC(C)Oc1cc2c(O[C@H]3CCNC3)nccc2cc1C(N)=O]

The title compound was prepared and purified as described for Library Protocol 4 using N—BOC—(S)-pyrrolidin-3-ol and using TFA in the final deprotection step. LCMS Method 3: LCMS Rt=2.14 min; MS m/z 316 [M+H]⁺

### Example 80

### 1-[(3-endo)-8-azabicyclo[3.2.1]oct-3-yloxy]-7-(propan-2-yloxy)isoquinoline-6-carboxamide

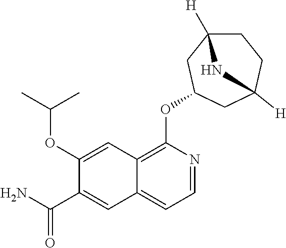 [SMILES: CC(C)Oc1cc2c(O[C@H]3C[C@H]4CC[C@@H](C3)N4)nccc2cc1C(N)=O]

The title compound was prepared and purified as described for Library Protocol 4 using tert-butyl (1R,3R,5S)-3-hydroxy-8-azabicyclo[3.2.1]octane-8-carboxylate and using TFA in the final deprotection step. LCMS Method 3: LCMS Rt=2.21 min; MS m/z 356 [M+H]⁺

### Preparation 1

### Methyl 2-hydroxy-5-nitrobenzoate

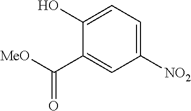 [SMILES: COC(=O)c1cc([N+](=O)[O-])ccc1O]

Concentrated H₂SO₄ (150 mL) was added slowly to a solution of 2-hydroxy-5-nitrobenzoic acid (1000 g, 5.4 mol) in dry MeOH (5 L). After the addition was complete, the mixture was heated to reflux for 24 h. The mixture was filtered. The filtrate was concentrated to afford a solid residue. This residue and the previous precipitate were dissolved in EtOAc and washed with water twice. The EtOAc was then washed with brine, dried over Na₂SO₄, filtered, and concentrated to provide the title compound (900 g, 84%). ¹H NMR (300 MHz, CDCl₃): δ 11.42 (s, 1H), 8.80-8.79 (d, 1H), 8.35-8.31 (dd, 1H), 7.10-7.07 (d, 1H), 4.04 (s, 3H).

### Preparation 2

### (R)-tert-butyl 3-{[6-carbamoyl-4-cyano-7-(propan-2-yloxy)isoquinolin-1-yl]oxy}piperidine-1-carboxylate

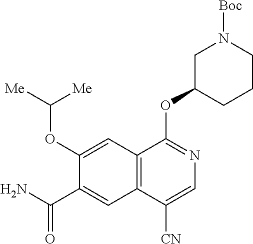 [SMILES: CC(C)Oc1cc2c(O[C@@H]3CCCN(C(=O)OC(C)(C)C)C3)ncc(C#N)c2cc1C(N)=O]

To a solution of (R)-tert-butyl 3-{[4-bromo-6-carbamoyl-7-(propan-2-yloxy)isoquinolin-1-yl]oxy}piperidine-1-carboxylate (Preparation 5, 114 mg, 0.224 mmol) in NMP (2.24 mL) was added zinc cyanide (40 mg, 0.335 mmol) and the mixture was degassed with nitrogen for 15 min. Pd₂(dba)₃ (38 mg, 0.046 mmol) and X-Phos (88 mg, 0.18 mmol) were added and the reaction was heated to 150° C. for 45 min under microwave irradiation. The reaction was diluted with EtOAc and filtered through celite. The filtrate was further diluted with EtOAc (15 mL), washed with saturated aq sodium bicarbonate solution (15 mL), brine (15 mL), dried over Na₂SO₄ and concentrated in vacuo. The residue was purified using silica gel column chromatography eluting with 15-100% EtOAc in heptanes to afford the title compound as a white solid (86 mg, 84%). MS m/z 455 [M+H]⁺

### Preparation 3

### (R)-tert-butyl 3-{[6-carbamoyl-7-(propan-2-yloxy)isoquinolin-1-yl]oxy}piperidine-1-carboxylate

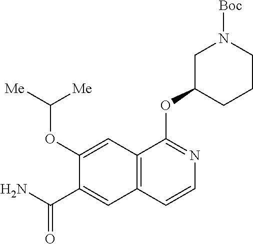 [SMILES: CC(C)Oc1cc2c(O[C@@H]3CCCN(C(=O)OC(C)(C)C)C3)nccc2cc1C(N)=O]

To a solution of (R)-tert-butyl 3-{[6-cyano-7-(propan-2-yloxy)isoquinolin-1-yl]oxy}piperidine-1-carboxylate (Preparation 17, 163 mg, 0.396 mmol) in DMSO (5 mL) was added K₂CO₃ (220 mg, 1.58 mmol) followed by a 30% solution of H₂O₂ in water (1.1 mL). The reaction was heated to 60° C. for 25 min. The reaction was cooled and diluted with water and saturated aq sodium thiosulfate solution (50 mL). The solution was extracted into EtOAc (2×30 mL), the organic layers were collected, washed with water (2×30 mL), brine (30 mL), dried over Na₂SO₄, filtered and concentrated in vacuo to afford the title compound as a white solid. ¹H NMR (400 MHz, DMSO-d₆): δ 8.30-8.10 (m, 1H), 7.90-7.85 (m, 1H), 7.80-7.60 (m, 2H), 7.55-7.45 (s, 1H), 7.40 (d, 1H), 5.30-5.20 (br m, 1H), 4.80-4.70 (m, 1H), 4.30-4.20 (m, 1H), 4.00-3.90 (m, 1H), 3.30-3.20 (m, 1H), 3.0-2.90 (m, 1H), 2.10-1.90 (m, 3H), 1.60-1.50 (m, 1H), 1.40-1.30 (m, 15H).

Preparations 4-6 were prepared according to the method described for Preparation 3 using the appropriate heteroaryl nitrile as described below.

| Prep. | | | |
|---|---|---|---|
| No. | Name | Structure | Data/Starting Material |
| 4 | (R)-tert-butyl 3-{[6- carbamoyl-4-methyl- 7-(propan-2- yloxy)isoquinolin-1- yl]oxy}piperidine-1- carboxylate | 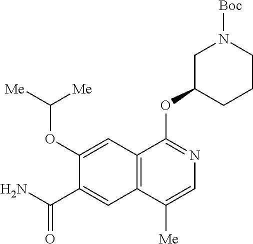 [SMILES: Cc1cnc(O[C@@H]2CCCN(C(=O)OC(C)(C)C)C2)c2cc(OC(C)C)c(C(N)=O)cc12] | MS m/z 445 [M + H]⁺ Using (R)-tert-butyl 3-{[6-cyano-4- methyl-7-(propan-2-yloxy)isoquinolin- 1-yl]oxy}piperidine-1-carboxylate (Preparation 18). |
| 5 | (R)-tert-butyl 3-{[4- bromo-6-carbamoyl- 7-(propan-2- yloxy)isoquinolin-1- yl]oxy}piperidine-1- carboxylate | 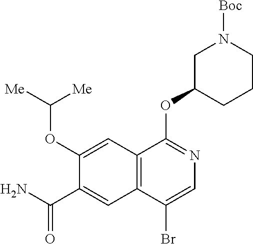 [SMILES: CC(C)Oc1cc2c(O[C@@H]3CCCN(C(=O)OC(C)(C)C)C3)ncc(Br)c2cc1C(N)=O] | ¹H NMR (400 MHz, DMSO-d₆): δ 8.30 (br s, 1H), 8.15 (s, 1H), 7.90- 7.85 (br s, 1H), 7.80-7.75 (br s, 1H), 7.60 (s, 1H), 5.75-5.60 (m, 1H), 4.85- 4.75 (m, 1H), 4.30-4.20 (m, 1H), 4.00-3.90 (m, 1H), 3.25-3.20 (m, 1H), 3.95-3.90 (br m, 1H), 2.20-1.90 (m, 3H), 1.60-1.30 (m. 7H), 0.75 (br s, 9H). Using (R)-tert-butyl 3-{[4-bromo-6- cyano-7-(propan-2-yloxy)isoquinolin- 1-yl]oxy}piperidine-1-carboxylate (Preparation 20). |
| 6 | (R)-tert-butyl 3-{[6- carbamoyl-4-chloro-7- (propan-2- yloxy)isoquinolin-1- yl]oxy}piperidine-1- carboxylate | 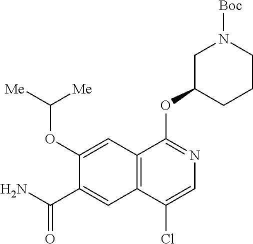 [SMILES: CC(C)Oc1cc2c(O[C@@H]3CCCN(C(=O)OC(C)(C)C)C3)ncc(Cl)c2cc1C(N)=O] | MS m/z 464 [M + H]⁺ Using (R)-tert-butyl 3-{[4-chloro-6- cyano-7-(propan-2-yloxy)isoquinolin- 1-yl]oxy}piperidine-1-carboxylate (Preparation 19). |

### Preparation 7

### (R)-tert-butyl 3-[(7-carbamoyl-6-hydroxyquinolin-4-yl)oxy]piperidine-1-carboxylate

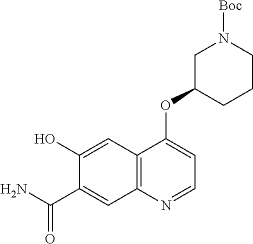 [SMILES: CC(C)(C)OC(=O)N1CCC[C@@H](Oc2ccnc3cc(C(N)=O)c(O)cc23)C1]

To a solution of (R)-tert-butyl 3-{[6-(benzyloxy)-7-carbamoylquinolin-4-yl]oxy}piperidine-1-carboxylate (Preparation 12, 6.14 g, 12.8 mmol) in MeOH (100 mL) was added palladium hydroxide on carbon (400 mg, 2.8 mmol). Hydrogen was bubbled into the solution from a balloon for three min. The reaction was heated at 60° C. for 1.75 h. Nitrogen was bubbled through the solution for three min followed by filtering through Celite. The filtrate was concentrated in vacuo and the resulting solid triturated with diethyl ether to afford the title compound that was taken on directly to the next step (4.24 g, 77%).

### Preparation 8

### tert-butyl (R)-3-((7-carbamoyl-6-hydroxy-2-methylquinolin-4-yl)oxy)piperidine-1-carboxylate

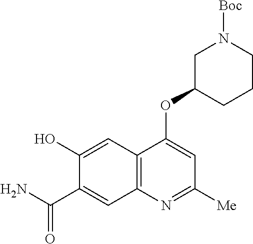 [SMILES: Cc1cc(O[C@@H]2CCCN(C(=O)OC(C)(C)C)C2)c2cc(O)c(C(N)=O)cc2n1]

The title compound was prepared according to the method described by Preparation 7 using tert-butyl (R)-3-((6-(benzyloxy)-7-carbamoyl-2-methylquinolin-4-yl)oxy)piperidine-1-carboxylate (Preparation 13). The reaction was filtered, concentrated in vacuo and taken on directly to the next step.

### Preparation 9

### (R)-tert-butyl 3-{[7-carbamoyl-6-(propan-2-yloxy)quinolin-4-yl]oxy}piperidine-1-carboxylate

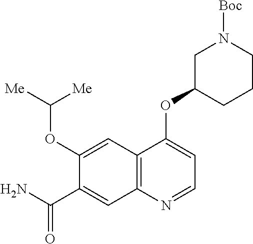 [SMILES: CC(C)Oc1cc2c(O[C@@H]3CCCN(C(=O)OC(C)(C)C)C3)ccnc2cc1C(N)=O]

tBuOK (4.2 g, 39 mmol) was added to a solution of (R)—N—BOC-3-hydroxypiperidine (3.8 g, 19 mmol) in DMSO (10 mL) at room temperature for 10 min. 4-chloro-6-isopropoxyquinoline-7-carboxamide (Preparation 38, 5 g, 19 mmol) in DMSO (5 mL) was added dropwise to the reaction mixture. The reaction mixture was stirred at 60° C. for 4 h. Water was added to reaction mixture which was then extracted with EtOAc. The organic layer was concentrated and purified by column chromatography to obtain the title compound (7.5 g, 92%). ¹H NMR (400 MHz, DMSO-d₆): δ 8.60 (d, 1H), 8.20 (s, 1H), 7.70 (s, 2H), 7.50 (s, 1H), 7.10 (d, 1H), 4.80 (s, 1H), 4.70 (t, 1H), 4.20 (s, 1H), 3.90 (s, 1H), 3.30 (d, 1H), 2.90 (s, 1H), 2.00 (m, 4H), 1.50 (s, 1H), 1.30 (s, 9H), 0.80 (s, 6H). MS m/z 430 [M+H]⁺

Preparations 10-13 were prepared according to the method described by Preparation 9 using the appropriate alcohol and heteroaryl chloride as described below:

| Prep. | | | |
|---|---|---|---|
| No. | Name | Structure | Data/Starting Material |
| 10 | (S)-tert-butyl 3-{[7- carbamoyl-6-(propan- 2-yloxy)quinolin-4- yl]oxy}pyrrolidine-1- carboxylate | 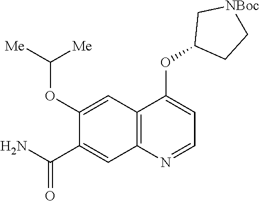 [SMILES: CC(C)Oc1cc2c(O[C@H]3CCN(C(=O)OC(C)(C)C)C3)ccnc2cc1C(N)=O] | MS m/z 416 [M + H]⁺; ¹H NMR (400 MHz, DMSO-d₆): δ ppm 8.60 (s, 1H), 8.10 (s, 1H), 7.80-7.60 (br s, 2H), 7.45 (d, 1H), 7.10-7.00 (m, 1H), 5.30- 5.25 (m, 1H), 4.90-4.70 (m, 1H), 3.80-3.40 (m, 4H), 2.40-2.20 (m, 2H), 1.40-1.35 (m, 15H); 4-chloro-6- isopropoxyquinoline-7-carboxamide (Preparation 38) and tert-butyl (S)-3- hydroxypyrrolidine-1-carboxylate. |
| 11 | (R)-tert-butyl 3-{[7- carbamoyl-6-(propan- 2-yloxy)quinolin-4- yl]oxy}pyrrolidine-1- carboxylate | 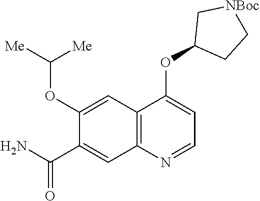 [SMILES: CC(C)Oc1cc2c(O[C@@H]3CCN(C(=O)OC(C)(C)C)C3)ccnc2cc1C(N)=O] | MS m/z 416 [M + H]⁺; ¹H NMR (400 MHz, DMSO-d₆): δ 8.65 (s, 1H), 8.20 (s, 1H), 7.80-7.60 (br s, 2H), 7.45 (d, 1H), 7.10-7.00 (m, 1H), 5.40-5.30 (m, 1H), 4.90-4.70 (m, 1H), 3.80-3.40 (m, 4H), 2.40-2.15 (m, 2H), 1.40-1.35 (m, 15H); 4-chloro-6- isopropoxyquinoline-7-carboxamide (Preparation 38) and tert-butyl (R)- 3-hydroxypyrrolidine-1-carboxylate. |
| 12 | tert-butyl 3-{[6- (benzyloxy)-7- carbamoylquinolin-4- yl]oxy}piperidine-1- carboxylate | 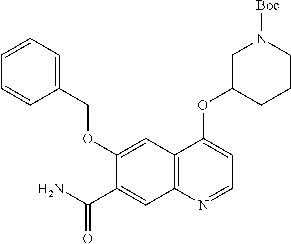 [SMILES: CC(C)(C)OC(=O)N1CCCC(Oc2ccnc3cc(C(N)=O)c(OCc4ccccc4)cc23)C1] | 6-(benzyloxy)-4-chloroquinoline-7- carboxamide (Preparation 22) and N-BOC-3-hydroxypiperidine. Taken on directly to the next step. |
| 13 | tert-butyl (R)-3-((6- (benzyloxy)-7- carbamoyl-2- methylquinolin-4- yl)oxy)piperidine-1- carboxylate | 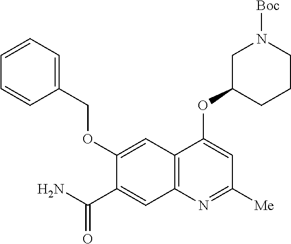 [SMILES: Cc1cc(O[C@@H]2CCCN(C(=O)OC(C)(C)C)C2)c2cc(OCc3ccccc3)c(C(N)=O)cc2n1] | 6-(benzyloxy)-4-chloro-2- methylquinoline-7-carboxamide (Preparation 23) and (R)-N-BOC-3- hydroxypiperidine. |

### Preparation 14

### (R)-tert-butyl 3-{[6-carbamoyl-4-chloro-7-(propan-2-yloxy)isoquinolin-1-yl]oxy}piperidine-1-carboxylate

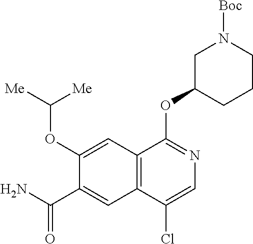 [SMILES: CC(C)Oc1cc2c(O[C@@H]3CCCN(C(=O)OC(C)(C)C)C3)ncc(Cl)c2cc1C(N)=O]

The title compound was prepared according to the method described by Preparation 3 using (R)-tert-butyl 3-{[4-chloro-6-cyano-7-(propan-2-yloxy)isoquinolin-1-yl]oxy}piperidine-1-carboxylate (Preparation 19). ¹H NMR (400 MHz, DMSO-d₆): δ 8.40-8.30 (br m, 1H), 8.10 (s, 1H), 7.85 (br s, 1H), 7.75 (br s, 1H), 7.60 (s, 1H), 5.20 (br m, 1H), 4.90-4.80 (m, 1H), 4.30-4.20 (m, 1H), 3.95-3.80 (m, 1H), 3.30-3.15 (m, 1H), 2.95-2.90 (m, 1H), 2.10-1.80 (m, 3H), 1.60-1.10 (m, 15H). MS m/z 464 [M+H]⁺

### Preparation 15

### (R)-tert-butyl 3-[(7-carbamoyl-6-fluoroquinolin-4-yl)oxy]piperidine-1-carboxylate

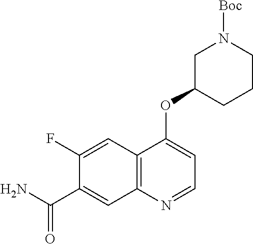 [SMILES: CC(C)(C)OC(=O)N1CCC[C@@H](Oc2ccnc3cc(C(N)=O)c(F)cc23)C1]

To a solution of (R)-4-{[1-(tert-butoxycarbonyl)piperidin-3-yl]oxy}-6-fluoroquinoline-7-carboxylic acid (Preparation 16, 2.6 g, 6.66 mmol) in THF (20 mL) was added TEA (1.41 mL, 9.99 mmol) followed by BOP (3.57 g, 7.99 mmol) and ammonium hydroxide (2 mL). The reaction was stirred at room temperature until reaction completion. The reaction was concentrated in vacuo and partitioned between EtOAc and water. The organic layer was collected, washed with water, brine, dried over Na₂SO₄, filtered and concentrated in vacuo. The resulting solid was triturated with EtOAc to afford the title compound. ¹H NMR (400 MHz, DMSO-d₆): δ 8.75 (d, 1H), 8.25-8.10 (br m, 1H), 8.05-7.95 (m, 1H), 7.80-7.55 (m, 2H), 7.20-7.15 (m, 1H), 4.90-4.85 (m, 1H), 4.20-4.05 (m, 1H), 3.90-3.60 (m, 1H), 3.25-2.90 (m, 2H), 2.05-1.30 (m, 4H), 0.90 (s, 9H).

### Preparation 16

### (R)-4-{[1-(tert-butoxycarbonyl) piperidin-3-yl]oxy}-6-fluoroquinoline-7-carboxylic acid

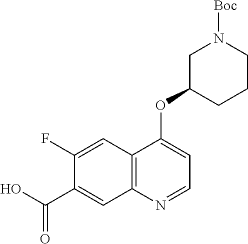 [SMILES: CC(C)(C)OC(=O)N1CCC[C@@H](Oc2ccnc3cc(C(=O)O)c(F)cc23)C1]

To a solution of diisopropylamine (4.33 mL, 30.5 mmol) in THF (50 mL) at 0° C. under nitrogen was added a 2M solution of n-BuLi in THF (13.3 mL, 26.6 mmol). The reaction was stirred at room temperature for 5 min before cooling to −78° C. This LDA solution was added via cannula to a solution of (R)-tert-butyl 3-[(6-fluoroquinolin-4-yl)oxy]piperidine-1-carboxylate (Preparation 21, 8.80 g, 25.40 mmol) in THF (50 mL) also cooled to −78° C. The reaction was stirred at this temperature for 90 min before bubbling CO₂ through the mixture for 5 min at −78° C. and continuing while the reaction was allowed to warm to room temperature. The reaction was concentrated in vacuo and the residue partitioned between diethyl ether (100 mL) and water (100 mL). The aq layer was collected, acidified with 10% (aq) citric acid and extracted into EtOAc. The organic layer was collected, washed with water, brine, dried over Na₂SO₄, filtered and concentrated in vacuo. The resulting solid was triturated with diethyl ether to afford the title compound (2.70 g, 27%) which was taken on directly to the next step.

### Preparation 17

### (R)-tert-butyl 3-{[6-cyano-7-(propan-2-yloxy)isoquinolin-1-yl]oxy}piperidine-1-carboxylate

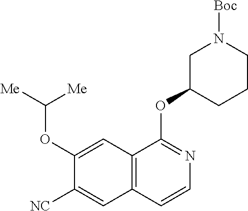 [SMILES: CC(C)Oc1cc2c(O[C@@H]3CCCN(C(=O)OC(C)(C)C)C3)nccc2cc1C#N]

To a solution of 1-chloro-7-isopropoxyisoquinoline-6-carbonitrile (Preparation 35, 123 mg, 0.499 mmol) in DMSO (5 mL) was added tert-butyl (R)-3-hydroxypiperidine-1-carboxylate (132 mg, 0.623 mmol) followed by tBuOK (72 mg, 0.623 mmol). The reaction was heated to 60° C. for 2 h before cooling. The reaction was diluted with water (30 mL) and extracted with EtOAc (2×30 mL). The organic layers were collected, washed with water (2×30 mL), brine (30 mL), dried over Na₂SO₄, filtered and concentrated in vacuo. The residue was purified using silica gel column chromatography eluting with 15-50% EtOAc in heptanes to afford the title compound as a white foam (163 mg, 79%). ¹H NMR (400 MHz, DMSO-d₆): δ 8.55-8.50 (br s, 1H), 8.00-7.95 (m, 1H), 7.60 (s, 1H), 7.40-7.35 (m, 1H), 5.30-5.20 (m, 1H), 4.90-4.80 (m, 1H), 4.30-3.90 (m, 1H), 3.30-3.20 (m, 1H), 3.00-2.90 (m, 1H), 2.10-1.90 (m, 3H), 1.60-1.50 (m, 1H), 1.40-1.30 (m, 16H).

### Preparation 18

### (R)-tert-butyl 3-{[6-cyano-4-methyl-7-(propan-2-yloxy)isoquinolin-1-yl]oxy}piperidine-1-carboxylate

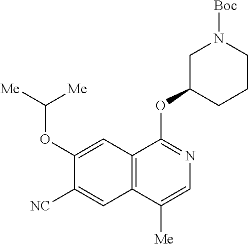 [SMILES: Cc1cnc(O[C@@H]2CCCN(C(=O)OC(C)(C)C)C2)c2cc(OC(C)C)c(C#N)cc12]

A mixture of (R)-tert-butyl 3-{[4-bromo-6-cyano-7-(propan-2-yloxy)isoquinolin-1-yl]oxy}piperidine-1-carboxylate (Preparation 20, 490 mg, 0.285 mmol), Pd(PPh₃)Cl₂ (40 mg, 0.057 mmol), K₂CO₃ (119 mg, 0.855 mmol) and methyltrifluoroborate potassium salt (53 mg, 0.427 mmol) was suspended in degassed MeCN (1.42 mL) and water (0.8 mL) and the reaction was heated to 125° C. for 30 min under microwave irradiation. The reaction mixture was cooled to room temperature and diluted with EtOAc (10 mL) and filtered. The filtrate was washed with water and brine, dried over Na₂SO₄ and concentrated in vacuo. The residue was purified using silica gel column chromatography eluting with 15-100% EtOAc in heptanes to afford the title compound (67 mg, 55%). MS m/z 445 [M+H]⁺

### Preparation 19

### (R)-tert-butyl 3-{[4-chloro-6-cyano-7-(propan-2-yloxy)isoquinolin-1-yl]oxy}piperidine-1-carboxylate

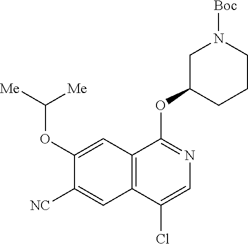 [SMILES: CC(C)Oc1cc2c(O[C@@H]3CCCN(C(=O)OC(C)(C)C)C3)ncc(Cl)c2cc1C#N]

To a solution of (R)-tert-butyl 3-{[6-cyano-7-(propan-2-yloxy)isoquinolin-1-yl]oxy}piperidine-1-carboxylate (Preparation 17, 150 mg, 0.365 mmol) in acetonitrile (7.3 mL) was added NCS (59 mg, 0.438 mmol) and the reaction was heated to 60° C. for 2 h. The reaction was cooled, concentrated in vacuo and purified using silica gel column chromatography eluting with 10-50% EtOAc in heptanes to afford the title compound as a white foam (135 mg, 83%).

¹H NMR (400 MHz, DMSO-d₆): δ 8.55 (brs, 1H), 8.15 (s, 1H), 7.70 (s, 1H), 5.20 (m, 1H), 4.95-4.90 (m, 1H), 4.30-4.19 (m, 1H), 4.00-3.90 (m, 1H), 3.30-3.20 (m, 1H), 3.00-2.90 (m, 1H), 2.10-1.90 (m, 4H), 1.60-1.30 (m, 15H).

### Preparation 20

### (R)-tert-butyl 3-{[4-bromo-6-cyano-7-(propan-2-yloxy)isoquinolin-1-yl]oxy}piperidine-1-carboxylate

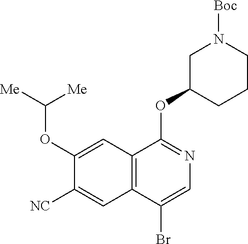 [SMILES: CC(C)Oc1cc2c(O[C@@H]3CCCN(C(=O)OC(C)(C)C)C3)ncc(Br)c2cc1C#N]

The title compound was prepared from (R)-tert-butyl 3-{[6-cyano-7-(propan-2-yloxy)isoquinolin-1-yl]oxy}piperidine-1-carboxylate (Preparation 17) according to the method described for Preparation 19 using NBS at room temperature for 1 h. ¹H NMR (400 MHz, DMSO-d₆): δ 8.50-8.40 (br m, 1H), 8.25 (s, 1H), 7.65 (s, 1H), 5.25-5.20 (m, 1H), 4.95-4.85 (m, 1H), 4.25-4.19 (br m, 0.5H), 3.95-3.85 (br m, 0.5H), 3.30-3.20 (m, 3H), 2.10-1.90 (m, 4H), 1.60-1.25 (m, 7H), 0.75 (br s, 9H).

### Preparation 21

### (R)-tert-butyl 3-[(6-fluoroquinolin-4-yl)oxy]piperidine-1-carboxylate

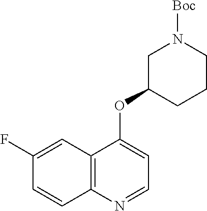 [SMILES: CC(C)(C)OC(=O)N1CCC[C@@H](Oc2ccnc3ccc(F)cc23)C1]

To a solution of tert-butyl (R)-3-hydroxypiperidine-1-carboxylate (6.58 g, 32.7 mmol) in DMSO (40 mL) was added tBuOK (3.75 g, 32.7 mmol) and the solution was stirred at room temperature for 10 min. The solution was then transferred via cannula to a solution of 4-chloro-6-fluoroquinoline (5.4 g, 29.7 mmol) in DMSO (60 mL), and the reaction was heated at 60° C. for 45 min. The reaction was quenched by the addition of ice-water and extracted into EtOAc. The organic layer was collected, washed with water, brine, dried over Na₂SO₄, filtered and concentrated in vacuo. The residue was purified using silica gel column chromatography eluting with 20-60% EtOAc in heptanes to afford the title compound as a solid (8.80 g, 85%) and used directly in Preparation 16.

### Preparation 22

### 6-(benzyloxy)-4-chloroquinoline-7-carboxamide

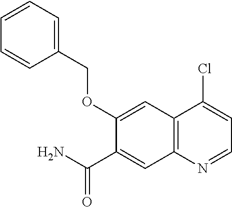 [SMILES: NC(=O)c1cc2nccc(Cl)c2cc1OCc1ccccc1]

Step 1: A mixture of methyl 6-(benzyloxy)-4-hydroxyquinoline-7-carboxylate (Preparation 24, 3.39 g, 10.96 mmol) in phosphorus oxychloride (30 mL) was heated to 100° C. for 2 h. The reaction was cooled and poured into ice-water (200 g-100 mL). The mixture was extracted into EtOAc (250 mL). The organic layer was collected, washed with saturated aq sodium bicarbonate solution (150 mL), water (150 mL), brine (150 mL), dried over Na₂SO₄ and concentrated in vacuo. The residue was purified using silica gel column chromatography, eluting with 8-33% EtOAc in hexanes.

Step 2: The resulting yellow solid (2.34 g) was suspended in ethanol (60 mL) and treated with 28-30% ammonium hydroxide solution. The reaction was heated in a sealed vessel at 70° C. for 16 h. The reaction was cooled, concentrated in vacuo and the resulting solid triturated with diethyl ether to afford the title compound (1.90 g, 58% over two steps), which was used without further purification in Preparation 12.

### Preparation 23

### 6-(benzyloxy)-4-chloro-2-methylquinoline-7-carboxamide

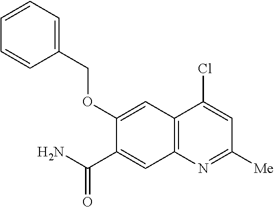 [SMILES: Cc1cc(Cl)c2cc(OCc3ccccc3)c(C(N)=O)cc2n1]

The title compound was prepared according to the method described by Preparation 22 using methyl 6-(benzyloxy)-4-chloro-2-methylquinoline-7-carboxylate (Preparation 36). The material was used without further purification in Preparation 13.

### Preparation 24

### Methyl 6-(benzyloxy)-4-hydroxyquinoline-7-carboxylate

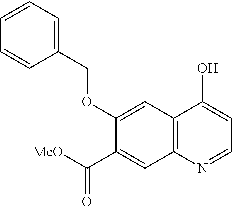 [SMILES: COC(=O)c1cc2nccc(O)c2cc1OCc1ccccc1]

To a solution of methyl 5-amino-2-(benzyloxy)benzoate (12 g, 46.64 mmol) in ethanol (80 mL) was added Meldrum's acid (7.78 g, 51.3 mmol) followed by triethylorthoformate (7.68 g, 51.3 mmol) and the reaction was heated to reflux for 1 h. The reaction was cooled in an ice-water bath and the resulting precipitate was filtered and washed with cold EtOH. The solid was added to hot (250-260° C.) Dowtherm (400 mL) in portions with vigorous gas evolution. After complete addition, the reaction was stirred for 5 min and then allowed to cool to room temperature. Hexanes (250 mL) was added to the mixture and the solvent was decanted. The residue was washed with diethyl ether and decanted. The remaining solid was dissolved in 3% MeOH in DCM and purified using silica gel column chromatography eluting with 5-10% MeOH in DCM to afford the title compound (3.44 g, 35%). The material was used without further purification in Preparation 22.

### Preparation 25

### tert-butyl (R)-3-((7-isopropoxy-6-(methoxycarbonyl)naphthalen-1-yl)oxy)piperidine-1-carboxylate

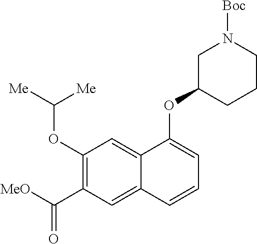 [SMILES: COC(=O)c1cc2cccc(O[C@@H]3CCCN(C(=O)OC(C)(C)C)C3)c2cc1OC(C)C]

The title compound was prepared according to the method described for Preparation 30 using tert-butyl (R)-3-((7-hydroxy-6-(methoxycarbonyl)naphthalen-1-yl)oxy)piperidine-1-carboxylate (Preparation 26) and cesium carbonate. The residue was triturated with 25% EtOAc in heptanes. The material was used without further purification in the next step.

### Preparation 26

### tert-butyl (R)-3-((7-hydroxy-6-(methoxycarbonyl)naphthalen-1-yl)oxy)piperidine-1-carboxylate

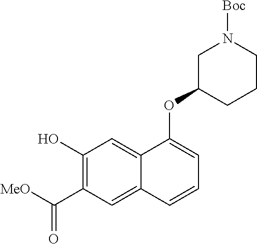 [SMILES: COC(=O)c1cc2cccc(O[C@@H]3CCCN(C(=O)OC(C)(C)C)C3)c2cc1O]

A mixture of methyl 3,5-dihydroxy-2-naphthoate (JACS 2013, 135 (17), 6450-6455, 218 mg, 0.99 mmol), tert-butyl(S)-3-hydroxypiperidine-1-carboxylate (241 mg, 1.20 mmol) DIAD (248 mg, 1.2 mmol) and triphenylphosphine (318 mg, 1.2 mmol) in THF was heated to 60° C. for 2 h. The reaction was cooled, concentrated in vacuo and the residue triturated with cold MeOH to afford the title compound that was taken on directly in the next step.

### Preparation 27

### tert-butyl 3-((6-carbamoyl-7-isopropoxynaphthalen-1-yl)oxy)piperidine-1-carboxylate

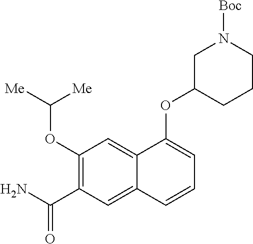 [SMILES: CC(C)Oc1cc2c(OC3CCCN(C(=O)OC(C)(C)C)C3)cccc2cc1C(N)=O]

The title compound was prepared according to the method described for Preparation 26 using tert-butyl-3-hydroxypiperidine-1-carboxylate and 5-hydroxy-3-isopropoxy-2-naphthamide (Preparation 28). The material was used without further purification in the next step.

### Preparation 28

### 5-hydroxy-3-isopropoxy-2-naphthamide

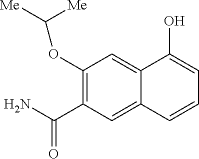 [SMILES: CC(C)Oc1cc2c(O)cccc2cc1C(N)=O]

3-isopropoxy-5-(methoxymethoxy)-2-naphthamide (Preparation 29, 2290 mg, 7.91 mmol) was treated with 6N HCl in THF. Once complete the reaction was concentrated in vacuo to afford the title compound (870 mg, 45%). The material was used without further purification in Preparation 27.

### Preparation 29

### 3-isopropoxy-5-(methoxymethoxy)-2-naphthamide

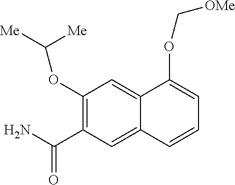 [SMILES: COCOc1cccc2cc(C(N)=O)c(OC(C)C)cc12]

The title compound was prepared according to the method described for Preparation 22 Step 2 using methyl 3-isopropoxy-5-(methoxymethoxy)-2-naphthoate. Methyl 3-isopropoxy-5-(methoxymethoxy)-2-naphthoate was prepared from methyl 3-hydroxy-5-(methoxymethoxy)-2-naphthoate, which was prepared by the method described in Organic Letters, 2012, 14, 1408-1411 using methyl 3,5-dihydroxy-2-naphthoate and chloromethyl methyl ether. The material was used without further purification.

### Preparation 30

### 3-bromo-4-isopropoxybenzaldehyde

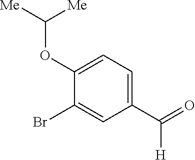 [SMILES: CC(C)Oc1ccc(C=O)cc1Br]

A mixture of 3-bromo-4-hydroxybenzaldehyde (1500 g, 7.5 mol) and K₂CO₃ (1290 g, 9.3 mol) in anhydrous DMSO (15 L) was treated with 2-bromopropane (1010 g, 8.2 mol) and stirred at 55° C. overnight. An additional 200 g (1.6 mol) of 2-bromopropane was added and the reaction mixtures was stirred for approximately an additional 4 h. The reaction mixture was cooled to 30° C., and EtOAc (22.5 L) and water (22.5 L) were added. The EtOAc phase was separated and the aq phase was extracted with EtOAc (2×7.5 L). The combined EtOAc phases were washed with water (2×15 L) followed by brine (15 L), dried over Na₂SO₄, filtered, and concentrated to provide the title compound (1800 g, 99%). ¹H NMR (400 MHz, CDCl₃): δ 9.82 (s, 1H), 8.07-8.08 (d, 1H), 7.76-7.79 (d, 1H), 6.98 (d, 1H), 4.67-4.74 (m, 1H), 1.42-1.44 (d, 6H).

### Preparation 31

### (E)-3-(3-bromo-4-isopropoxyphenyl)acrylic acid

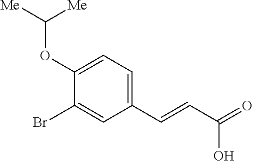 [SMILES: CC(C)Oc1ccc(/C=C/C(=O)O)cc1Br]

3-Bromo-4-isopropoxybenzaldehyde (Preparation 30, 1800 g, 7.4 mol) in anhydrous pyridine (7.56 L) was treated with malonic acid (1002 g, 9.6 mol) and piperidine (316 g, 3.7 mol) and heated to reflux for 2 h. The solvent was removed by distillation under reduced pressure. This was treated with cold water (37.8 L) and stirred for 30 min, then acidified to pH 4 with AcOH (300 mL). The suspension was vigorously stirred for 1 h and then the product was collected by filtration, washed with water (3.6 L) and dried under vacuum to provide the title compound (2014 g, 95%). ¹H NMR (400 MHz, DMSO-d₆): δ 7.94-7.95 (d, 1H), 7.64-7.67 (dd, 1H), 7.48-7.52 (d, 1H), 7.14-7.16 (d, 1H), 6.43-6.47 (d, 1H), 4.71-4.77 (m, 1H), 1.29-1.31 (d, 6H).

### Preparation 32

### (E-3-(3-bromo-4-isopropoxyphenyl)acryloyl azide

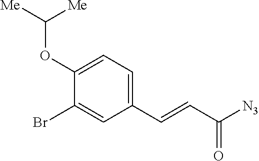 [SMILES: CC(C)Oc1ccc(/C=C/C(=O)N=[N+]=[N-])cc1Br]

To a stirred solution of (E-3-(3-bromo-4-isopropoxyphenyl)acrylic acid (Preparation 31, 1000 g, 3.5 mol) in acetone (17.5 L), Et₃N (355 g, 3.5 mol) was added and the mixture was cooled to −5° C. Ethyl chloroformate (495 g, 4.56 mol) was added dropwise, maintaining the temperature at −5° C. After completion of the addition, the mixture was stirred for 1 h at −5° C.

A solution of sodium azide (342 g, 5.3 mol) in water (1264 mL) was added slowly at −5° C. After the addition of sodium azide solution was complete, the reaction mixture was slowly warmed to 25° C. and stirred for 30 min. The reaction was quenched by the addition of water (50 L) and stirred for 30 min at 25° C. The precipitate was filtered, washed with water (2 L) and dried to provide the title compound (978 g, 90%). ¹H NMR (300 MHz, DMSO-d₆): δ 8.07-8.08 (d, 1H), 7.74-7.77 (dd, 1H), 7.60 (s, 1H), 7.16-7.20 (d, 1H), 6.60-6.66 (d, 1H), 4.74-4.82 (m, 1H), 1.29-1.32 (d, 6H).

### Preparation 33

### 6-bromo-7-isopropoxyisoquinolin-1(2H)-one

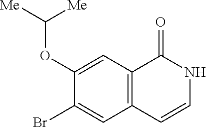 [SMILES: CC(C)Oc1cc2c(=O)[nH]ccc2cc1Br]

To a mixture of diphenyl ether (8 L) and tri n-butyl amine (328 g, 1.77 mol) pre-heated to 230° C., (E-3-(3-bromo-4-isopropoxyphenyl)acryloyl azide (Preparation 32, 550 g, 1.77 mol) dissolved in diphenyl ether (2.5 L) was added while the temperature was maintained at 230° C. After the addition was completed, stirring and heating were continued for 30 min. The reaction mixture was cooled to 25° C. and added slowly to hexane (27.5 L). The resulting slurry was cooled to 0° C. and stirred for 30 min. The crude precipitate was filtered and the precipitate was washed with cold hexane (5.5 L). The wet cake was dried under vacuum.

This reaction was repeated three more times to afford 1064 g of crude material. This was dissolved in THF (5.3 L) at reflux and cooled to 0° C. The slurry was stirred for 30 min and then filtered and the filter cake was dried under vacuum. The filtrate was concentrated and purified by silica gel column chromatography to afford an additional 181 g to provide the title compound (755 g, 46%). ¹H NMR (300 MHz, CDCl₃): δ 8.26-8.24 (d, 1H), 8.16 (s, 1H), 7.65 (s, 1H), 7.56-7.55 (d, 1H), 4.91-4.85 (m, 1H), 1.52-1.51 (d, 6H).

### Preparation 34

### 7-isopropoxy-1-oxo-1,2-dihydroisoquinoline-6-carbonitrile

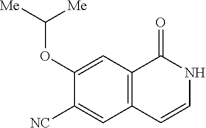 [SMILES: CC(C)Oc1cc2c(=O)[nH]ccc2cc1C#N]

6-Bromo-7-isopropoxyisoquinolin-1(2H)-one (Preparation 33, 490 g, 1.74 mol) and zinc cyanide (265 g, 2.25 mol) were added to dry DMF (9.8 L) and stirred well for 5 min at 25° C. The reaction mixture was degassed with nitrogen for 20 min, after which tetra(kis)triphenylphosphinepalladium (0) (120 g, 0.104 mol) was added and the reaction mixture was stirred for 5 min at 25° C. before being heated to 100° C. The mixture was maintained for 16 h at 100° C. The reaction mixture was cooled to 25° C., diluted with EtOAc (4.9 L), and stirred for 30 min. The mixture was filtered through celite, which was washed with EtOAc (1 L). The combined filtrate was concentrated at a pressure of 10 Torr at 75° C. Water (4.9 L) was added to the residue and the mixture was stirred for 30 min. The precipitate was filtered, washed with water (1 L) and dried under vacuum at 60° C. The precipitate was stirred for 30 min with MTBE (4.9 L) and filtered. This process was repeated twice more, after which the filter cake was washed with MTBE (0.5 L) and dried under vacuum at 60° C. to provide the title compound (390 g, 98%). ¹H NMR (400 MHz, CDCl₃): δ 8.26-8.24 (d, 1H), 8.16 (s, 1H), 7.65 (s, 1H), 7.56-7.54 (d, 1H), 4.99-4.85 (m, 1H), 1.52-1.51 (d, 6H).

### Preparation 35

### 1-chloro-7-isopropoxyisoquinoline-6-carbonitrile

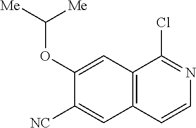 [SMILES: CC(C)Oc1cc2c(Cl)nccc2cc1C#N]

7-Isopropoxy-1-oxo-1,2-dihydroisoquinoline-6-carbonitrile (Preparation 34, 390 g, 1.7 mol) and POCl₃ (10.97 kg, 71.5 mol) were stirred for 5 min at 25° C., then heated to 100° C. and maintained at 100° C. for 30 min. The reaction mixture was cooled to 25° C. and concentrated under reduced pressure at 60° C. The residue was quenched with ice (7.8 kg), then neutralized with 25% K₂CO₃ solution (7.8 L) with stirring to pH=7. The mixture was extracted with DCM (3×5 L), and the combined extracts were washed with 10% NaHCO₃ solution (2×3.9 L). The DCM was separated, dried over Na₂SO₄, filtered, and concentrated. n-Heptane (3.9 L) was added to the residue and the mixture was stirred for 30 min at 25° C. The precipitate was filtered and the filter cake was washed with n-heptane (390 mL) and dried under vacuum at 60° C. to provide the title compound (338 g, 80%). ¹H NMR (300 MHz, DMSO-d₆): δ 8.73 (s, 1H), 8.29-8.31 (d, 1H), 7.89-7.91 (d, 1H), 7.67 (s, 1H), 5.02-5.06 (m, 1H), 1.41-1.43 (d, 6H).

### Preparation 36

### Methyl 6-(benzyloxy)-4-chloro-2-methylquinoline-7-carboxylate

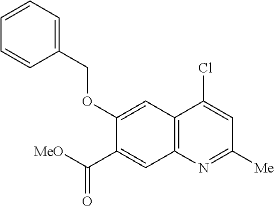 [SMILES: COC(=O)c1cc2nc(C)cc(Cl)c2cc1OCc1ccccc1]

The title compound was prepared according to the method described by Preparations 35 using methyl 6-isopropoxy-2-methyl-4-oxo-1,4-dihydroquinoline-7-carboxylate (Preparation 41). The residue was purified using silica gel column chromatography eluting with 8-33% EtOAc in heptanes. The material was used without further purification in the next step.

### Preparation 37

### 1-chloro-7-isopropoxyisoquinoline-6-carboxamide

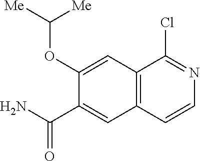 [SMILES: CC(C)Oc1cc2c(Cl)nccc2cc1C(N)=O]

To a solution of 1-chloro-7-isopropoxyisoquinoline-6-carbonitrile (Preparation 35, 485 mg, 1.97 mmol) in DMSO (4 mL) was added K₂CO₃ (1.09 g, 7.86 mmol) followed by a 30% aq solution of H₂O₂(19.7 mL). The reaction was heated to 60° C. for 1 h. The reaction was cooled, diluted with EtOAc and washed with saturated aq Na₂S₂O₃ solution. The aq layer was collected and extracted with EtOAc. The organic layers were combined, washed with saturated aq Na₂S₂O₃ solution, brine, dried over magnesium sulfate and concentrated in vacuo. The resulting off-white solid was dried to afford the title compound (470 mg, 90%). ¹H NMR (400 MHz, DMSO-d₆): δ 8.30 (s, 1H), 8.25 (d, 1H), 7.95 (d, 1H), 7.75 (br s, 2H), 7.58 (s, 1H), 4.98-4.90 (m, 1H), 1.40 (d, 6H).

### Preparation 38

### 4-chloro-6-isopropoxyquinoline-7-carboxamide

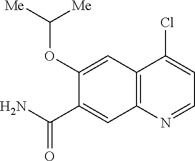 [SMILES: CC(C)Oc1cc2c(Cl)ccnc2cc1C(N)=O]

A mixture of 6-isopropoxy-4-oxo-1,4-dihydroquinoline-7-carboxylic acid (Preparation 39, 129 g, 520 mmol) and POCl₃ (2 L) was heated under reflux for 6 h. The mixture was concentrated under reduced pressure to give a dark oil which was immediately treated with 10 L of a saturated solution of ammonia in dioxane at 0° C. The mixture was then stirred at 25° C. overnight. The reaction mixture was filtered and the filtrate was concentrated. The residue was diluted with DCM (1 L) and washed with water. The DCM extract was concentrated to obtain a solid residue which was purified by trituration with diethyl ether to provide the title compound (55 g, 40%). ¹H NMR (400 MHz, DMSO-d₆): δ 8.90 (s, 1H), 8.72-8.71 (d, 1H), 7.80 (br s, 1H), 7.50 (s, 1H), 4.90 (m, 1H), 1.47 (d, 6H).

### Preparation 39

### 6-isopropoxy-4-oxo-1,4-dihydroquinoline-7-carboxylic acid

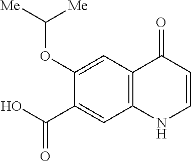 [SMILES: CC(C)Oc1cc2c(=O)cc[nH]c2cc1C(=O)O]

Methyl 6-isopropoxy-4-oxo-1,4-dihydroquinoline-7-carboxylate (Preparation 40, 311 g, 1.19 mol) was added to THF (1.55 L) and water (1.55 L). Lithium hydroxide monohydrate (199 g, 4.76 mol) was added and the mixture was stirred at 25° C. overnight. The reaction mixture was diluted with water and extracted with EtOAc 4 times until the methyl 6-isopropoxy-4-oxo-1,4-dihydroquinoline-5-carboxylate was absent from the aq phase. The aq layer was adjusted to pH 1 by the addition of 1M HCl and the aq layer was extracted with EtOAc. The EtOAc extracts were dried over Na₂SO₄, filtered, and concentrated to provide the title compound (129 g, 44%).

¹H NMR (400 MHz, DMSO-d₆): δ 13.00-11.60 (br s, 1H), 8.06-8.04 (d, 1H), 7.84 (s, 1H), 7.60 (s, 1H), 6.22-6.20 (d, 1H), 4.73-4.69 (m, 1H), 1.32-1.30 (d, 6H).

### Preparation 40

### Methyl 6-isopropoxy-4-oxo-1,4-dihydroquinoline-7-carboxylate

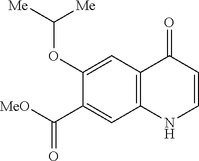 [SMILES: COC(=O)c1cc2[nH]ccc(=O)c2cc1OC(C)C]

Methyl 5-(((2,2-dimethyl-4,6-dioxo-1,3-dioxan-5-ylidene)methyl)amino)-2-isopropoxybenzoate (Preparation 42, 20 g, 55 mmol) was added in portions to pre-heated Dowtherm A (480 mL) at 240° C. Heating and stirring at that temperature were continued for approximately an additional 5 min after completion of the addition. The mixture was cooled to 20° C. and purified by silica gel column chromatography to give 6.2 g of a mixture of the title compound and methyl 6-isopropoxy-4-oxo-1,4-dihydroquinoline-5-carboxylate. This procedure was repeated 29 times more on the same scale to provide a mixture of the title compound and methyl 6-isopropoxy-4-oxo-1,4-dihydroquinoline-5-carboxylate (311 g, 72%).

¹H NMR (400 MHz, DMSO-d₆): δ 11.90 (s, 2H), 7.94-7.92 (d, 1H), 7.87-7.86 (d, 1H), 7.83 (s, 1H), 7.62-7.54 (m, 3H), 6.02-6.01 (d, 1H), 5.93-5.91 (d, 1H), 4.72-4.66 (m, 1H), 4.62-4.56 (m, 1H), 3.85 (s, 3H), 3.76 (s, 3H), 1.31-1.29 (d, 6H), 1.22-1.20 (d, 6H).

### Preparation 41

### Methyl 6-isopropoxy-2-methyl-4-oxo-1,4-dihydroquinoline-7-carboxylate

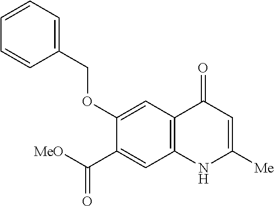 [SMILES: COC(=O)c1cc2[nH]c(C)cc(=O)c2cc1OCc1ccccc1]

The title compound was prepared according to the method described for Preparation 40 using methyl 2-(benzyloxy)-5-((1-(2,2-dimethyl-4,6-dioxo-1,3-dioxan-5-ylidene)ethyl)amino)benzoate (Preparation 43). The residue was purified using silica gel column chromatography eluting with 1-5% MeOH in DCM. The material was used without further purification in the next step.

### Preparation 42

### Methyl 5-(((2,2-dimethyl-4,6-dioxo-1,3-dioxan-5-ylidene)methyl)amino)-2-isopropoxybenzoate

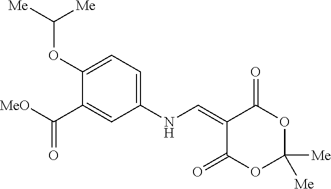 [SMILES: COC(=O)c1cc(NC=C2C(=O)OC(C)(C)OC2=O)ccc1OC(C)C]

A mixture of methyl 5-amino-2-isopropoxybenzoate (Preparation 44, 600 g, 2.88 mol), Meldrum's acid (640 g, 3.44 mol), and triethyl orthoformate (560 g, 3.44 mol) in EtOH (7 L) was heated at reflux overnight. The mixture was cooled to 20° C. and the resulting precipitate was collected by filtration to provide the title compound (620 g, 60%). ¹H NMR (300 MHz, CDCl₃): δ 11.17-11.07 (br d, 1H), 8.58-8.53 (d, 1H), 7.70-7.69 (d, 1H), 7.33-7.29 (dd, 1H), 7.05-7.02 (d, 1H), 4.62-4.55 (m, 1H), 2.91 (s, 3H), 1.75 (d, 6H), 1.40-1.38 (d, 6H).

### Preparation 43

### Methyl 2-(benzyloxy)-5-((1-(2,2-dimethyl-4,6-dioxo-1,3-dioxan-5-ylidene)ethyl)amino)benzoate

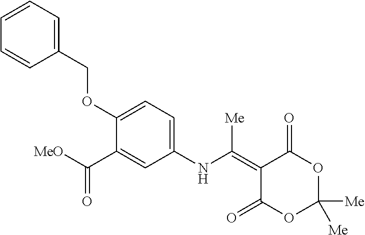 [SMILES: COC(=O)c1cc(NC(C)=C2C(=O)OC(C)(C)OC2=O)ccc1OCc1ccccc1]

The title compound was prepared according to the method described for Preparation 42 using methyl 5-amino-2-(benzyloxy)benzoate (PCT Intl. Appl. 2011 037610) and trimethylorthoacetate. The material was used without further purification in the next step.

### Preparation 44

### Methyl 5-amino-2-isopropoxybenzoate

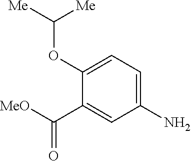 [SMILES: COC(=O)c1cc(N)ccc1OC(C)C]

A solution of methyl 2-isopropoxy-5-nitrobenzoate (Preparation 45, 760 g, 3.17 mol) in EtOH (12 L) was treated with palladium on carbon (76 g) and this mixture was stirred for 1 h at 25° C. under an atmosphere of hydrogen at 50 psi. The mixture was filtered through celite and filter cake was washed with additional EtOH. The filtrate was evaporated to provide the title compound (600 g, 60%). ¹H NMR (400 MHz, DMSO-d₆): δ 6.85-6.83 (d, 1H), 6.79-6.76 (dd, 1H), 4.37-4.31 (m, 1H), 3.86 (s, 3H), 3.60-2.80 (br s, 2H), 1.31-1.29 (d, 6H).

### Preparation 45

### Methyl 2-isopropoxy-5-nitrobenzoate

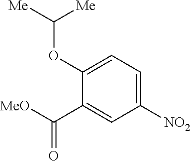 [SMILES: COC(=O)c1cc([N+](=O)[O-])ccc1OC(C)C]

Methyl 2-hydroxy-5-nitrobenzoate (Preparation 1, 900 g, 4.56 mol), triphenylphosphine (1.33 kg, 5.02 mol) and 2-propanol (3.8 kg, 5.02 mol) were dissolved in dry THF (18 L) and cooled to 5° C. DIAD (1.01 kg, 5.02 mol) was added, and the reaction was allowed to warm to 25° C. and was stirred overnight. The mixture was concentrated under reduced pressure and the residue treated with EtOAc (5 L). The undissolved solids that remained were removed by filtration, and the filtrate was evaporated. The residue was purified by column chromatography to provide the title compound (760 g, 70%). ¹H NMR (300 MHz, CDCl₃): δ 8.60 (d, 1H), 8.34-8.29 (dd, 1H), 7.01 (d, 1H), 4.77-4.73 (m, 1H), 3.90 (s, 3H), 1.33 (d, 6H).

Biological Activity:

IRAK4 Enzymatic DELFIA Assay.

This is an in vitro assay to measure IRAK4 enzymatic activity utilizing the DELFIA (Dissociation-Enhanced Lanthanide Fluorescent Immunoassay, Perkin-Elmer) platform, with the human IRAK4 kinase domain (aa 154-460) construct to characterize IRAK4 inhibitor and control compounds at 0.6 mM ATP (K_{M}). The final amount of enzyme in the assay is 114 pM IRAK4 kinase domain, final concentration of substrate is 200 nM, and final concentration of DMSO is 5%.

The test compound was solubilized in DMSO to a stock concentration of 30 mM. The dose response plates were prepared with a 2 mM primary compound concentration, and then diluted in DMSO in a four-fold series for a total of 10 data points. Compounds were prepared as a 20-fold multiple of the final in-assay concentration

To begin the assay, 45 μL of reaction mixture containing 20 mM HEPES pH=7.5, 5 mM MgCl₂, 0.0025% Brij-35, 600 pM ATP, 228 pM phosphorylated recombinant human IRAK4 kinase domain (aa 154-460; GenBank ID AF445802) were aliquoted into Ultra-Clear Polypropylene, 96-well, U-Bottom Plates (Corning Life Sciences). 5 μL of test compound from the dose-response plate was added to the reaction mixture and incubated for 15 minutes at room temperature. Then 50 μL of 20 mM HEPES pH=7.5, 5 mM MgCl₂, 0.0025% Brij-35, 600 μM ATP, and 400 nM ERM-biotinylated peptide (AGAGRDKYKTLRQIR) were added to start the reaction. The reaction was incubated for 90 minutes at room temperature and stopped by the addition of 25 μL 0.5M EDTA.

100 μL of the reaction mixture was transferred to a streptavidin coated detection plate (EvenCoat Streptavidin Coated Plates, 96-Well, R&D Systems) and incubated for 30 minutes at room temperature. The plates were washed 4 times with 100 μL per well of PBS containing 0.05% Tween-20. Plates were then incubated with 50 μL per well of antibody cocktail of Anti-pERM antibody (Cell Signaling Technology) diluted 1:5000, plus Anti-Rabbit IgG EuN1 at 0.242 μg/ml (Perkin-Elmer Life Sciences) in a solution of 10 mM MOPS pH=7.5, 150 mM NaCl, 0.05% Tween-20, 0.02% NaN₃, 1% BSA, 0.1% Gelatin for 45 minutes. The plates were washed 4× with 100 μL per well of PBS containing 0.05% Tween-20. Then 100 μL per well of DELFIA Enhancement Solution were added to the plate and then read on an EnVision Model 2103 using a 340 nm excitation wavelength and a 665 nm emission detection.

TABLE 1
Biological Activity

| Ex. | IRAK4 |
|---|---|
| No. | IC₅₀ nM |
| 1 | 53 |
| 2 | 66 |
| 3 | 308 |
| 4 | 13 |
| 5 | 35 |
| 6 | 348 |
| 7 | 143 |
| 8 | 168 |
| 9 | 263 |
| 10 | 506 |
| 11 | 560 |
| 12 | 626 |
| 13 | 751 |
| 14 | 856 |
| 15 | 999 |
| 16 | 110 |
| 17 | 111 |
| 18 | 660 |
| 19 | 39 |
| 20 | 150 |
| 21 | 602 |
| 22 | 428 |
| 23 | 151 |
| 24 | 611 |
| 25 | 184 |
| 26 | 950 |
| 27 | 642 |
| 28 | 4 |
| 29 | 148 |
| 30 | 28 |
| 31 | 101 |
| 32 | 35 |
| 33 | 272 |
| 34 | 926 |
| 35 | 266 |
| 36 | 5 |
| 37 | 40 |
| 38 | 162 |
| 39 | 156 |
| 40 | 82 |
| 41 | 883 |
| 42 | 388 |
| 43 | 399 |
| 44 | 234 |
| 45 | 254 |
| 46 | 97 |
| 47 | 755 |
| 48 | 299 |
| 49 | 29 |
| 50 | 514 |
| 51 | 157 |
| 52 | 65 |
| 53 | 43 |
| 54 | 591 |
| 55 | 221 |
| 56 | 288 |
| 57 | 40 |
| 58 | 135 |
| 59 | 919 |
| 60 | 92 |
| 61 | 418 |
| 62 | 739 |
| 63 | 398 |
| 64 | 160 |
| 65 | 419 |
| 66 | 38 |
| 67 | 1390 |
| 68 | 1300 |
| 69 | 36 |
| 70 | 1250 |
| 71 | 1200 |
| 72 | 1100 |
| 73 | 1590 |
| 74 | 2800 |
| 75 | 3080 |
| 76 | 2280 |
| 77 | 1230 |
| 78 | 3040 |
| 79 | 1470 |
| 80 | 1320 |

We claim:

1. A compound of Formula Ia, 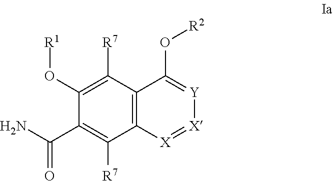
wherein
X and X′ are each independently CR⁶, N or —N⁺—O⁻; Y is independently N, —N⁺—O⁻ or CH; provided that at least one of X, X′ or Y is neither N nor —N⁺—O⁻ and that no more than one of X, X′ or Y is —N⁺—O⁻;
R¹ is C₁-C₆ alkyl or 3- to 7-membered cycloalkyl; wherein said alkyl or cycloalkyl is optionally substituted with one to five halogen, deutero, —OR⁵, —SR⁵, —NR^{11a}R^{11b}, cyano, C₁-C₆alkyl, C₃-C₆cycloalkyl or —C₁-C₆alkoxy;
R² is 3- to 10-membered cycloalkyl; 3- to 10-membered heterocycloalkyl, having one to three heteroatoms; 5- to 10-membered heteroaryl having one to three heteroatoms; or C₆-C₁₂ aryl; wherein said cycloalkyl, heterocycloalkyl, heteroaryl or aryl is optionally substituted with one to five R³ and wherein, if the heteroatom on said heterocycloalkyl or heteroaryl is N, said N is optionally substituted with R⁴;
R³ for each occurrence is independently deuterium, halogen, C₁-C₆alkyl, C₃-C₆cycloalkyl, C₁-C₆alkoxy, oxo, —SR⁵, —NR^{11a}R^{11b}, cyano, or —OR⁵, wherein said alkyl, cycloalkyl or alkoxy is optionally and independently substituted with one to five deuterium, halogen, OR⁵, —SR⁵, —NR^{11a}R^{11b}, cyano, C₁-C₆alkyl, C₃-C₆cycloalkyl or C₁-C₆alkoxy; or two R³ taken together with the respective carbons to which each are bonded form a 3- to 6-membered cycloalkyl or a 4- to 6-membered heterocycloalkyl, wherein said cycloalkyl or heterocycloalkyl is optionally substituted with one to three halogen, deuterium, —OR⁵, —SR⁵, —NR^{11a}R^{11b}, cyano or C₁-C₆alkyl or C₁-C₆alkoxy, wherein the alkyl or alkoxy is optionally substituted with halogen, deuterium, —OR⁵, —SR⁵, —NR^{11a}R^{11b} or cyano; and wherein, if a heteroatom on said heterocycloalkyl is N, said N is optionally substituted with R⁴;
R⁴ is hydrogen, C₁-C₆ alkyl, —C(O)R¹⁰ or —S(O)₂R⁸, wherein the alkyl is optionally substituted with OH, halogen, deuterium, C₁-C₆alkyl, C₁-C₆alkoxy or cyano;
R⁵ is hydrogen or C₁-C₆alkyl, wherein said alkyl is optionally substituted with halogen, deuterium, C₁-C₆alkoxy, C₁-C₆alkylthiolyl, —NR^{11a}R^{11b}, cyano, C₁-C₆alkyl or C₃-C₆cycloalkyl;
each R⁶ is hydrogen, halogen, cyano, —OR⁵, —SR⁵, —NR^{11a}R^{11b}, C₁-C₆alkyl, C₃-C₆cycloalkyl, 3- to 7-membered heterocycloalkyl or 5- to 6-membered heteroaryl or aryl, wherein said alkyl, cycloalkyl, heterocycloalkyl, heteroaryl or aryl is optionally substituted with one to three halogen, —NR^{11a}R^{11b}, —OR⁵, —SR⁵, cyano, C₁-C₃ alkyl, —C(O)R¹⁰ or oxo;
R⁷ is independently hydrogen, methyl, cyano, OCF₃, OMe, CF₃ or halogen;
R⁸ is independently C₁-C₆alkyl, 3- to 6-membered cycloalkyl, 4- to 6-membered heterocycloalkyl, C₆-C₁₀aryl or 5- to 10-membered heteroaryl; wherein said alkyl, cycloalkyl, heterocycloalkyl, aryl or heteroaryl is optionally substituted with one to three deuterium, halogen, OH, C₁-C₆alkoxy, C₁-C₃alkyl (optionally substituted with NR^{11a}R^{11b} or C₁-C₆alkoxy), 3- to 6-membered cycloalkyl, NR^{11a}R^{11b} or cyano;
R¹⁰ is C₁-C₆alkyl, 3- to 6-membered cycloalkyl, 4- to 6-membered heterocycloalkyl, C₆-C₁₀aryl or 5- to 10-membered heteroaryl; wherein said alkyl, cycloalkyl, heterocycloalkyl, aryl or heteroaryl is optionally substituted with one to three deuterium, halogen, OH, C₁-C₆alkoxy, C₁-C₃alkyl (optionally substituted with NR^{11a}R^{11b} or C₁-C₆alkoxy), 3- to 6-membered cycloalkyl, NR^{11a}R^{11b} or cyano; and
R^{11a} and R^{11b} are each independently hydrogen, 3- to 6-membered cycloalkyl or C₁-C₆alkyl, wherein said cycloalkyl or alkyl is optionally substituted with deuterium, C₁-C₆alkoxy or cyano; and if said alkyl is C₂-C₆alkyl, said alkyl is optionally substituted with deuterium, C₁-C₆alkoxy, cyano, halogen or OH;
or a pharmaceutically acceptable salt of said compound or a tautomer of said compound or said salt.

2. The compound of claim 1 wherein X is N, X′ is CR⁶ and Y is CH; X is N, X′ is N and Y is CH; X is N, X′ is CR⁶ and Y is N; X is CR⁶, X′ and Y are N; X and X′ are CR⁶ and Y is N; X is CR⁶ and Y is CH and X′ is N; X and X′ are CR⁶ and Y is CH; or a pharmaceutically acceptable salt of said compound or a tautomer of said salt.

3. A compound of Formula IIa, IIb, IIc, IId, IIe, IIf or IIg, 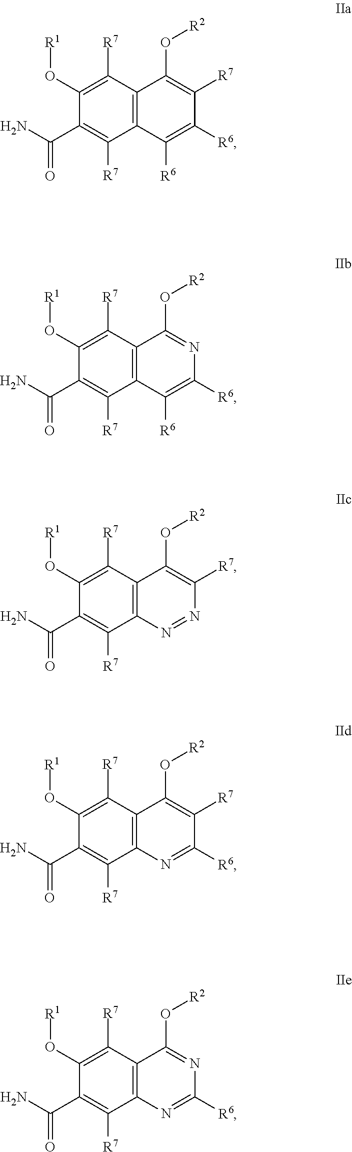 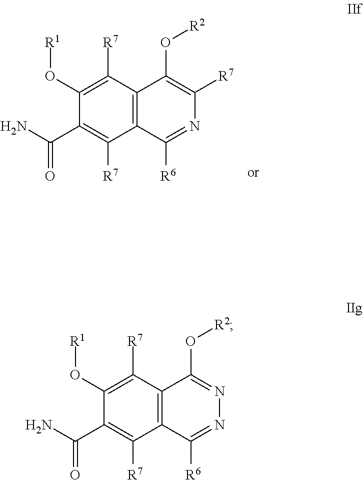
wherein
R¹ is C₁-C₆ alkyl or 3- to 7-membered cycloalkyl; wherein said alkyl or cycloalkyl is optionally substituted with one to five halogen, deutero, —OR⁵ or cyano;
R² is 3- to 7-membered cycloalkyl; 3- to 7-membered heterocycloalkyl, having one to three heteroatoms; 5- to 10 membered heteroaryl having one to three heteroatoms; or C₆-C₁₂ aryl; wherein said cycloalkyl, heterocycloalkyl, heteroaryl or aryl is optionally substituted with one to five R³ and wherein, if a heteroatom on said heterocycloalkyl or heteroaryl is N, said N is optionally substituted with R⁴;
R³ for each occurrence is independently deuterium, halogen, C₁-C₆alkyl, oxo, —OR⁵, wherein said alkyl, is optionally and independently substituted with one to five deuterium, halogen, OR⁵, —SR⁵, —NR^{11a}R^{11b}, cyano, C₁-C₆ alkyl, C₃-C₆ cycloalkyl or C₁-C₆ alkoxy; or two R³ taken together with the respective carbons to which each are bonded form a 3- to 6-membered cycloalkyl or a 4- to 6-membered heterocycloalkyl, wherein said cycloalkyl or heterocycloalkyl is optionally substituted with one to three halogen, deuterium, —OR⁵, —SR⁵, —NR^{11a}R^{11b}, cyano or C₁-C₆alkyl or C₁-C₆alkoxy, wherein the alkyl or alkoxy is optionally substituted with halogen, deuterium, —OR⁵, —SR⁵, —NR^{11a}R^{11b} or cyano; and wherein, if a heteroatom on said heterocycloalkyl is N, said N is optionally substituted with R⁴;
R⁴ is independently hydrogen, C₁-C₆ alkyl, —C(O)R¹⁰ or —S(O)₂R⁸, wherein the alkyl is optionally substituted with OH, halogen, deuterium, C₁-C₆alkyl, C₁-C₆alkoxy or cyano;
R⁵ is independently hydrogen or C₁-C₆alkyl, wherein said alkyl is optionally substituted with halogen, deuterium, C₁-C₆alkoxy, C₁-C₆alkylthiolyl, —NR^{11a}R^{11b}, cyano, C₁-C₆alkyl or C₃-C₆cycloalkyl;
each R⁶ is independently hydrogen, halogen, cyano, —OR⁵, —SR⁵, —NR^{11a}R^{11b}, C₁-C₆alkyl, C₃-C₆cycloalkyl, 3- to 10-membered heterocycloalkyl or 5- to 6-membered heteroaryl or aryl, wherein said alkyl, cycloalkyl, heterocycloalkyl, heteroaryl or aryl is optionally substituted with one to three halogen, —NR^{11a}R^{11b}, —OR⁵, —SR⁵, cyano, C₁-C₃ alkyl, —C(O)R¹⁰ or oxo;
R⁷ is independently hydrogen, methyl, CF₃ or halogen;
R⁸ is C₁-C₃alkyl, 3- to 6-membered cycloalkyl, 4- to 6-membered heteroaryl or C₆-C₁₀aryl, wherein said alkyl, cycloalkyl, heteroaryl or aryl are each optionally substituted with fluoro, 3-6-membered cycloalkyl or C₁-C₃alkyl;
R¹⁰ is C₁-C₆alkyl, 3- to 6-membered cycloalkyl, 4- to 6-membered heterocycloalkyl, C₆-C₁₀aryl or 5- to 6-membered heteroaryl; wherein said alkyl, cycloalkyl, heterocycloalkyl, aryl or heteroaryl is optionally substituted with one to three deuterium, halogen, OH, C₁-C₆alkoxy, C₁-C₃alkyl (optionally substituted with NR^{11a}R^{11b} or C₁-C₆alkoxy), 3- to 6-membered cycloalkyl, NR^{11a}R^{11b} or cyano;
R^{11a} and R^{11b} are each independently hydrogen, 3- to 6-membered cycloalkyl or C₁-C₆alkyl, wherein said cycloalkyl or alkyl is optionally substituted with deuterium, C₁-C₆alkoxy or cyano; and if said alkyl is C₂-C₆alkyl, said alkyl is optionally substituted with deuterium, C₁-C₆alkoxy, cyano, halogen or OH;
or a pharmaceutically acceptable salt of said compound or a tautomer of said compound or said salt.

4. The compound of claims 1, 2 or 3 wherein R² is selected from pyrrolidinyl, pyrrolidin-2-onyl, piperidinyl, piperidin-2-onyl, octahydro-1H-pyrrolo[3,4-c]pyridinyl, oxazolidinyl, oxazolidin-2-onyl, 1,3-oxazinan-2-onyl, imidazolidinyl, imidazolidin-2-onyl, morpholinyl, morpholin-3-onyl, thiazyl, isothiazyl, isothiazolidine-1,1-dioxidyl, 1,2-thiazinane 1,1-dioxidyl, hexahydrocyclopenta[b]pyrrol-2(1H)-onyl, octahydrocyclopenta[c]pyrrolyl, azetidinyl, hexahydro-1H-indol-2(3H)-onyl, octahydro-1H-isoindolyl, azepanyl, tetrahydrofuranyl, 1,3-dioxolanyl, oxetanyl, cyclopropyl, cyclobutyl, cyclopentyl, cyclohexyl, 4-azepanyl, 1,4-oxazepanyl, tetrahydro-2H-pyranyl, 6,7-dihydro-5H-pyrrolo[1,2-a]imidazolyl, cyclohex-2-enyl, or 1,2,3,4-tetrahydroisoquinolinyl; wherein said alkyl, cycloalkyl or heterocycloalkyl is optionally substituted with one to four R³ and wherein, if the heteroatom on said heterocycloalkyl and heteroaryl is N, said N is optionally substituted with R⁴; or a pharmaceutically acceptable salt of said compound or a tautomer of said compound or said salt.

5. The compound of claim 4 wherein R³ for each occurrence is independently fluoro, chloro, C₁-C₃alkyl, oxo or —OR⁵, wherein said alkyl, is optionally and independently substituted with one to five halogen, —OR⁵, C₃-C₆ cycloalkyl or C₁-C₃ alkoxy; or two R³ taken together with the respective carbons to which each are bonded form a 3- to 6-membered cycloalkyl or a 4- to 6-membered heterocycloalkyl, wherein said cycloalkyl or heterocycloalkyl is optionally substituted with one to three halogen, —OR⁵, cyano or C₁-C₆alkyl or C₁-C₆alkoxy, wherein the alkyl or alkoxy is optionally substituted with halogen, —OR⁵; and R⁵ is hydrogen;
or a pharmaceutically acceptable salt of said compound or a tautomer of said compound or said salt.

6. The compound of claim 5 wherein
R⁴ is independently hydrogen, C₁-C₃alkyl, —C(O)R¹⁰ or —S(O)₂R⁸, wherein the alkyl is optionally substituted with OH, halogen, deuterium, C₁-C₆alkyl, C₁-C₆alkoxy or cyano;
R⁸ is C₁-C₃alkyl;
R¹⁰ is C₁-C₃alkyl, 3- to 6-membered cycloalkyl, 4- to 6-membered heterocycloalkyl or 5- to 6-membered heteroaryl; wherein said alkyl, cycloalkyl, heterocycloalkyl or heteroaryl is optionally substituted with one to three deuterium, fluoro, OH, C₁-C₃alkoxy, C₁-C₃alkyl (optionally substituted with NR^{11a}R^{11b} or C₁-C₆alkoxy), 3- to 6-membered cycloalkyl, NR^{11a}R^{11b} or cyano;
or a pharmaceutically acceptable salt of said compound or a tautomer of said compound or said salt.

7. The compound of claim 6 wherein R² is selected from 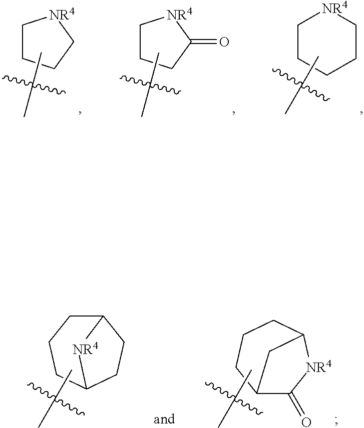
wherein said heterocycloalkyl is optionally substituted with one, two or three R³;
or a pharmaceutically acceptable salt of said compound or a tautomer of said compound or said salt.

8. The compound of claim 7 wherein R³ is independently selected from fluoro, chloro, hydroxyl and methyl; or a pharmaceutically acceptable salt of said compound or a tautomer of said compound or said salt.

9. The compound of claim 8 wherein R¹ is methyl, ethyl, propyl, isopropyl, butyl, t-butyl, cyclopropyl, cyclobutyl, wherein said R¹ is optionally substituted with one, two or three fluoro; or a pharmaceutically acceptable salt of said compound or a tautomer of said compound or said salt.

10. The compound of claim 1 selected from the group consisting of:
4-{[(3R)-1-(cyanoacetyl)piperidin-3-yl]oxy}-6-(propan-2-yloxy)quinoline-7-carboxamide;
4-{[(3R)-1-(cyanoacetyl)piperidin-3-yl]oxy}-6-methoxyquinoline-7-carboxamide;
1-{[(3R,4S)-1-(cyanoacetyl)-4-methylpiperidin-3-yl]oxy}-7-(propan-2-yloxy)isoquinoline-6-carboxamide;
4-{[(3R)-1-(cyanoacetyl)piperidin-3-yl]oxy}-6-ethoxyquinoline-7-carboxamide;
1-{[(1S,5R,8R)-7-oxo-6-azabicyclo[3.2.1]oct-3-en-8-yl]oxy}-7-(propan-2-yloxy)isoquinoline-6-carboxamide;
4-chloro-1-{[(3R)-1-(cyanoacetyl)piperidin-3-yl]oxy}-7-(propan-2-yloxy)isoquinoline-6-carboxamide;
5-(piperidin-4-yloxy)-3-(propan-2-yloxy)naphthalene-2-carboxamide;
4-{[(3R,4S)-1-(cyanoacetyl)-4-methylpiperidin-3-yl]oxy}-6-(propan-2-yloxy)quinoline-7-carboxamide;
4-{[(3R)-1-(cyanoacetyl) pyrrolidin-3-yl]oxy}-6-(propan-2-yloxy)quinoline-7-carboxamide;
4-[(1-acetylpiperidin-3-yl)oxy]-6-(propan-2-yloxy)quinoline-7-carboxamide;
4-({(3R)-1-[(2S)-2-amino-3-cyanopropanoyl]piperidin-3-yl}oxy)-6-(propan-2-yloxy)quinoline-7-carboxamide;
4-{[(3R)-1-(cyanoacetyl)piperidin-3-yl]oxy}-6-(difluoromethoxy)quinoline-7-carboxamide;
5-[(3R)-piperidin-3-yloxy]-3-(propan-2-yloxy)naphthalene-2-carboxamide;
4-{[(3R)-1-(cyanoacetyl)piperidin-3-yl]oxy}-2-methyl-6-(propan-2-yloxy)quinoline-7-carboxamide;
4-{[(3S)-1-(cyanoacetyl)pyrrolidin-3-yl]oxy}-6-(propan-2-yloxy)quinoline-7-carboxamide;
4-cyano-1-{[(3R)-1-(cyanoacetyl)piperidin-3-yl]oxy}-7-(propan-2-yloxy)isoquinoline-6-carboxamide;
4-chloro-1-[(3R)-piperidin-3-yloxy]-7-(propan-2-yloxy)isoquinoline-6-carboxamide;
1-[(1-acetylpiperidin-4-yl)oxy]-7-(propan-2-yloxy)isoquinoline-6-carboxamide;
4-{[1-(methylsulfonyl)piperidin-3-yl]oxy}-6-(propan-2-yloxy)quinoline-7-carboxamide;
4-{[(3R)-1-(cyanoacetyl)piperidin-3-yl]oxy}-6-(cyclobutyloxy)quinoline-7-carboxamide;
4-methyl-1-[(3R)-piperidin-3-yloxy]-7-(propan-2-yloxy)isoquinoline-6-carboxamide;
4-({(3R)-1-[(2R)-2-amino-3-cyanopropanoyl]piperidin-3-yl}oxy)-6-(propan-2-yloxy)quinoline-7-carboxamide;
4-{[(3R)-1-(3-cyanopropanoyl)piperidin-3-yl]oxy}-6-(propan-2-yloxy)quinoline-7-carboxamide;
4-{[(3R)-1-(hydroxyacetyl)piperidin-3-yl]oxy}-2-methyl-6-(propan-2-yloxy)quinoline-7-carboxamide;
4-{[(3S,4S)-3-fluoropiperidin-4-yl]oxy}-6-(propan-2-yloxy)quinoline-7-carboxamide;
1-(piperidin-4-yloxy)-7-(propan-2-yloxy)isoquinoline-6-carboxamide;
4-{[(3R,4S)-3-hydroxypiperidin-4-yl]oxy}-6-(propan-2-yloxy)quinoline-7-carboxamide;
1-{[1-(cyanoacetyl)piperidin-4-yl]oxy}-7-(propan-2-yloxy)isoquinoline-6-carboxamide;
6-(difluoromethoxy)-4-[(3R)-piperidin-3-yloxy]quinoline-7-carboxamide;
1-{[1-(hydroxyacetyl)piperidin-4-yl]oxy}-7-(propan-2-yloxy)isoquinoline-6-carboxamide;
4-bromo-1-[(3R)-piperidin-3-yloxy]-7-(propan-2-yloxy)isoquinoline-6-carboxamide;
4-{[(3R,4S)-4-hydroxypiperidin-3-yl]oxy}-6-(propan-2-yloxy)quinoline-7-carboxamide;
1-{[1-(D-alanyl) piperidin-4-yl]oxy}-7-(propan-2-yloxy)isoquinoline-6-carboxamide
1-{[1-(2-hydroxypropyl)piperidin-4-yl]oxy}-7-(propan-2-yloxy)isoquinoline-6-carboxamide;
1-(piperidin-3-yloxy)-7-(propan-2-yloxy)isoquinoline-6-carboxamide;
4-{[(3R,6S)-6-methylpiperidin-3-yl]oxy}-6-(propan-2-yloxy)quinoline-7-carboxamide;
4-{[(3R)-1-(2-cyanopropanoyl)piperidin-3-yl]oxy}-6-(propan-2-yloxy)quinoline-7-carboxamide;
4-[(2-oxopyrrolidin-3-yl)oxy]-6-(propan-2-yloxy)quinoline-7-carboxamide;
4-(piperidin-4-yloxy)-6-(propan-2-yloxy)quinoline-7-carboxamide;
6-(propan-2-yloxy)-4-[(3S)-pyrrolidin-3-yloxy]quinoline-7-carboxamide;
6-(propan-2-yloxy)-4-(pyrrolidin-3-yloxy)quinoline-7-carboxamide;
4-[(3R)-piperidin-3-yloxy]-6-(propan-2-yloxy)quinoline-7-carboxamide;
6-methoxy-4-[(3R)-piperidin-3-yloxy]quinoline-7-carboxamide;
6-(cyclobutyloxy)-4-[(3R)-piperidin-3-yloxy]quinoline-7-carboxamide;
1-[(3R)-piperidin-3-yloxy]-7-(propan-2-yloxy)isoquinoline-6-carboxamide;
4-{[(3R)-1-(hydroxyacetyl)piperidin-3-yl]oxy}-6-(propan-2-yloxy)quinoline-7-carboxamide;
4-{[(3R)-1-glycylpiperidin-3-yl]oxy}-6-(propan-2-yloxy)quinoline-7-carboxamide;
1-{[(3R)-1-(cyanoacetyl)piperidin-3-yl]oxy}-7-(propan-2-yloxy)isoquinoline-6-carboxamide;
1-{[(3S)-1-(cyanoacetyl)pyrrolidin-3-yl]oxy}-7-(propan-2-yloxy)isoquinoline-6-carboxamide;
1-{[(3R)-1-(cyanoacetyl)pyrrolidin-3-yl]oxy}-7-(propan-2-yloxy)isoquinoline-6-carboxamide;
1-{[(3R,4S)-1-(cyanoacetyl)-3-fluoropiperidin-4-yl]oxy}-7-(propan-2-yloxy)isoquinoline-6-carboxamide;
4-{[(2R,3S)-1-(cyanoacetyl)-2-methylpyrrolidin-3-yl]oxy}-6-(propan-2-yloxy)quinoline-7-carboxamide;
4-{[(3R,4S)-1-(cyanoacetyl)-3-fluoropiperidin-4-yl]oxy}-6-(propan-2-yloxy)quinoline-7-carboxamide;
1-{[(3R)-3-fluoropiperidin-4-yl]oxy}-7-(propan-2-yloxy)isoquinoline-6-carboxamide;
1-[(2-oxopyrrolidin-3-yl)oxy]-7-(propan-2-yloxy) isoquinoline-6-carboxamide;
4-{[(3R)-1-(difluoroacetyl)piperidin-3-yl]oxy}-6-(propan-2-yloxy)quinoline-7-carboxamide;
4-{[(3R)-1-(L-alanyl)piperidin-3-yl]oxy}-6-(propan-2-yloxy)quinoline-7-carboxamide;
4-{[(3R)-1-(methoxyacetyl)piperidin-3-yl]oxy}-6-(propan-2-yloxy)quinoline-7-carboxamide;
6-(propan-2-yloxy)-4-{[(3R)-1-(trifluoroacetyl)piperidin-3-yl]oxy}quinoline-7-carboxamide;
4-{[(3R)-1-propanoylpiperidin-3-yl]oxy}-6-(propan-2-yloxy)quinoline-7-carboxamide;
4-{[(3R)-1-(1,3-oxazol-5-ylcarbonyl)piperidin-3-yl]oxy}-6-(propan-2-yloxy)quinoline-7-carboxamide;
4-{[(3R)-1-(2-methylseryl)piperidin-3-yl]oxy}-6-(propan-2-yloxy)quinoline-7-carboxamide;
4-({(3R)-1-[(2S)-2-hydroxypropanoyl]piperidin-3-yl}oxy)-6-(propan-2-yloxy)quinoline-7-carboxamide;
4-{[(3R)-1-(1H-imidazol-4-ylcarbonyl) piperidin-3-yl]oxy}-6-(propan-2-yloxy)quinoline-7-carboxamide;
4-{[(3R)-1-{[1-(aminomethyl)cyclopropyl]carbonyl}piperidin-3-yl]oxy}-6-(propan-2-yloxy)quinoline-7-carboxamide;
1-{[(3R,4S)-4-methylpiperidin-3-yl]oxy}-7-(propan-2-yloxy)isoquinoline-6-carboxamide;
4-[(1-methylpiperidin-3-yl)oxy]-6-(propan-2-yloxy)quinoline-7-carboxamide;
6-[(2S)-butan-2-yloxy]-4-[(3R)-piperidin-3-yloxy]quinoline-7-carboxamide;
6-(propan-2-yloxy)-4-[(3R)-pyrrolidin-3-yloxy]quinoline-7-carboxamide;
1-[(3-endo)-8-azabicyclo[3.2.1]oct-3-yloxy]-7-(propan-2-yloxy)isoquinoline-6-carboxamide;
7-(propan-2-yloxy)-1-[(3S)-pyrrolidin-3-yloxy]isoquinoline-6-carboxamide;
6-ethoxy-4-[(3R)-piperidin-3-yloxy]quinoline-7-carboxamide;
4-cyano-1-[(3R)-piperidin-3-yloxy]-7-(propan-2-yloxy) isoquinoline-6-carboxamide;
4-{[(3R,4S)-4-methylpiperidin-3-yl]oxy}-6-(propan-2-yloxy)quinoline-7-carboxamide;
1-[(3S)-piperidin-3-yloxy]-7-(propan-2-yloxy)isoquinoline-6-carboxamide;
6-(2-methylpropoxy)-4-[(3R)-piperidin-3-yloxy]quinoline-7-carboxamide;
4-{[(3R,4S)-4-methylpiperidin-3-yl]oxy}-6-(propan-2-yloxy)quinoline-7-carboxamide; and
2-methyl-4-[(3R)-piperidin-3-yloxy]-6-(propan-2-yloxy)quinoline-7-carboxamide;
or pharmaceutically acceptable salts thereof or tautomers of said compounds or salt.

11. A method of treating a mammal, including a human, having a disease or condition selected from the group consisting of autoimmune diseases; inflammatory diseases; autoinflammatory conditions; pain conditions; respiratory; airway and pulmonary conditions; gastrointestinal (GI) disorders; allergic diseases; infection-based diseases; trauma and tissue injury-based conditions; fibrotic diseases; diseases driven by over-activity of IL1 pathways; ophthalmic/ocular diseases; joint, muscle and bone disorders; skin/dermatological diseases; renal diseases; genetic diseases; hematopoietic diseases; liver diseases; oral diseases; metabolic diseases, including diabetes (e.g. Type II) and complications thereof; proliferative diseases; cardiovascular conditions; vascular conditions; neuroinflammatory conditions; neurodegenerative conditions; cancer; sepsis; pulmonary inflammation and injury; or pulmonary hypertension; comprising administering to a mammal in need thereof a therapeutically effective amount of the compound of in any of the preceding claims.

12. The method according to claim 11 wherein the disease or condition is systemic lupus erythematosus (SLE), lupus nephritis, rheumatoid arthritis, psoriasis, atopic dermatitis, gout, cryopyrin-associated periodic syndrome (CAPS), diffuse large B cell lymphoma (DLBCL), chronic kidney disease or acute kidney injury, chronic obstructive pulmonary disorder (COPD), asthma and bronchospasm.

13. A method for treating a disease or condition mediated by or otherwise associated with an IRAK receptor, the method comprising administering to a subject in need thereof a therapeutically effective amount of the compound of any of the preceding claims.

14. A pharmaceutical composition comprising a compound of any of the preceding claims or a pharmaceutically acceptable salt thereof or a tautomer of said compound or said salt and a pharmaceutically acceptable vehicle, diluents or carrier.

15. A pharmaceutical combination comprising a therapeutically effective amount of a composition comprising:
a first compound, the first compound being a compound of any of the preceding claims or a pharmaceutically acceptable salt thereof or a tautomer of said compound or said salt;
a second compound, the second compound being selected from an approved drug or a clinical candidate useful for the treatment of systemic lupus erythematosus (SLE), lupus nephritis, rheumatoid arthritis, psoriasis, atopic dermatitis, gout, cryopyrin-associated periodic syndrome (CAPS), diffuse large B cell lymphoma (DLBCL), chronic kidney disease or acute kidney injury, chronic obstructive pulmonary disorder (COPD), asthma and bronchospasm; and
an optional pharmaceutically acceptable carrier, vehicle or diluent.

16. A pharmaceutical combination comprising a therapeutically effective amount of a composition comprising:
a first compound, the first compound being a compound of any of the preceding claims or a pharmaceutically acceptable salt thereof;
a second compound, the second compound being selected from the group consisting of a non-steroidal anti-inflammatory drugs, immunomodulatory and/or anti-inflammatory agents, antimalarials, antibiotics, Anti-TNFα agents, Anti-CD20 agents, Antidiarrheals, Bile acid binding agents, laxatives, T lymphocyte activation, Anti-IL1 treatments, Glucocorticoid receptor modulators, Aminosalicyic acid derivatives including but not limited to: sulfasalazine and mesalazine, Anti-α4 integrin agents, α1- or α2-adrenergic agonist agents, β-adrenergic agonists, Anticholinergic agents, inhaled long acting beta-agonists, long acting muscarinic antagonists, long acting corticosteroids, leukotriene pathway modulators, H1 receptor antagonists, PDE4 inhibitors, Vitamin D receptor modulators, Nrf2 pathway activators, Modulators of the RAR-related orphan receptor (ROR) family, Modulator and/or antagonists of the chemokine receptors, Prostaglandins, PDE5 inhibitors, Endothelin receptor antagonists, Soluble guanylate cyclase activators, Interferons, Sphingosine 1-phosphate receptor modulators, Inhibitors of the complement pathway, Inhibitors of Janus kinases (one of more of JAK1, JAK2, JAK3, TYK2), Inhibitors of other anti-inflammatory or immunomodulatory kinases, Antioxidants, Inhibitors of IL5, Inhibitors of IL4, Inhibitors of IL13, Anti-lL6 agents, Inhibitors/Antagonists of IL17/IL17R, Antagonists of IL12 and/or IL23, Inhibitors of IL33, Inhibitors of IL9, Inhibitors of GM-CSF, Anti CD4 agents, CRTH2 antagonists, Inhibitors of B lymphocyte stimulator, CD22-specific monoclonal antibodies, Inhibitors of interferon-α, Inhibitor of type I interferon receptors, FcγRIIB agonists, Modified and/or recombinant versions of Heat Shock Protein 10, Inhibitors of the TNF superfamily receptor 12A, Inhibitors of xanthine, Inhibitors of URAT1, agents for the treatment of gout and/or lowering of uric acid levels, Inhibitors of toll-like receptors, Agonists of TLRs, Activators SIRT1, A3 receptor agonists, agents for the treatment of psoriasis, Antifibrotic agents, Prolyl hydroxylase inhibitors, Inhibitors of granulocyte macrophage colony-stimulating factor, Inhibitors of MAdCAM, inhibitors of connective tissue growth factor (CTGF), inhibitors of cathepsin C, inhibitors of soluble epoxide hydrolase, inhibitors of the TNFR1 associated dath domain protein, anti-CD19 agents, anti-B7RP1 agents, inhibitors of ICOS ligand, inhibitors of thymic stromal lymphoprotein, inhibitors of IL2, inhibitors of leucine rich peat neuronal protein 6A, inhibitors of integrins, anti-CD40L agents, modulators of the dopamine D3 receptor, inhibitors/modulators of galectin-3, agents for treating diabetic nephropathy, agents for treating acute kidney injury, modulators of inflammasomes, modulators of bromodomains, modulators of GPR43 or inhibitors of TRP channels and
an optional pharmaceutically acceptable carrier, vehicle or diluent

17. The pharmaceutical combination of claim 16 wherein the second compound is selected from the group consisting of a corticosteroid, hydroxychloroquine, cyclophosphamide, azathioprine, mycophenolate mofetil, methotrexate, janus kinase inhibitor, statin, calcipotriene, angiotensin-converting enzyme inhibitor and angiotensin receptor blocker; and
an optional pharmaceutically acceptable carrier, excipient or diluents.

18. The composition of claim 17 wherein the second compound is a janus kinase inhibitor.

19. The composition of claim 18 wherein the janus kinase inhibitor is selected from ruxolitinib, baricitinib, tofacitinib, Decernotinib, Cerdulatinib, JTE-052, Peficitinib, GLPG-0634, INCB-47986, INCB-039110, PF-04965842, XL-019, ABT-494, R-348, GSK-2586184, AC-410, BMS-911543 and PF-06263276.
